(19) United States
(12) Patent Application Publication
(10) Pub. No.: US 2024/0285672 A1
(43) Pub. Date: Aug. 29, 2024

(54) BETA-GLUCAN FOR PREVENTING AND/OR TREATING FIBROSIS AND RELATED DISEASES

(71) Applicants: GN Corporation Co Ltd, Yamanashi (JP); Sophy Inc., Kochi (JP)

(72) Inventors: TAKASHI ONAKA, Okayama (JP); Samuel JK ABRAHAM, Yamanashi (JP)

(21) Appl. No.: 18/569,553

(22) PCT Filed: Jun. 22, 2022
(86) PCT No.: PCT/JP2022/024984

§ 371 Date: Dec. 12, 2023

(30) Foreign Application Priority Data
Jun. 22, 2021 (JP) 2021-103469

(51) Int. Cl. A61K 31/716; A61K 36/062; A61P 1/16; A61P 11/00; A61P 13/12

(52) U.S. Cl. CPC A61K 31/716; A61K 36/062; A61P 1/16; A61P 11/00; A61P 13/12

(57) ABSTRACT

Compositions and methods for preventing and/or treating fibrosis with a beta-blucan are provided.

### CROSS-REFERENCE TO RELATED APPLICATIONS

This application claims priority to International Patent Application No. PCT/JP2022/024984, filed Jun. 22, 2022, and the benefit of Japanese Application No. 2021-103469, filed Jun. 22, 2021, each of which is incorporated hereby incorporated herein by reference in it's entirety.

### SEQUENCE LISTINGS

The instant application contains a sequence listing which has been submitted in TXT format via EFS-Web, which is hereby incorporated by reference in its entirety. The TXT copy, created on Dec. 12, 2023, is named PCT3163GN_ST25.txt and is 3,817 bytes in size.

### BACKGROUND

### Field of the Invention

The present invention relates to Beta-glucan for preventing and/or treating fibrosis and related disease, to a composition comprising said beta-glucan for preventing and/or treating fibrosis and related disease, and to a method of use of Beta-glucan for preventing and/or treating fibrosis and related disease; and, anti-fibrotic and anti-inflammatory efficacy of Aureobasidium pullulans AFO-202 and N-163-derived Beta 1,3-1,6 biological response modifier glucans in a STAM mouse model of non-alcoholic steatohepatitis.

### Description of the Related Art

Non-alcoholic fatty liver disease (NAFLD) refers to a group of conditions in which there is excess fat accumulation on the liver in people who drink little or no alcohol [A1]. Non-alcoholic steatohepatitis (NASH) is a severe form of NAFLD. NAFLD or NASH progresses to liver fibrosis, liver cirrhosis, liver failure or carcinoma if not treated. Increased prevalence of obesity and metabolic syndrome, diabetes and dysregulated lipid levels all add to the problem of NAFLD and NASH. NAFLD and NASH involve pathologic features such as hepatic steatosis, lobular inflammation, hepatocellular ballooning and liver fibrosis, which ultimately lead to cirrhosis [A1, A2]. There are no definite treatments for NASH. Conventional approaches aim to address the underlying condition such as diabetes and metabolic disease with lifestyle changes, weight reduction, specific medication such as thiazolidinediones, lipid-lowering agents, cytoprotective agents and antioxidants such as vitamin E [A3]. Angiotensin receptor blockers (ARBs) such as telmisartan, which act by modulating transcription factor peroxisome proliferator-activated receptor (PPAR)-γ activity [A4], thereby increasing insulin sensitivity, are increasingly being advocated. However, the underlying aetiology and disease pathogenesis need more holistic approaches.

Beta glucans are potent biological response modifiers that have been proven to be effective in modulating dysregulated metabolism by regulating blood glucose and lipid levels. An Aureobasidium pullulans (black yeast) AFO-202 strain-derived 1,3-1,6 beta glucan has been demonstrated to decrease HbA1c to normal values and decrease fasting and post-prandial blood in human clinical studies [A5, A6]. This GMP-manufactured beta glucan has been proven to regulate lipid levels of triglycerides, total cholesterol and HDL cholesterol in another human clinical study [A7]. Another variant of the 1,3-1,6 beta glucan has been derived from a novel strain of Aureobasidium pullulans N-163, which in vitro studies has shown to have a positive effect on lipid metabolism (data unpublished).

### CITATION LIST

### Patent Literature

[PTL-1] KR100707917B1

### Non Patent Literature

[NPL-1] Brockman D A, Chen X, Gallaher D D. Consumption of a high β-glucan barley flour improves glucose control and fatty liver and increases muscle acylcarnitines in the Zucker diabetic fatty rat. Eur J Nutr. 2013 October; 52(7): 1743-53.

### SUMMARY OF THE INVENTION

The present invention relates to the following:
1. A composition for preventing and/or treating fibrosis, comprising a beta-glucan.
2. The composition of item 1, in which the beta-glucan comprises a beta-glucan produced by Aureobasidium pullulans N-163 (NITE BP-03377).
3. The composition of item 2, in which the beta-glucan further comprises a beta-glucan produced by Aureobasidium pullulans AFO-202 (FERM BP-19327).
4. The composition of item 1, in which the beta-glucan consists of a beta-glucan produced by Aureobasidium pullulans N-163 (NITE BP-03377) and a beta-glucan produced by Aureobasidium pullulans AFO-202 (FERM BP-19327).
5. The composition of any one of items 1 to 4, which is used to prevent and/or treat non-alcoholic steato hepatitis (NASH).
6. A composition for improving gut microbiota, comprising a beta-glucan produced by Aureobasidium pullulans N-163 (NITE BP-03377).
7. The composition according to item 6, further comprising a beta-glucan produced by Aureobasidium pullulans AFO-202 (FERM BP-19327).
8. The composition according to item 6 or 7, wherein the improvement of gut microbiota comprises a decrease of Akkermansia with an increase of beneficial bacteria including Lactobacillus in a gut.
9. The composition according to any one of items 6 to 8, wherein the composition is for prophylactic, ameliorating and/or curative treatment of cancers, and/or fibrosis.
10. A composition for balancing amino acids to beneficial levels, comprising a beta-glucan produced by Aureobasidium pullulans N-163 (NITE BP-03377).
11. The composition according to item 10, further comprising a beta-glucan produced by Aureobasidium pullulans AFO-202 (FERM BP-19327).
12. The composition according to item 10, wherein the composition increases Tryptophan and/or decreases Isoleucine, Leucine, and/or Spermidine.
13. The composition according to item 11, wherein the composition increases Ornithine and/or decreases Tryptophan and/or Phenylalanine.

### Effects of the Invention

In the present study, we report the anti-fibrotic and anti-inflammatory effects of N-163-derived beta glucan in a Stelic Animal Model (STAM), which recapitulates the disease pathology of NASH that occurs in humans.

Based on our earlier reports of clinical and pre-clinical studies in which AFO-202 and N-163 produced biological response modifier glucans either individually or in combination yield a beneficial outcome, we herein report the gut microbiota and fecal metabolome in a Stelic Animal Model (STAM) model which is one of the most metabolically stressed animal models.

### BRIEF DESCRIPTION OF THE DRAWINGS

FIG. 1. Decrease in non-esterified fatty acids (NEFA) greater in N-163 beta glucan group compared to control.

FIG. 2. Decrease in IL-8 greater in N-163 beta glucan group compared to control. IL8 levels decreased after N-163 consumption compared to Control. The results show that SD rats fed with beta glucan N-163 for 28 days show significant decrease in IL8 levels when compared with control group.

FIG. 3. Decrease in IL-6 from immature iPS derived dendritic cells greater in N-163 beta glucan group compared to control.

FIG. 4. Decrease in IL-6 from mature iPS derived dendritic cells greater in N-163 beta glucan group compared to control.

FIG. 5. Decrease in IL-8 from immature iPS derived dendritic cells greater in N-163 beta glucan group compared to control.

FIG. 6. Decrease in non-esterified fatty acids (NEFA) greater in N-163 beta glucan group compared to control and AFO-202 beta glucan. NEFA levels decreased after N-163 consumption compared to AFO-202. The results show that diabetic model KK-Ay mice fed with beta glucan N-163 for 28 days show significant decrease in NEFA levels when compared with control group and AFO-202 group. NEFA is also called as Free Fatty Acid.

FIG. 7. Illustration of study plan of clinical study of AFO-202 and combination of AFO-202 and N-163.

FIG. 8A. Decrease in Ferritin more in AFO-202+N-163 (Gr. II) compared to AFO-202 (Gr.I). A Subgroup: Subjects who consumed Nichi Glucan AFO-202 and N-163 for 21 days and stopped.

FIG. 8B. Decrease in Ferritin more in AFO-202+N-163 (Gr. II) compared to AFO-202 (Gr.I). A Subgroup: Subjects who consumed Nichi Glucan AFO-202 and N-163 for 21 days and stopped.

FIG. 8C. Decrease in Ferritin more in AFO-202+N-163 (Gr. II) compared to AFO-202 (Gr.I). B Subgroup: Subjects who consumed Nichi Glucan AFO-202 and N-163 for 35 days.

FIG. 9A. Total cholesterol decrease is significantly advantageous in AFO-202+N-163 (Gr. II) over AFO-202 (Gr. I).

FIG. 9B. Total cholesterol decrease is significantly advantageous in AFO-202+N-163 (Gr. II) over AFO-202 (Gr. I). A Subgroup: Subjects who consumed Nichi Glucan AFO-202 and N-163 for 21 days and stopped.

FIG. 9C. Total cholesterol decrease is significantly advantageous in AFO-202+N-163 (Gr. II) over AFO-202 (Gr. I). A Subgroup: Subjects who consumed Nichi Glucan AFO-202 and N-163 for 21 days and stopped.

FIG. 9D. Total cholesterol decrease is significantly advantageous in AFO-202+N-163 (Gr. II) over AFO-202 (Gr. I). B Subgroup: Subjects who consumed Nichi Glucan AFO-202 and N-163 for 35 days.

FIG. 9E. Total cholesterol decrease is significantly advantageous in AFO-202+N-163 (Gr. II) over AFO-202 (Gr. I). B Subgroup: Subjects who consumed Nichi Glucan AFO-202 and N-163 for 35 days.

FIG. 10A. LDL cholesterol decrease is significantly advantageous in AFO-202+N-163 (Gr. II) over AFO-202 (Gr. I).

FIG. 10B. LDL cholesterol decrease is significantly advantageous in AFO-202+N-163 (Gr. II) over AFO-202 (Gr. I). A Subgroup: Subjects who consumed Nichi Glucan AFO-202 and N-163 for 21 days and stopped.

FIG. 10C. LDL cholesterol decrease is significantly advantageous in AFO-202+N-163 (Gr. II) over AFO-202 (Gr. I). A Subgroup: Subjects who consumed Nichi Glucan AFO-202 and N-163 for 21 days and stopped.

FIG. 10D. LDL cholesterol decrease is significantly advantageous in AFO-202+N-163 (Gr. II) over AFO-202 (Gr. I). B Subgroup: Subjects who consumed Nichi Glucan AFO-202 and N-163 for 35 days.

FIG. 10E. LDL cholesterol decrease is significantly advantageous in AFO-202+N-163 (Gr. II) over AFO-202 (Gr. I). B Subgroup: Subjects who consumed Nichi Glucan AFO-202 and N-163 for 35 days.

FIG. 11A. Galectin-3 decrease is significantly greater in AFO-202+N-163 (Gr. II) over AFO-202 (Gr. I). A Subgroup: Subjects who consumed Nichi Glucan AFO-202 and N-163 for 21 days and stopped.

FIG. 11B. Galectin-3 decrease is significantly greater in AFO-202+N-163 (Gr. II) over AFO-202 (Gr. I). A Subgroup: Subjects who consumed Nichi Glucan AFO-202 and N-163 for 21 days and stopped.

FIG. 11C. Galectin-3 decrease is significantly greater in AFO-202+N-163 (Gr. II) over AFO-202 (Gr. I). B Subgroup: Subjects who consumed Nichi Glucan AFO-202 and N-163 for 35 days.

FIG. 12A. HbA1c decrease is significantly greater in AFO-202+N-163 (Gr. II) over AFO-202 (Gr. I) in A subgroup. A Subgroup: Subjects who consumed Nichi Glucan AFO-202 and N-163 for 21 days and stopped. B subgroup: Subjects who consumed Nichi Glucan AFO-202 and N-163 for 35 days.

FIG. 12B. HbA1c decrease is significantly greater in AFO-202+N-163 (Gr. II) over AFO-202 (Gr. I) in A subgroup. A Subgroup: Subjects who consumed Nichi Glucan AFO-202 and N-163 for 21 days and stopped. B subgroup: Subjects who consumed Nichi Glucan AFO-202 and N-163 for 35 days.

FIG. 13A. Glycated Albumin (GA) decrease is significantly greater in AFO-202+N-163 (Gr. II) over AFO-202 (Gr. I). A Subgroup: Subjects who consumed Nichi Glucan AFO-202 and N-163 for 21 days and stopped. B subgroup: Subjects who consumed Nichi Glucan AFO-202 and N-163 for 35 days.

FIG. 13B. Glycated Albumin (GA) decrease is significantly greater in AFO-202+N-163 (Gr. II) over AFO-202 (Gr. I). A Subgroup: Subjects who consumed Nichi Glucan AFO-202 and N-163 for 21 days and stopped. B subgroup: Subjects who consumed Nichi Glucan AFO-202 and N-163 for 35 days.

FIG. 14A. Body weight and liver weight showing no significant difference between the groups.

FIG. 14B. Body weight and liver weight showing no significant difference between the groups.

FIG. 15. Plasma ALT (mg/dL) levels greatly decreased in the telmisartan and N-163 groups.

FIG. 16A. Sirius red staining showing significantly decreased positive staining area in the AFO-202+N-163 and N-163 groups compared with all the other groups.

FIG. 16B. Representative photomicrographs of F4/80-immunostained liver sections; Upper panel: Magnification: ×200; Lower panel: Magnification: ×400. F4/80 immunostaining of liver sections from the vehicle group demonstrated accumulation of F4/80+ cells (macrophages associated with inflammation) in the liver lobule.

FIG. 17A. Average positive stained area for fibrosis showing significantly decreased positive staining area in the AFO-202+N-163 and N-163 groups compared with all the other groups. Original magnification for quantification: ×200. Mean+SD. Bonferroni Multiple Comparison Test.

FIG. 17B. F4/80 immunostaining score for inflammatory macrophages was least in the N-163 group compared to the other groups.

FIG. 18. Representative photomicrographs of HE-stained liver sections.

FIG. 19A. NAFLD, steatosis, inflammation and ballooning scores in the various groups based on H and E staining.

FIG. 19B. NAFLD, steatosis, inflammation and ballooning scores in the various groups based on H and E staining.

FIG. 19C. NAFLD, steatosis, inflammation and ballooning scores in the various groups based on H and E staining.

FIG. 19D. NAFLD, steatosis, inflammation and ballooning scores in the various groups based on H and E staining.

FIG. 20. Graphical abstract of illustration of Hepatoprotective effects of Aureobasidium pullulans derived beta glucans in non-alcoholic steatohepatitis.

FIG. 21. Study groups and fecal samples analysis reference numbers given for gut microbiome and metabolome analysis.

FIG. 22A. Alpha-Diversity Indices. A. Simpson index; B. Shannon's index reflecting the diversity of zOTUs in samples. Both the indices indicate that Gr. 4 (AFO-202+N-163) had the highest bacterial abundance post-intervention.

FIG. 22B. Alpha-Diversity Indices. A. Simpson index; B. Shannon's index reflecting the diversity of zOTUs in samples. Both the indices indicate that Gr. 4 (AFO-202+N-163) had the highest bacterial abundance post-intervention.

FIG. 23A. Top-half of index of the most abundant taxa across species levels, overlapping bottom-half in FIG. 23B.

FIG. 23B. Bottom-half of index of the most abundant taxa across species levels, overlapping top-half in FIG. 23A.

FIG. 24A. Differences pre and post-intervention in the read count of selected bacteria between A. Enterobacteria; B. Firmicutes; C. Turicibacter; D. Bilophila. E. Lactobacillus; F. Proteobacteria and G. Akkermansia.

FIG. 24B. Differences pre and post-intervention in the read count of selected bacteria between A. Enterobacteria; B. Firmicutes; C. Turicibacter; D. Bilophila. E. Lactobacillus; F. Proteobacteria and G. Akkermansia.

FIG. 24C. Differences pre and post-intervention in the read count of selected bacteria between A. Enterobacteria; B. Firmicutes; C. Turicibacter; D. Bilophila. E. Lactobacillus; F. Proteobacteria and G. Akkermansia.

FIG. 24D. Differences pre and post-intervention in the read count of selected bacteria between A. Enterobacteria; B. Firmicutes; C. Turicibacter; D. Bilophila. E. Lactobacillus; F. Proteobacteria and G. Akkermansia.

FIG. 24E. Differences pre and post-intervention in the read count of selected bacteria between A. Enterobacteria; B. Firmicutes; C. Turicibacter; D. Bilophila. E. Lactobacillus; F. Proteobacteria and G. Akkermansia.

FIG. 25A. Differential abundance analysis, log 2 fold change results for each group before and after intervention. A. Control; B. N-163; C. AFO-202+N-163 and D. Telmisartan.

FIG. 25B. Differential abundance analysis, log 2 fold change results for each group before and after intervention. A. Control; B. N-163; C. AFO-202+N-163 and D. Telmisartan.

FIG. 25C. Differential abundance analysis, log 2 fold change results for each group before and after intervention. A. Control; B. N-163; C. AFO-202+N-163 and D. Telmisartan.

FIG. 25D. Differential abundance analysis, log 2 fold change results for each group before and after intervention. A. Control; B. N-163; C. AFO-202+N-163 and D. Telmisartan.

FIGS. 26A1 and 26A2. Score plots of principal component analysis (PCA) and compounds with a VIP value of 1 or higher in the OPLS-DA of the different groups A. Control; B. N-163; C. AFO-202+N-163 and D. Telmisartan.

FIGS. 26B1 and 26B2 Score plots of principal component analysis (PCA) and compounds with a VIP value of 1 or higher in the OPLS-DA of the different groups A. Control; B. N-163; C. AFO-202+N-163 and D. Telmisartan.

FIGS. 26C1 and 26C2. Score plots of principal component analysis (PCA) and compounds with a VIP value of 1 or higher in the OPLS-DA of the different groups A. Control; B. N-163; C. AFO-202+N-163 and D. Telmisartan.

FIGS. 26D1 and 26D2. Score plots of principal component analysis (PCA) and compounds with a VIP value of 1 or higher in the OPLS-DA of the different groups A. Control; B. N-163; C. AFO-202+N-163 and D. Telmisartan.

FIG. 27A. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27B. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27C. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27D. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27E. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27F. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27G. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIG. 27H. Peak height of the detected compounds after normalization. A. Succinic acid, B. Phosphoric acid, C. Fructose; D. Tryptophan; E. Isoleucine; F. Leucine, G. Phenylalanine, H. Methionine, I. Spermidine and J. Ornithine. TMS indicates trimethylsilylation, meto indicates methoxymated derivatization (**significant; *not significant; p-value significance <0.05).

FIGS. 28A1 and 28A2. Top-half and bottom-half of, overlapping, Euclidean distance hierarchical clustering analysis demonstrating the different intensity levels of characteristic metabolites in. N-163; AFO-202+N-163; Comparison images of Telmisartan and Control available as Figure. 31.

FIGS. 28B1 and 28B2. Top-half and bottom-half of, overlapping, Euclidean distance hierarchical clustering analysis demonstrating the different intensity levels of characteristic metabolites in. N-163; AFO-202+N-163; Comparison images of Telmisartan and Control available as Figure. 31.

FIG. 29A. Most abundant taxa at the phyla (A), genus (B), and species (C) levels in the different groups of the study.

FIG. 29B. Most abundant taxa at the phyla (A), genus (B), and species (C) levels in the different groups of the study.

FIG. 29C. Most abundant taxa at the phyla (A), genus (B), and species (C) levels in the different groups of the study.

FIG. 30A. Principal component analysis (PCA) of: A, B. all the ten samples (five groups-pre and post-intervention)-A. Score plot; B. Loading plot; Study groups: Control/Vehicle: 4-Baseline, 14-Post intervention; N-163: 6-Baseline; 16-Post intervention; AFO-202+N-163: 8-Baseline F18S-18-Post intervention; Telmisartan: 8-Baseline, 18-Post intervention.

FIG. 30B. Principal component analysis (PCA) of: A, B. all the ten samples (five groups-pre and post-intervention)-A. Score plot; B. Loading plot; Study groups: Control/Vehicle: 4-Baseline, 14-Post intervenetion; N-163: 6-Baseline; 16−Post intervention; AFO-202+N-163: 8-Baseline F18S-18-Post intervention; Telmisartan: 8-Baseline, 18-Post intervention.

FIGS. 31A1 and 31A2. Top-half and bottom-half of, overlapping, Euclidean distance hierarchical clustering analysis demonstrating the different intensity levels of characteristic metabolites in A. Control; B. Telmisartan.

FIGS. 31B1 and 31B2. Top-half and bottom-half of, overlapping, Euclidean distance hierarchical clustering analysis demonstrating the different intensity levels of characteristic metabolites in A. Control; B. Telmisartan.

FIG. 32. Decrease in Free fatty Acids (FFA) greater in N-163 beta glucan group.

FIG. 33. Greater decrease in TNF-Alpha in N-163+AFO-202 beta glucan group than N-163 group.

FIG. 34. Decrease in MCP-1 in N-163+AFO-202 beta glucan group than N-163 group.

FIG. 35. Decrease in Alpha-SMA in N-163 group.

FIG. 36. Decrease in TIMP-1 in N-163 group.

FIG. 37. Greater Decrease in IL-6 in N-163+AFO-202 beta glucan group than N-163 group.

FIG. 38. Greater Decrease in MIP-2 in N-163+AFO-202 beta glucan group than N-163 group.

FIG. 39. The morphology of the normal human dendritic cells (NHDC: CC-2701) expressing HLA-DR, CD11C, CD86, CD80, and CD14 treated with five types of beta-glucans.

FIG. 40A. mRNA expression of TNF-α and IL-6 on the normal human dendritic cells (NHDC: CC-2701) expressing HLA-DR, CD11C, CD86, CD80 and CD14 treated with five types of beta-glucans.

FIG. 40B. mRNA expression of TNF-α and IL-6 on the normal human dendritic cells (NHDC: CC-2701) expressing HLA-DR, CD11C, CD86, CD80 and CD14 treated with five types of beta-glucans.

FIG. 41. Decrease in IL-4 significant in N-163 beta glucan group compared to other beta glucans.

FIG. 42. Decrease in IL-10 greater in N-163 beta glucan group compared to other beta glucans.

FIG. 43. Decrease in IL-13 greater in N-163 beta glucan group compared to other beta glucans.

FIG. 44. Based on the metabolome data of AFO-202, N-163 and combination of AFO-202 and N-163 a proprietary mathematical information technology was used to analyze the metabolome data, which was then converted into the enzyme abundance before and after the metabolite formation, and the imprinting and significant pathways were estimated.

### DETAILED DESCRIPTION OF INVENTION

The glucan used in the present invention can be a glucan derived from Aureobasidium pullulans strain APNN-M163 (Also referred to herein as “strain M163”, or “strain N-163”), and preferably β-1,3-1,6 glucan derived from N-163 (Also referred to herein simply as “N-163 glucan” or “N-163 beta glucan”). “Aureobasidium pullulans strain APNN-M163” has been deposited at the Patent Microorganisms Depositary Center, National Institute of Technology and Evaluation (Room. 122, 2-5-8, Kazusa Kamatari, Kisarazu City, Chiba, 292-0818 Japan), under the deposit number NITE P-03377, on Feb. 9, 2021.

While the domestic deposition was made on Feb. 9, 2021, Aureobasidium pullulans strain APNN-M163 has then been transferred to international deposition at the International Patent Organism Depositary, National Institute of Technology and Evaluation (Room. 122, 2-5-8, Kazusa Kamatari, Kisarazu-shi, Chiba, 292-0818 Japan) on Sep. 14, 2021 with the accession number: NITE BP-03377.

The glucan produced by N-163 strain was estimated to have the following chemical structure (Japanese Patent Application No. 2021-187255).

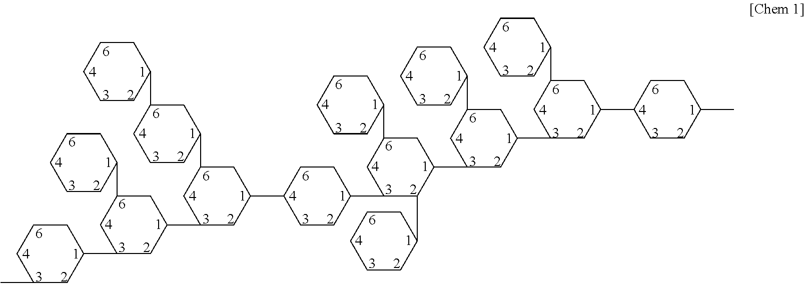

The glucan used in the present invention can also be a glucan derived from Aureobasidium pullulans strain FO-68 (Also referred to herein as “strain AFO 202”), and preferably β-1,3-1,6 glucan derived from FO-68 (Also referred to herein simply as “AFO-202 glucan” or “AF 202 beta glucan”). “Aureobasidium pullulans strain FO-68” has been deposited at the Patent Biological Depository Center, National Institute of Advanced Industrial Science and Technology, under the deposit number FERMP-19327.

While the domestic deposition was made on Apr. 23, 2003, Aureobasidium pullulans strain FO-68 has then been transferred to international deposition at the International Patent Organism Depositary, National Institute of Technology and Evaluation (Room. 120, 2-5-8, Kazusa Kamatari, Kisarazu-shi, Chiba, 292-0818 Japan) on Apr. 21, 2021 with the accession number: FERM BP-19327.

Aureobasidium pullulans strain FO-68 is also called as Aureobasidium strain FERM P-18099.

The glucan produced by AFO-202 strain was estimated to have the following chemical structure (Japanese Patent Application No. 2021-187255).

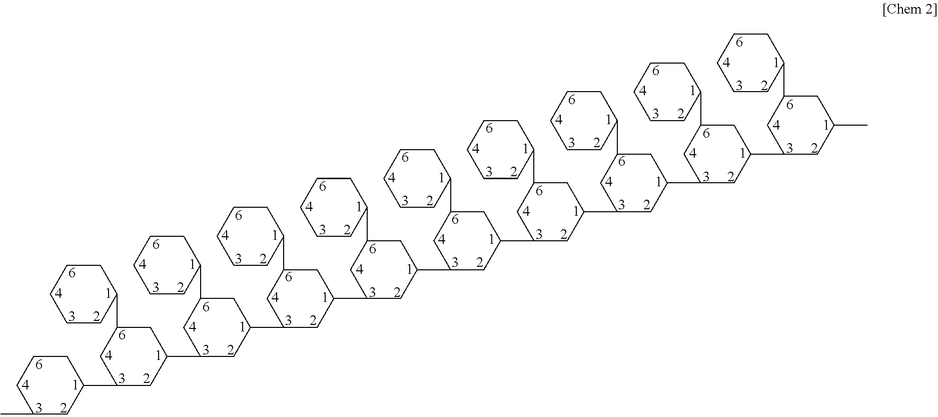

N-163 is a potent agent of resolving diseases by modulating an overacting inflammatory system. This anti inflammation prevents (i) abnormal infiltration macrophages, (ii) accumulation of fibroblasts and that of (iii) extra cellular matrix. By these three effects paves way for resolving fibrosis eventually fibrosis is what that leads to organ failure and chronic inflammation leads to cancer in fibrotic organs (Jun et al, JCI 2018, Resolution of organ fibrosis).

AFO-202 being a regulator of metabolism, by preventing excess of fat or sugar or its derivatives in blood, which leads to metabolites and fat accumulation in organs such as liver This, majorly plays a preventive or prophylactic role and when combined with N-163 which is more anti-inflammatory, becomes a win-win combi for both prevention and treatment.

The composition of the present invention exerts its function when ingested by mammals including humans. The term “ingestion” as used herein is not limited to any administration route as long as it can enter the human body, and is realized by all known administration methods such as oral administration, tube administration, and enteral administration. Typically, oral ingestion and enteral ingestion via the digestive tract are preferable.

The dose of the present invention can be appropriately set in consideration of various factors such as administration route, age, body weight, and symptoms. The dose of the composition of the present invention is not particularly limited, but the amount of glucan is preferably 0.05 mg/kg/day or more, more preferably 0.5 mg/kg/day or more, particularly preferably 1.0 mg/kg/day. However, when ingested over a long period of time, the amount may be smaller than the preferable amount described above. In addition, the glucan used in the present invention has a sufficient dietary experience, and there is no problem in terms of safety. Therefore, an amount far exceeding the above amount (for example, 10 mg/kg/day or more).

The composition of the present invention can be used as a food or drink. The composition of the present invention, as a special-purpose food such as a food for specified health use and a nutritionally functional food, by administering to animals such as humans, treatment or prevention can be achieved against various diseases related to fibrosis.

When the composition of the present invention is used as food or drink, the type of food or drink is not particularly limited. Further, the shape of the food or drink is not particularly limited, and may be any shape of food or drink that is usually used. For example, it may be in any form such as solid form (including powder and granule form), paste form, liquid form and suspension form, and is not limited to these forms.

When used as a pharmaceutical, a dosage form that can be orally administered is preferable because the composition of the present invention reaches the intestine. Examples of preferable dosage forms of the drug according to the present invention include tablets, coated tablets, capsules, granules, powders, solutions, syrups, troches and the like. These various preparations are prepared according to a conventional method by using glucan, which is the active ingredient, an excipient, a binder, a disintegrating agent, a lubricant, a coloring agent, a flavoring agent, a solubilizing agent, a suspending agent, a coating agent, etc. It can be formulated by admixing the auxiliaries usually used in the technical field of pharmaceutical formulation.

In some embodiment, the present invention can be used in combination with other food, drink, drugs and any other substances in order to enhance the efficacy of the present invention.

### Examples

Hereinafter, the present invention will be described more specifically based on the following literature studies and examples. It should be noted that this embodiment does not limit the present invention.

Animal models are most useful to investigate the etiopathogenesis. For NASH, animal models are developed by genetic engineering models, inducing genetic leptin-mutation, dietary methionine choline deficiency (MCD) or a long-term high fat diet (HFD). However, the clinicopathogenesis that occurs in the progression of fatty liver, NASH, fibrosis to HCC under a diabetic background which occurs in humans, is not recapitulated in these models. Stelic Animal Model (STAM™) model is an animal model that recapitulates the disease progression of that which occurs in human NASH/HCC. In this model, C57BL/6 mice aged two days are given a single dose of streptozotocin to reduce the insulin secretory capacity. When the mice turn four weeks of age they are started on a high-fat diet feeding. This model has a background of late type 2 diabetes which progresses into fatty liver, NASH, fibrosis and consequently HCC. In this study we have employed the STAM™ model to study the anti-fibrotic and anti-inflammatory effects of beta glucans from a black yeast, Aureobasidium pullulans.

### REFERENCES

1. Fujii M, et al., Fuji M_Med Mol Morph_2013, DOI 10.1007/s00795-013-0016-1.
2. STAM Model, https://www.smccro-lab.com/jp/service/service_disease_area/stam.html.
3. Middleton et al., Nat Sci Rep (2018)8:17257 DOI: 10.1038/s41598-018-35653-4.

### Example 1: F1S Study (Part I)

F1S study data (Part I) show significance of N-163 in fibrosis & inflammation linked to Liver & Kidney diseases.

### Materials and Methods

Beta Glucan N-163 administered to Kk-Ay mice and compared with control (administration of water for injection).
The Rats were sacrificed after 28 days.
NEFA levels were analyzed.

### Results:

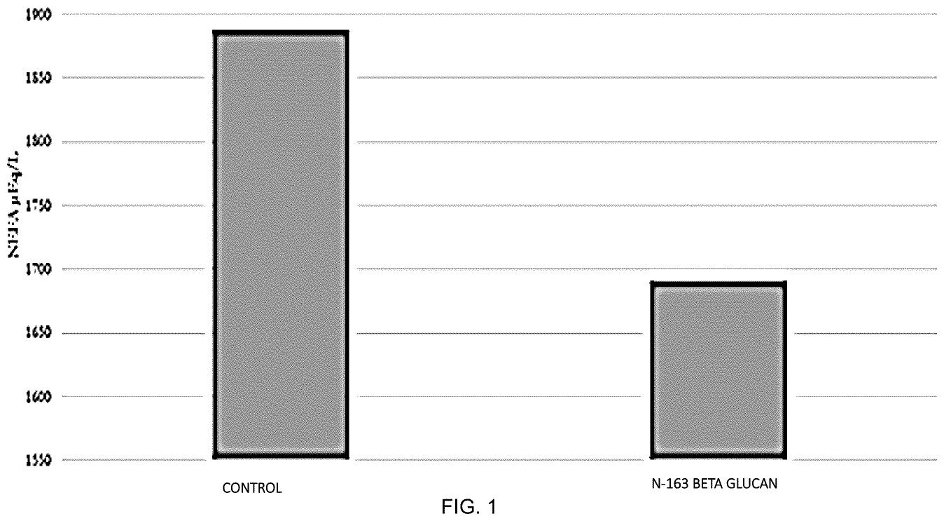

See FIG. 1.

The mean value of NEFA in Kk-Ay mice after administration orally for 28 days was:
Control (Water for injection): 1,888 uEq/L
N-163 Beta Glucan: 1,691 uEq/L.

### Discussion

Decrease in NEFA is related to improvement in insulin sensitivity, according to Daniele, G. et al., Diabetes 2014 August; 63(8): 2812-2820.
NEFA levels decrease is related to NASH significance, according to Zhang, J W. et al., Scientific Reports volume 4, Article number: 5832 (2014), and Paschos P. et al., Hippokratia. 2009 January March; 13(1): 9 19.
Decrease in NEFA is related to kidney disease, according to Gai Z. B. et al., Nutrients. 2019 April; 11(4): 722.

High NEFA concentrations induce a vascular proinflammatory phenotype including the effect of 9 and 13 hydroxyoctadecadienoic acids and other lipid mediators (https://pubmed ncbi nlm nih gov/22916905 High NEFA also predisposes to metabolic diseases and fatty liver (https://www ncbi nlm nih gov/pmc/articles/PMC 2874689 and https://pubmed ncbi nlm nih gov/10952470.

Reduction in Non-esterified fatty acids (NEFA) improves whole body's metabolism by acting on mitochondrial function, improves insulin sensitivity and thus combats inflammation and development of metabolic diseases. Also, increased level of fatty acids has been associated with chronic kidney disease and fibrosis. So, reduction of NEFA is beneficial in preventing and/or resolving fatty liver disease and chronic kidney disease.

### Example 2: F2S Study (Part I)

F2S study data (Part I) show advantages of N 163 in Liver and Lung fibrosis.

### Materials and Methods

Nichi Glucan N 163 was administered to 6 SD Male Rats and compared with control (administration of water for injection).
The Rats were sacrificed after 15 days.
IL 8 levels analyzed in blood.

### Results:

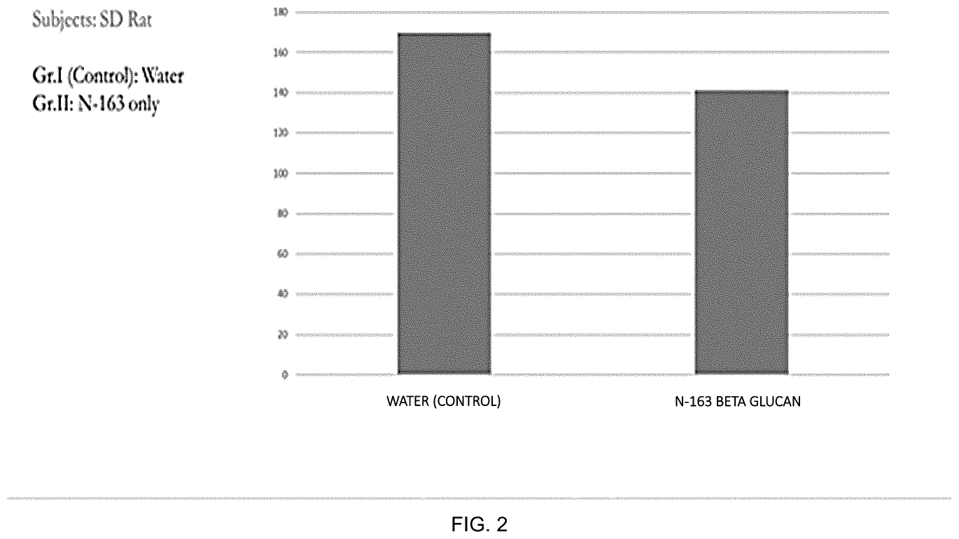

See FIG. 2.

The mean value of IL-8 in SD rats after administration orally for 28 days was:
Control (Water for injection): 170 pg/dL
N-163 Beta Glucan: 141 pg/dL.

### Discussion

Activation of IL 8 is linked to progression of liver fibrosis in patients, according to PLOS One. 2011; 6(6): e21381.
Decrease in IL8 levels is related to pulmonary fibrosis, according to Am J Physiol Lung Cell Mol Physiol. 2018 Jan. 1; 314(1): L127 L136.

Since Interleukin-8 (IL-8) is a chemoattractant cytokine, especially known as the key mediator associated with inflammation, decrease in IL-8 makes N-162 a potential anti-inflammatory agent. Activation of IL-8 is linked to progression of liver fibrosis and idiopathic pulmonary fibrosis. Hence decrease in IL-8 makes N-163 beta glucan a potential anti-fibrosis agent in liver and lung fibrosis.

### Example 3: F11S Study (Part I)

F11S study data (Part I) show advantages of N-163 in liver fibrosis, lung fibrosis, kidney fibrosis, with potentials in Rheumatoid arthritis and chronic aging related morbidities.

### Materials and Methods:

MiCAN Technologies Inc., Japan has developed an induced pluripotent stem (iPS) cell line derived myeloid lineage-dendritic cells which were cultured. N-163 Beta Glucan was added to stimulate the cell lines. IL-6 and IL-8 levels secreted in the supernatant was measured by ELISA.

### Results:

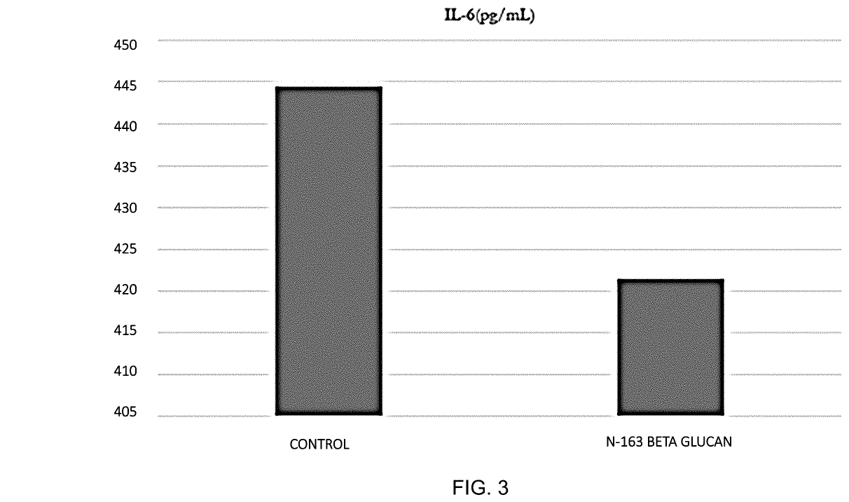

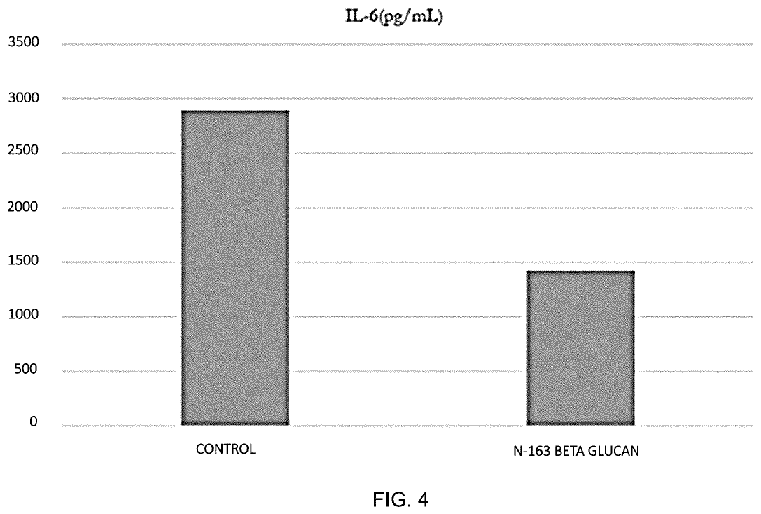

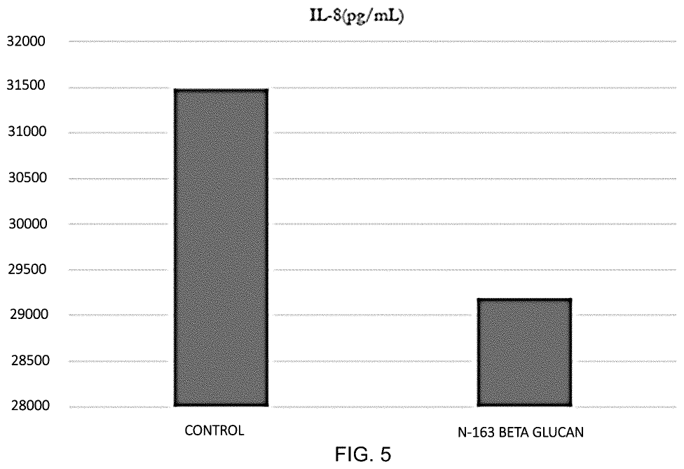

See FIGS. 3-5.
i. IL-6 secretion from immature iPS-dendritic cells (DCs) was 1.07 fold decreased in N-163 stimulated dendritic cells compared to the control.
ii. IL-6 secretion from immature iPS-dendritic cells (DCs) was 2.07 fold decreased in N-163 stimulated dendritic cells compared to the control.
iii. IL-8 secretion was 1.08 fold lesser in N-163 stimulated dendritic cells compared to the control.

### Discussion

Blocking IL-6 treats Liver fibrosis, according to Am J Gastroenterol. 2008 June; 103(6): 1372-9.
Inhibiting IL-6 secretion benefits in inflammation and joint pain, according to Favalli E. G., Rheumatology and Therapy, 7, 473-516 (2020).
Inhibiting IL-6 secretion benefits in aging and chronic diseases, according to Maggio M. et al., J Gerontol A Biol Sci Med Sci, 2006 June; 61(6): 575-584.
Blocking IL-6 is related to therapy for lung fibrosis, according to Papiris S. A. et al., Cytokine. 2018 February; 102:168.
Blocking IL-6 treats kidney fibrosis, according to Chen W, et al., Theranostics. 2019; 9(14): 3980 3991.

IL-6 is the chief stimulator of the production of most acute phase proteins which are associated with at least 25 of the inflammatory disorders (Arthritis Res Ther. 2006; 8 (Suppl 2): S3) and dysregulated continual synthesis of IL-6 plays a pathological effect on chronic inflammation (https://www.ncbi.nlm.nih.gov/pmc/articles/PMC4176007).

Several IL-6 antibodies have been developed as therapeutics against inflammatory disorders and also COVID-19 (https://www.ncbi.nlm.nih.gov/pmc/articles/PMC4176007).

IL-6 drives fibrosis in liver, lung and kidney.

Therefore, reduction of IL-6 by N-163 Beta Glucan can help to combat inflammation and is a potential therapeutic agent against liver fibrosis, lung fibrosis, kidney fibrosis, apart from having potentials in addressing Rheumatoid arthritis and chronic aging related morbidities.
Antibody against IL-8 benefits is shown in Skov L. et al., J Immunol Jul. 1, 2008, 181 (1) 669-679.
Decrease in IL8 levels is related to NASH significance, according to PLOS One. 2011; 6(6): e21381.
Decrease in IL8 levels is related to Pulmonary fibrosis, according to Yang L B. et al., Am J Physiol Lung Cell Mol Physiol. 2018 Jan. 1; 314(1): L127-L136.
Interleukin-8 (IL-8) is a chemoattractant cytokine, especially known as the key mediator associated with inflammation (https://pubmed.ncbi.nlm.nih.gov/8315568/) in neutrophil recruitment and neutrophil degranulation.
Inhibiting the effects of IL-8 signaling is considered to be a potential therapeutic intervention (https://doi.org/10.4061.2011.908468).

IL-6 and IL-8 are key mediator cytokines associated with inflammation. Modulation of IL-6 is beneficial in Liver, Kidney, lung fibrosis and also in rheumatoid diseases as well as chronic age-related morbidities, and IL-8 is linked to progression of liver and lung fibrosis. Therefore, reduction of IL-6 and IL-8 by N-163 Beta Glucan can help to combat inflammation and is a potential therapeutic agent against liver, kidney and lung fibrosis.

### Example 4: F1S Study (Part II)

F1S study data (Part II) show advantages of N-163 & AFO-202 Beta glucans in NASH (Liver disease) & chronic kidney disease.

### Materials and Methods:

Beta Glucan N-163 administered to Kk-Ay mice and compared with control (administration of water for injection) group and AFO-202 group.
The Rats were sacrificed after 28 days.
NEFA levels were analyzed.

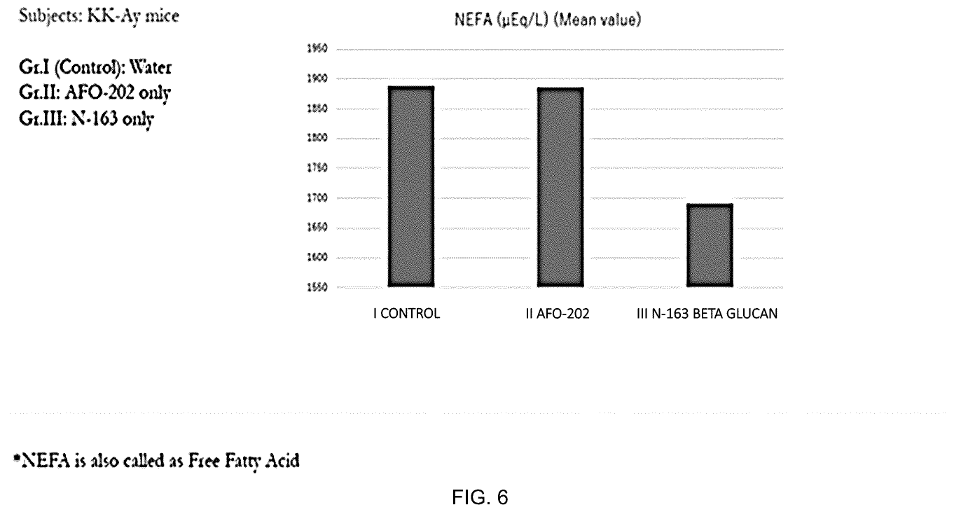

Results: See FIG. 6.

### Discussion

NEFA levels decrease is related to NASH significance, according to Zhang, J W. et al., Scientific Reports volume 4, Article number: 5832 (2014), and Paschos P. et al., Hippokratia. 2009 January March; 13(1): 9 19.
Decrease in NEFA is related to kidney disease, according to Gai Z. B. et al., Nutrients. 2019 April; 11(4): 722.
Effects of AFO-202 Beta Glucan are shown on Lipid levels, according to Vidyasagar Devaprasad Dedeepiya, Case Report, Open Access, Volume 2012, Article ID 895370, and Jegatheesan Saravana Ganesh, J Diet Suppl. J Diet Suppl. 2014 March; 11(1):1-6.

AFO-202 Beta glucan helps decrease lipid levels. Reduction of NEFA by N-163 Beta Glucan which is higher than control and AFO-202 Beta Glucan. Therefore, addition of N-163 Beta Glucan to AFO-202 Beta Glucan can help to combat inflammation, prevent metabolic diseases and has potential in fatty liver disease and chronic kidney disease.

### Example 5: F4S Study (Part II)

F4S study data (Part II) show advantages of combining N-163 & AFO-202 Beta Glucan to prevent and treat fibrosis of Liver, Lung and Kidney diseases and diseases due to vascular calcification.

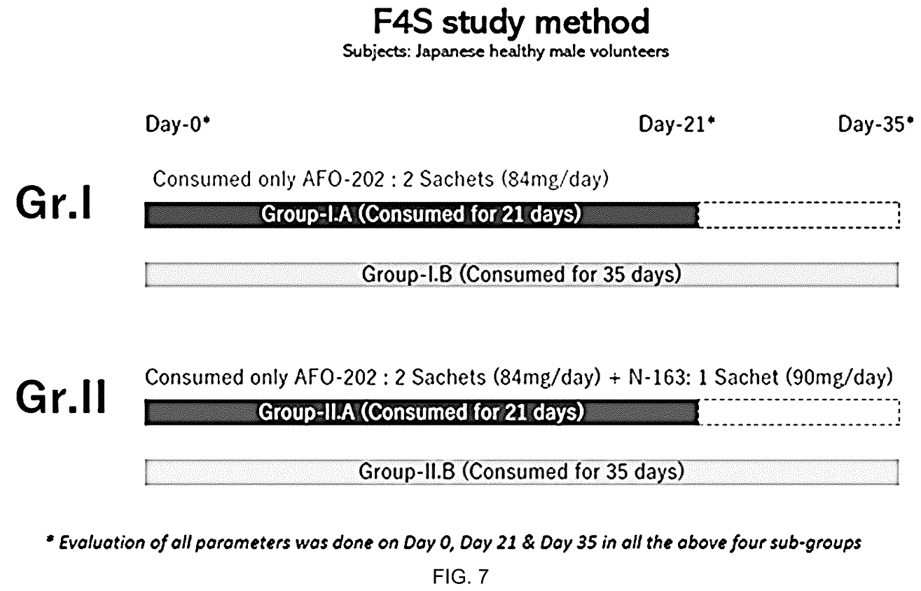

### Materials and Methods: See, FIG. 7.

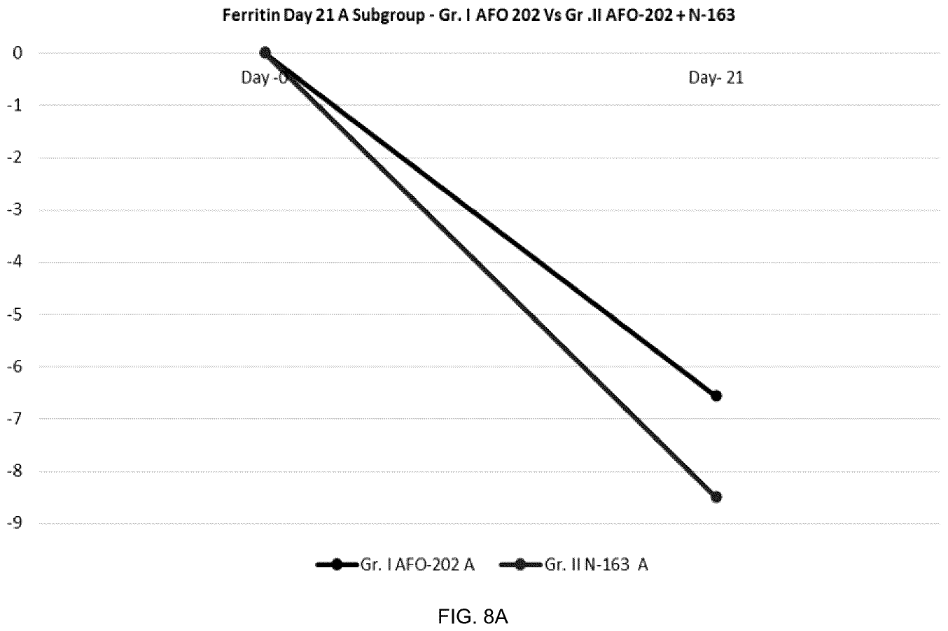

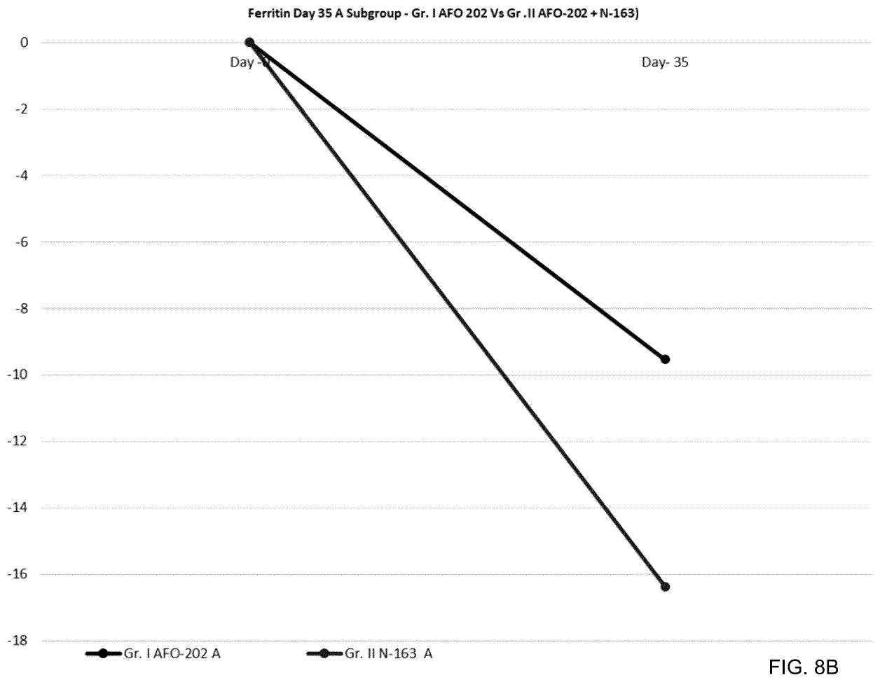

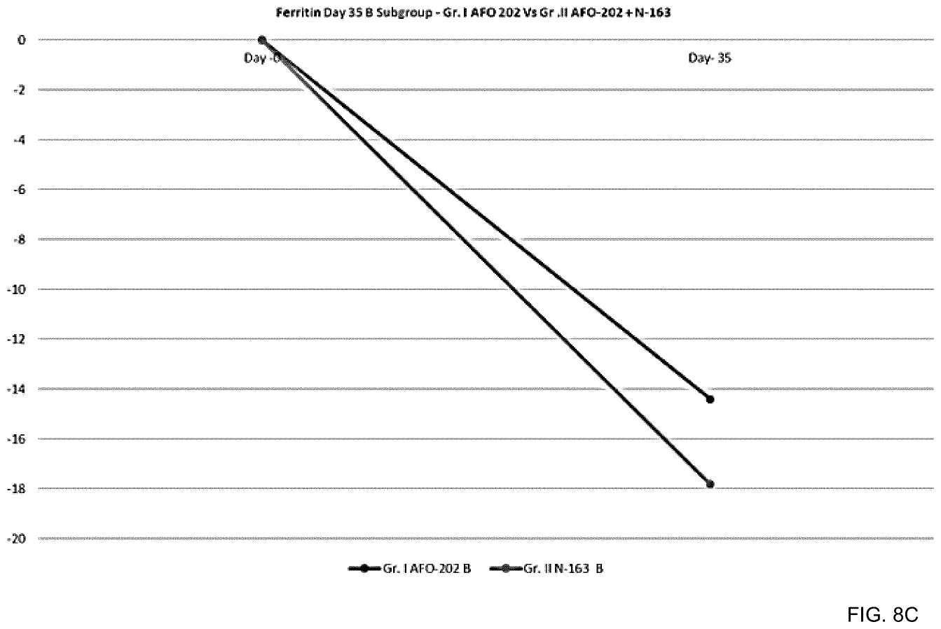

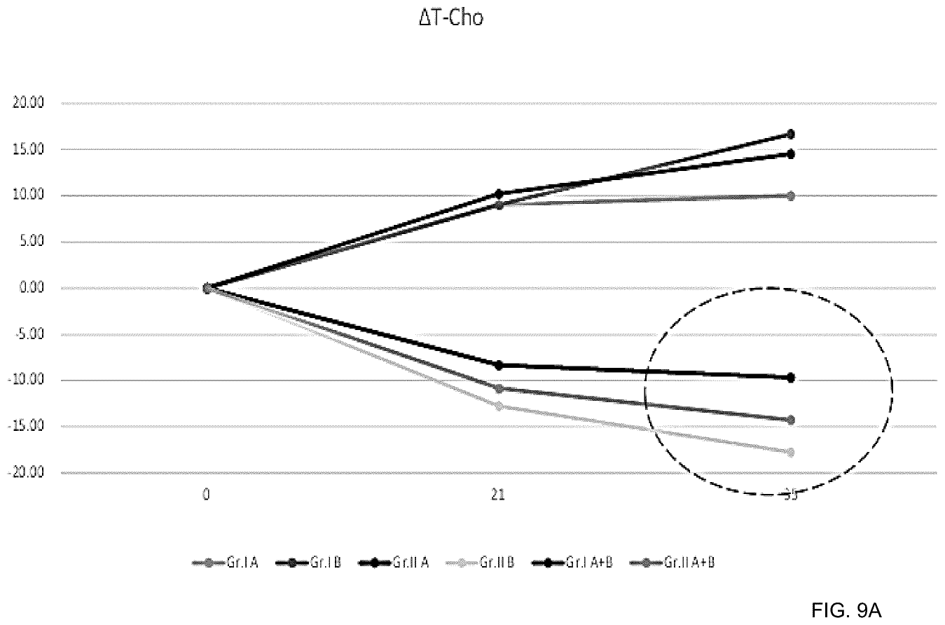

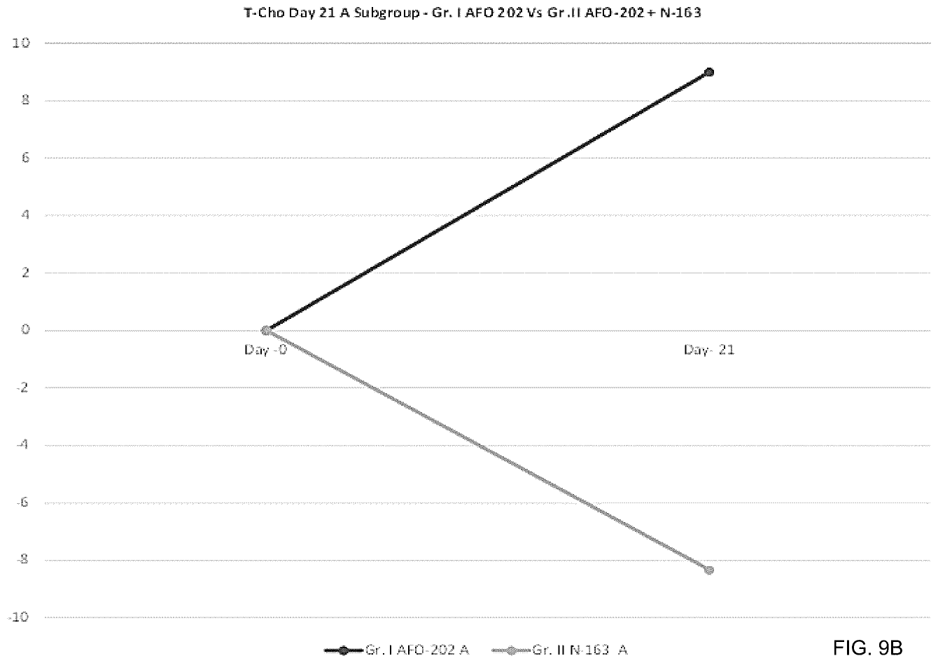

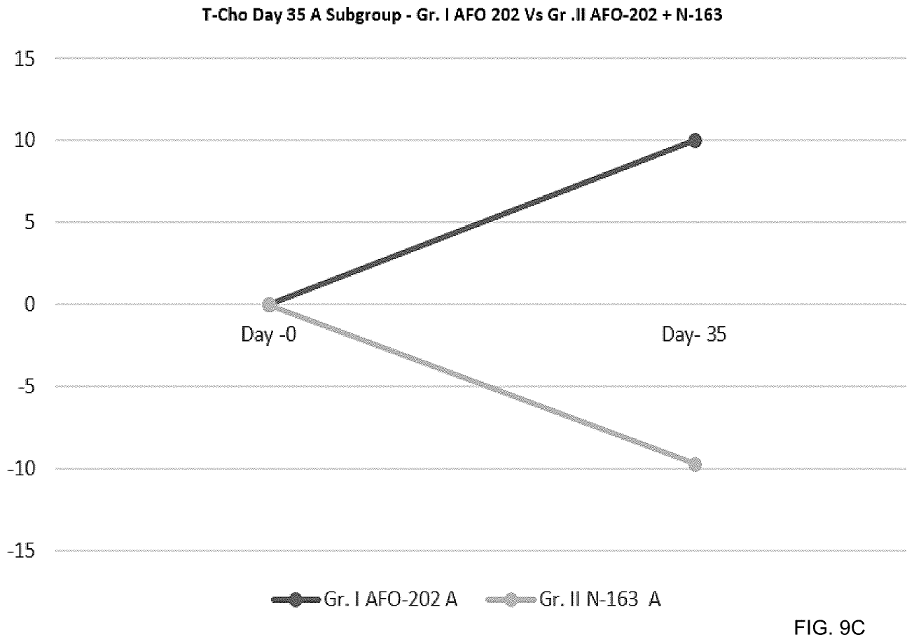

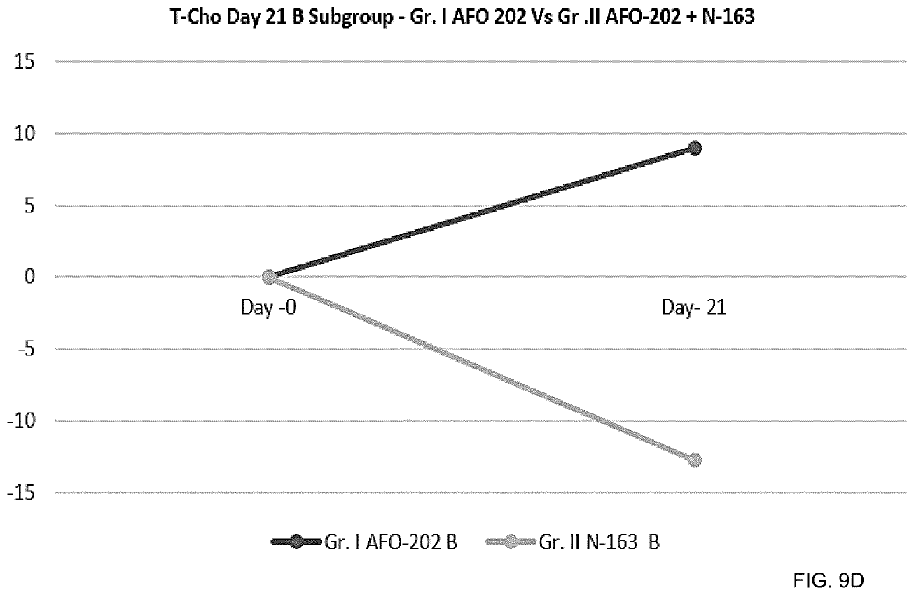

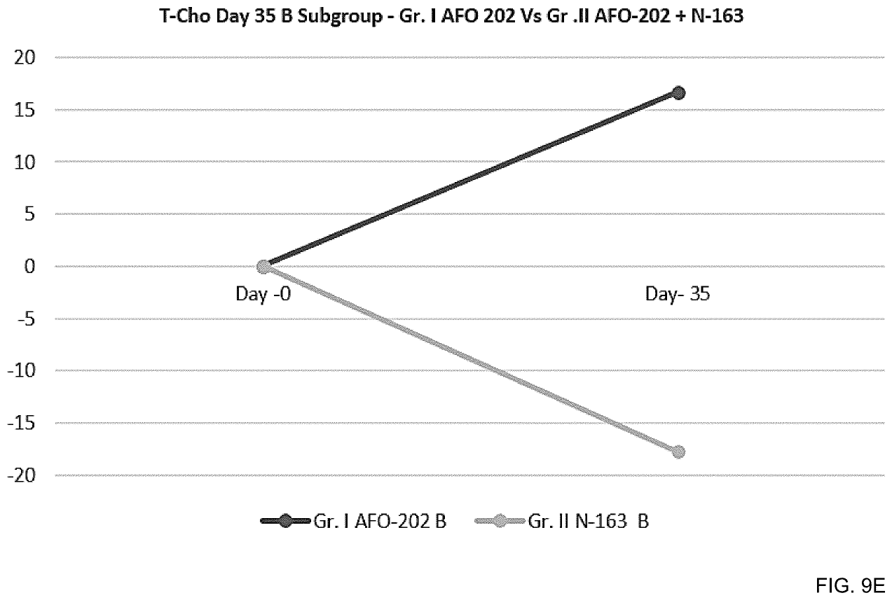

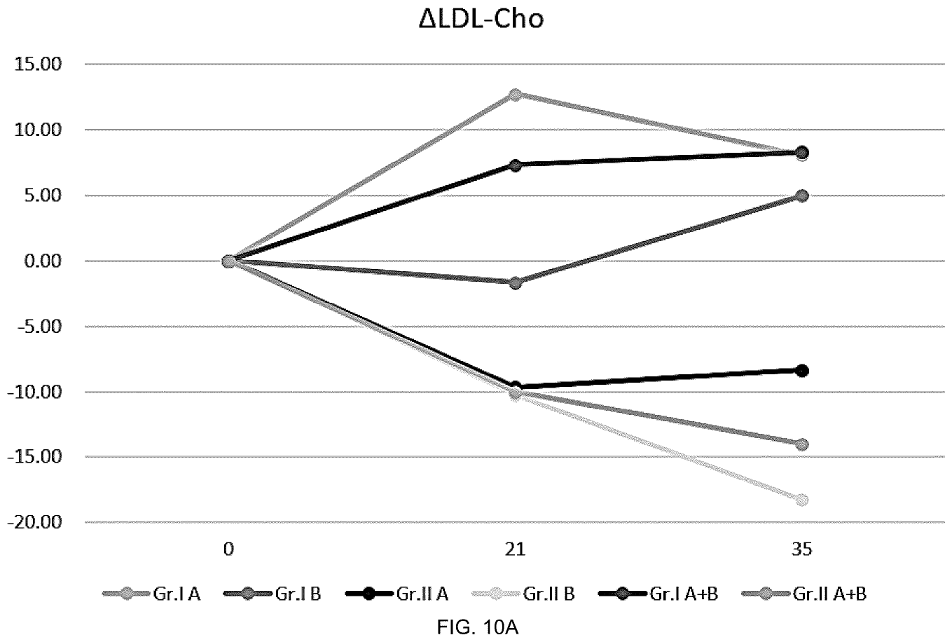

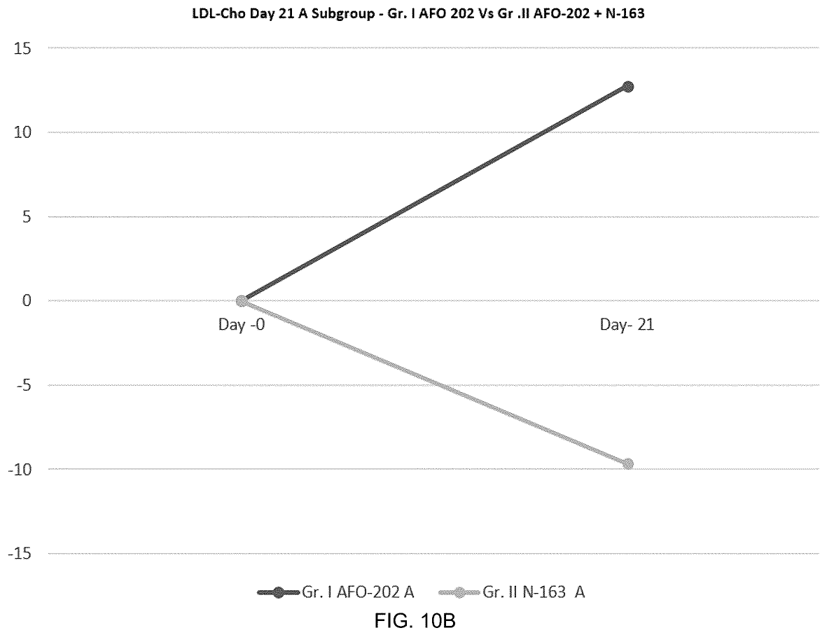

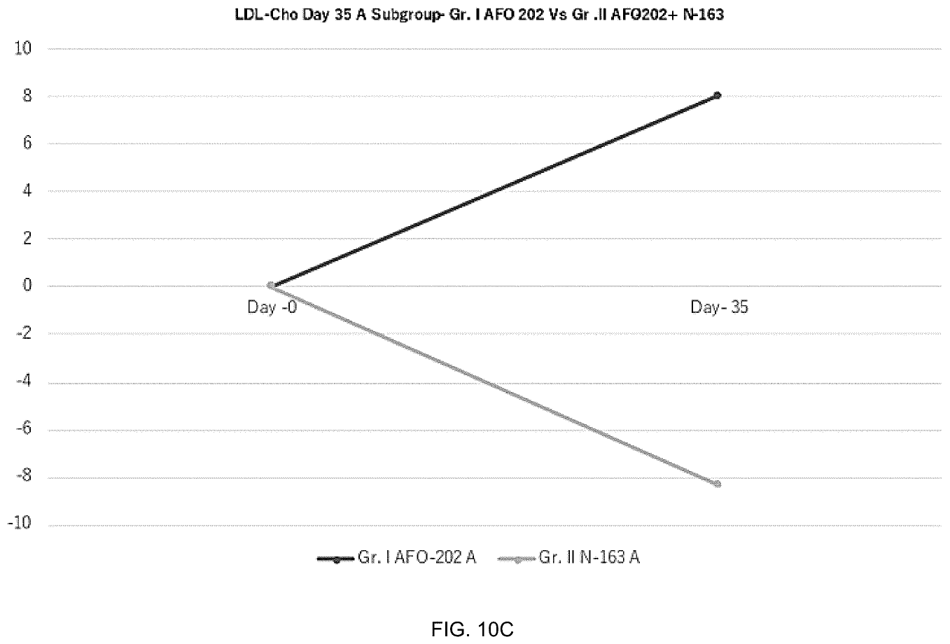

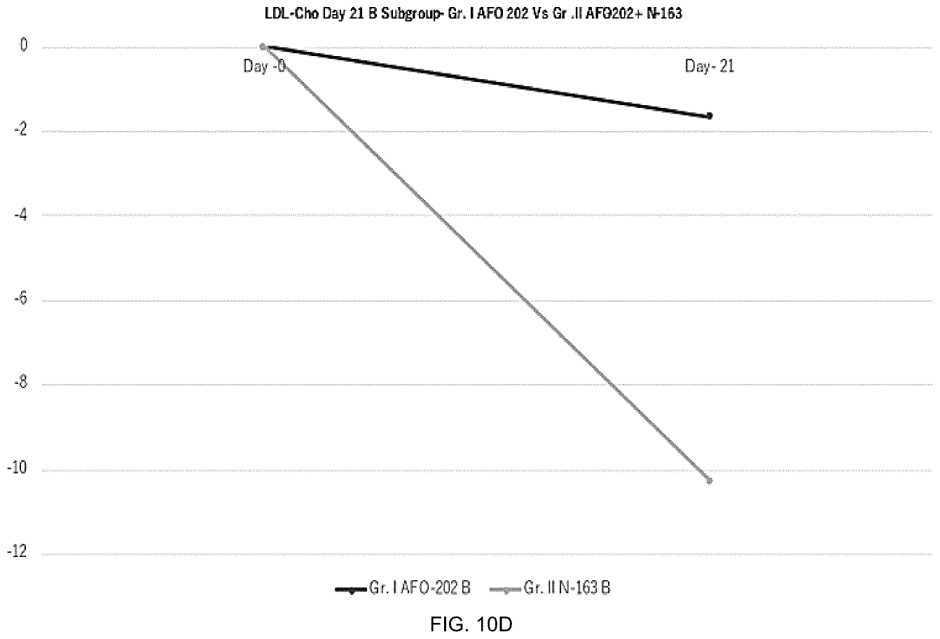

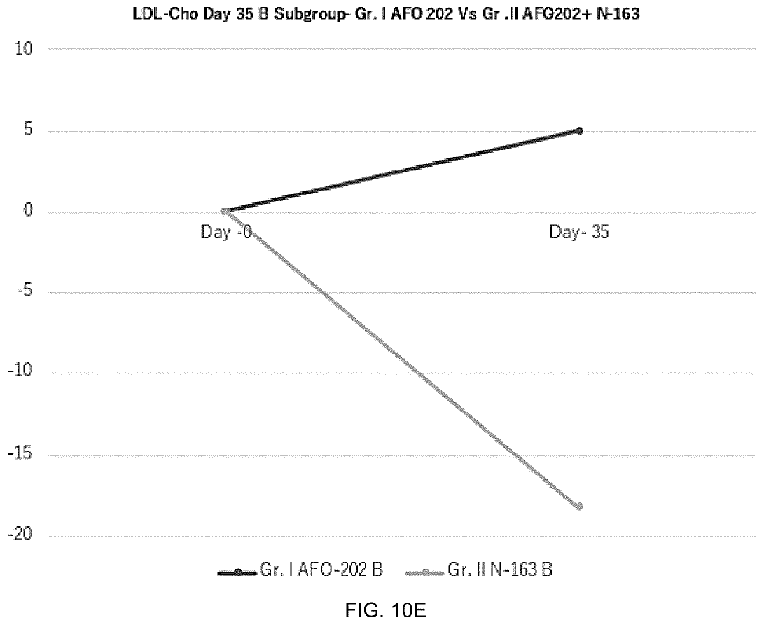

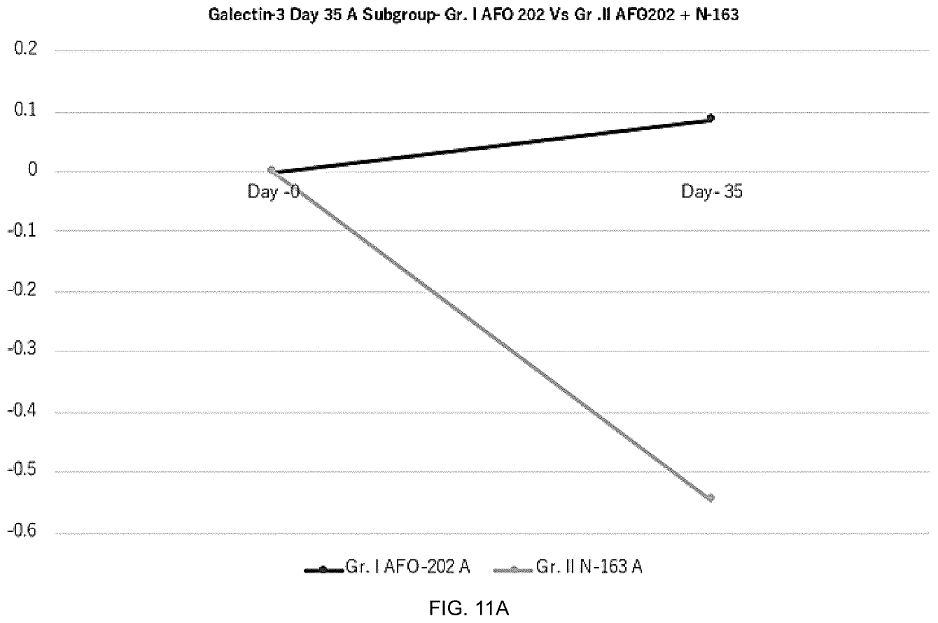

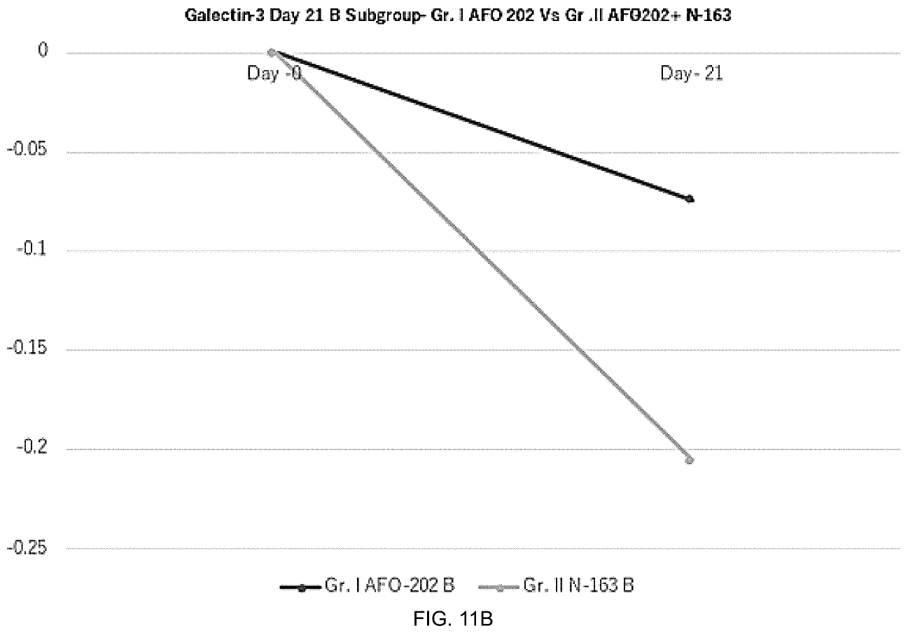

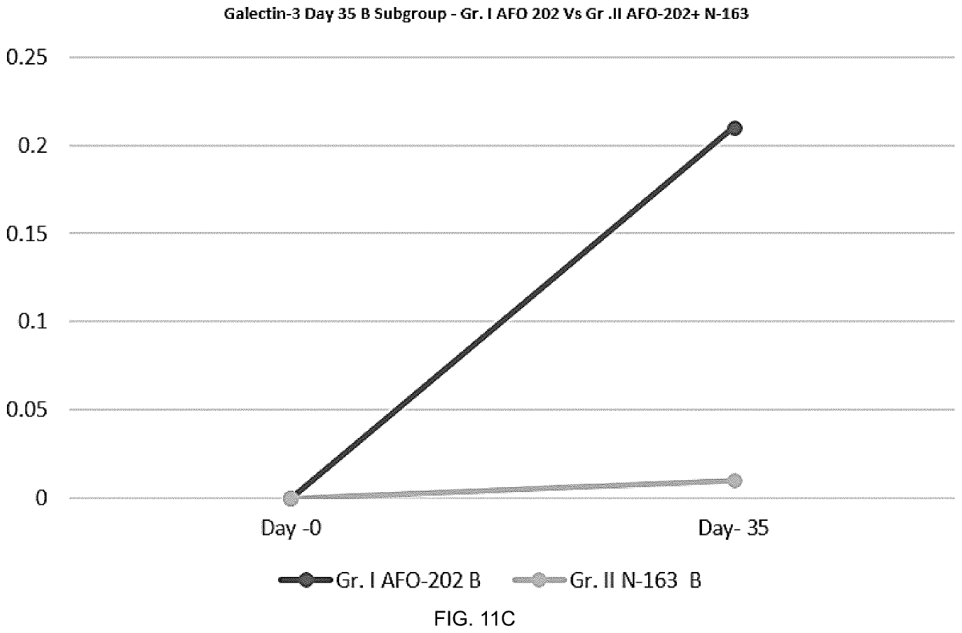

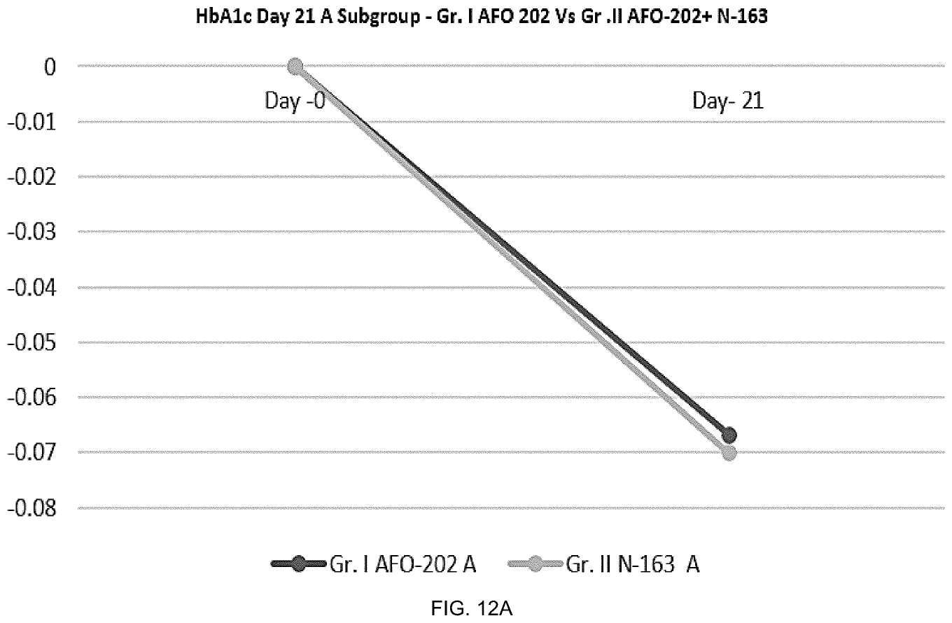

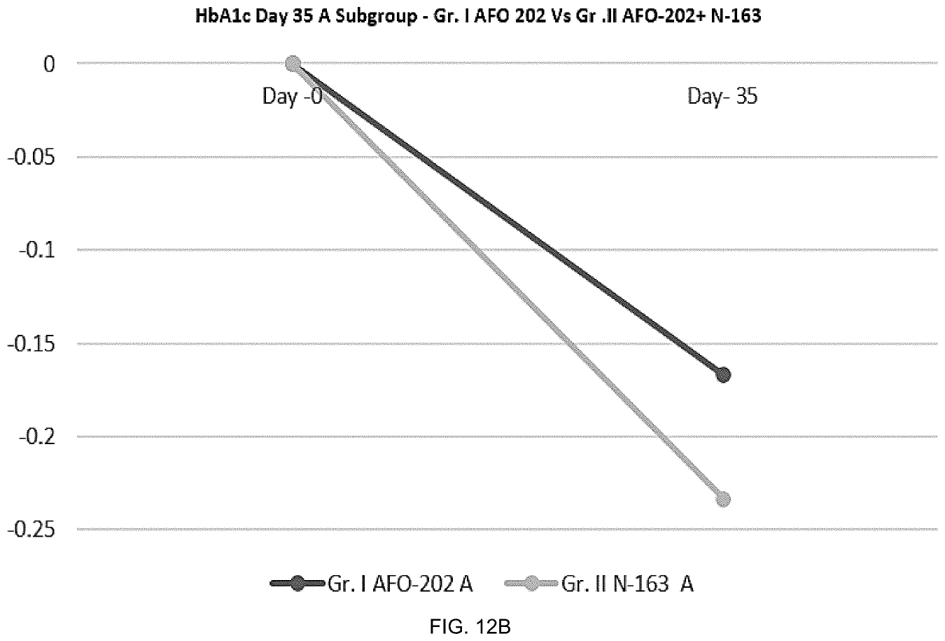

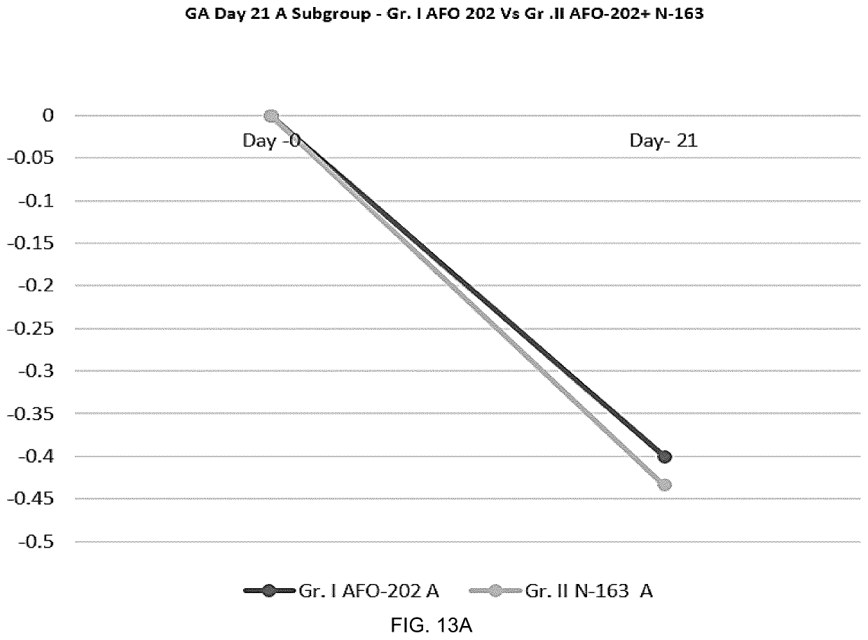

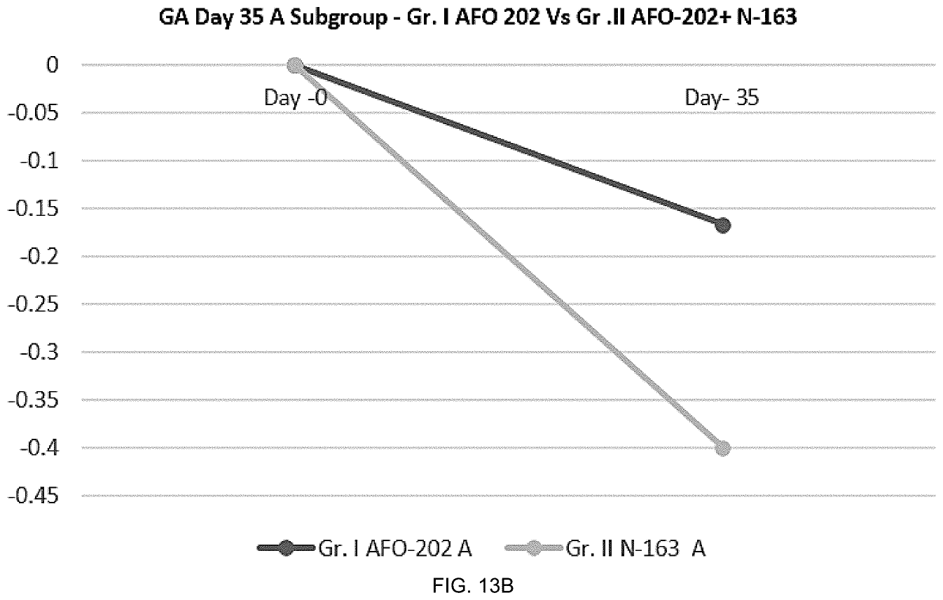

Results: See FIGS. 8-13.

### Discussion

i. Ferritin
Decrease in Ferritin and significance in NASH are shown in Jung J Y. et al., Hepatology International volume 13, pages222-233(2019), and Kowdley K. V. et al., Hepatology. 2012 January; 55(1):77-85.
Decrease in ferritin and significance in lung function are shown in Lee J H. et al., Plos One Published: Apr. 2, 2020 (https://doi.org/10.1371/journal.pone.0231057).
Decrease in Ferritin and significance in Lung fibrosis are shown in Enomoto N. et al., First published: 5 Jun. 2018 https://doi.org/10.1111/crj. 12918.
Decrease in Ferritin and significance in kidney diseases and vascular calcification are shown in Balla J. et al, Pharmaceuticals (Basel). 2019 June; 12(2): 96.

Ferritin is associated with enhancement of liver fibrosis, kidney and lung diseases and also vascular calcification, which leads to aortic and cerebrovascular and coronary artery diseases. Since the combination of AFO-202+N-163 Beta Glucan reduces ferritin levels it holds great potential as a preventive and therapeutic agent for fibrosis of liver, kidney and lung diseases as well as several vascular diseases including cardiac and cerebrovascular diseases.

ii. Total Cholesterol
Decrease in total cholesterol and significance in NASH are shown in Kerr T. A. et al., Hepatology. 2012 November; 56(5): 1995-1998.
Decrease in total cholesterol and significance in CKD are shown in Blood Purif 2018; 46:144-152.

High and dysregulated cholesterol levels is associated with enhancement of liver fibrosis and kidney disease. Since the combination of AFO-202+N-163 Beta Glucan reduces cholesterol levels it holds great potential as a preventive and therapeutic agent for liver and kidney disease.

iii. LDL Cholesterol
Decrease in LDL cholesterol and significance in NASH are shown in Chatrath H. et al., Semin Liver Dis. 2012 February; 32(1): 22-29.
Decrease in LDL cholesterol and significance in CKD are shown in Haynes R. et al, JASN August 2014, 25 (8) 1825-1833.

High and dysregulated LDL cholesterol levels is associated with enhancement of liver fibrosis and kidney disease. Since the combination of AFO-202+N-163 Beta Glucan reduces cholesterol levels it holds great potential as a preventive and therapeutic agent for liver and kidney disease.

iv. Galectin-3
Decrease in Galectin-3 and significance in NASH are shown in Clinical Trial Gastroenterology. 2020 April; 158(5): 1334-1345.e5.

High and dysregulated Galectin-3 levels is associated with enhancement of liver fibrosis disease. Since the combination of AFO-202+N-163 Beta Glucan reduces cholesterol levels it holds great potential as a preventive and therapeutic agent for fatty liver disease.

v. HbA1c and Glycated Albumin (GA)
Glycated albumin/HbA1c and significance in NASH are shown in Diabetes Res Clin Pract. 2017 March; 125:53-61.

High HbA1C and GA levels are associated with enhancement of liver fibrosis disease. Since the combination of AFO-202+N-163 Beta Glucan reduces HbA1C and GA levels it holds great potential as a preventive and therapeutic agent for fatty liver disease.

### Example 6: F18S Study

### Materials and Methods:

Eight STAM mice were included in each study group. There were five study groups, described below.

### Study Groups:

### Group 1: Vehicle

Eight NASH mice were orally administered the vehicle (reverse osmosis [RO] water) in a volume of 5 mL/kg once daily from 6 to 9 weeks of age.

### Group 2: AFO-202 Beta Glucan

Eight NASH mice were orally administered the vehicle supplemented with AFO-202 beta glucan at a dose of 1 mg/kg in a volume of 5 mL/kg once daily from 6 to 9 weeks of age.

### Group 3: N-163 Beta Glucan

Eight NASH mice were orally administered the vehicle supplemented with N-163 beta glucan at a dose of 1 mg/kg in a volume of 5 mL/kg once daily from 6 to 9 weeks of age.

### Group 4: AFO-202 Beta Glucan+N-163 Beta Glucan

Eight NASH mice were orally administered the vehicle supplemented with AFO-202 beta glucan at a dose of 1 mg/kg in a volume of 5 mL/kg once daily and orally administered the vehicle supplemented with N-163 beta glucan at a dose of 1 mg/kg in a volume of 5 mL/kg once daily from 6 to 9 weeks of age.

### Group 5: Telmisartan

Eight NASH mice were orally administered the vehicle supplemented with telmisartan at a dose of 10 mg/kg once daily from 6 to 9 weeks of age.

### Test Substances

AFO-202 beta glucan and B were provided by GN Corporation Co Ltd. Telmisartan (Micardis®) was purchased from Boehringer Ingelheim GmbH (Germany).

### Instruction for the Preparation of Test Substances

AFO-202 Beta Glucan and N-163 Beta Glucan

AFO-202 beta glucan or N-163 beta glucan was mixed in the required amount of RO water and stirred until it completely dissolved. The solution was dispensed into 7 tubes and stored at 4° C. until the day of administration. The dosing formulations were stirred prior to administration. The dosing formulations were used within 7 days.

### Telmisartan

Formulations were freshly prepared prior to administration. One tablet of telmisartan was transferred into mortar and triturated using a pestle by adding RO water gradually to get 1 mg/ml of homogeneous suspension.

TABLE 1
Study design and treatment schedule

| | No. | | Test | Dose | Volume | | |
|---|---|---|---|---|---|---|---|
| Group | mice | Mice | substance | (mg/kg) | (mL/kg) | Regimen | Sacrifice |
| 1 | 8 | STAM | Vehicle | — | 5 | PO, QD, | End of the |
| | | | | | | 6th to 9^{th} week | 9th week |
| | | | | | | (21 days) | |
| 2 | 8 | STAM | AFO-202 | 1 | 5 | PO, QD, | End of the |
| | | | Beta Glucan | | | 6th to 9^{th} week | 9th week |
| | | | | | | (21 days) | |
| 3 | 8 | STAM | N-163 | 1 | 5 | PO, QD, | End of the |
| | | | Beta Glucan | | | 6th to 9^{th} week | 9th week |
| | | | | | | (21 days) | |
| 4 | 8 | STAM | AFO-202 | 1 | 5 | PO, QD, | End of the |
| | | | Beta Glucan | | | 6th to 9^{th} week | 9th week |
| | | | N-163 | 1 | | (21 days) | |
| | | | Beta Glucan | | | | |
| 5 | 8 | STAM | Telmisartan | 10 | 5 | PO, QD, | End of the |
| | | | | | | 6th to 9^{th} week | 9th week |
| | | | | | | (21 days) | |

### NASH Induction

NASH was induced in male mice by a single subcutaneous injection of 200 μg streptozotocin (STZ, Sigma-Aldrich, USA) solution 2 days after birth and feeding with a high-fat diet (HFD, 57 kcal % fat, Cat #HFD32, CLEA Japan, Inc., Japan) after 4 weeks of age.

### Drug Administration Route

The vehicle, AFO-202 beta glucan, N-163 beta glucan, and telmisartan were administered orally in a volume of 5 ml/kg.

### Treatment Doses

1) AFO-202 beta glucan was administered at a dose level of 1 mg/kg once daily.
2)N-163 beta glucan was administered at a dose level of 1 mg/kg once daily.
3) Telmisartan was administered at a dose level of 10 mg/kg once daily.

### Animals

C57BL/6J mice (14-day-pregnant female) mice were obtained from Japan SLC, Inc. (Japan). NASH was induced in male mice (offspring from the pregnant mice) by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Inc., Japan) after 4 weeks of age. All animals used in this study were cared for under the following guidelines:
1) Act on Welfare and Management of Animals
(Ministry of the Environment, Japan, Act No. 105 of Oct. 1, 1973)
2) Standards Relating to the Care and Management of Laboratory Animals and Relief of Pain
(Notice No.88 of the Ministry of the Environment, Japan, Apr. 28, 2006)
3) Guidelines for Proper Conduct of Animal Experiments
(Science Council of Japan, Jun. 1, 2006)

### Environment

The animals were maintained in a specific pathogen-free (SPF) facility under controlled conditions of temperature (23±3° C.), humidity (50±20%), lighting (12-hour artificial light and dark cycles; light from 8:00 to 20:00) and air exchange.

### Animal Husbandry

The animals were housed in TPX™ cages (CLEA Japan), with a maximum of 4 mice per cage. Sterilized Pulmas™ (Material Research Center Co., Ltd, Japan) was used for bedding and replaced once a week.

### Food and Drink

A sterilized solid HFD was provided ad libitum, placed in a metal lid on the top of the cage. RO water was provided ad libitum from a water bottle equipped with a rubber stopper and a sipper tube. Water bottles were replaced once a week, cleaned, sterilized in an autoclave and reused.

### Animal and Cage Identification

Mice were identified by ear punch. Each cage was labelled with a specific identification code.

### Randomization

NASH model mice were randomized into five groups of eight mice at six weeks of age based on their body weight the day before the start of treatment. The randomization was performed by body weight-stratified random sampling using Microsoft Excel software. NASH model mice were stratified by their body weight to get the SD and difference in the mean weights among groups as small as possible.

### Animal Monitoring and Sacrifice

The mice's viability, clinical signs (lethargy, twitching, laboured breathing) and behaviour were monitored daily. Body weight was recorded daily before the treatment. Mice were observed for significant clinical signs of toxicity, moribundity and mortality before and after administration. The animals were sacrificed at 9 weeks of age by exsanguination through direct cardiac puncture under isoflurane anaesthesia (Pfizer Inc.).

If an animal showed >25% body weight loss within a week or >20% body weight loss compared with the previous day, the animal was euthanized ahead of study termination, and samples were not collected. If it showed a moribundity sign, such as prone position, the animal was euthanized ahead of study termination, and samples were not collected.

### Sample Collection

The following samples were collected and stored.
Frozen plasma samples
Frozen liver samples
Paraffin-embedded liver blocks
OCT-embedded liver blocks

### Preparation of Plasma Samples:

At study termination, non-fasting blood was collected through direct cardiac puncture using pre-cooled syringes. The collected blood was transferred in pre-cooled polypropylene tubes with anticoagulant (Novo-Heparin) and stored on ice until centrifugation. The blood samples were centrifuged at 1,000×g for 15 minutes at 4° C. The supernatant was collected and stored at −80° C. for biochemistry and evaluation.

### Preparation of Liver Samples:

After sacrifice, the whole liver was collected and washed with cold saline. Photos of individual whole livers (parietal side and visceral side) were taken. Liver weight was measured, and liver-to-body weight ratio was calculated. The left lateral lobes of the livers were separated, dissected and stored.
A: Liver specimens were stored at −80° C. embedded in optimal cutting temperature (OCT, Sakura Finetek Japan, Japan) compound for immunohistochemistry.
B: Liver specimens were fixed in Bouin's solution (Sigma-Aldrich Japan, Japan) for 24 hours. After fixation, these specimens were proceeded to paraffin embedding for HE and Sirius red staining.
C: Liver specimens were snap frozen in liquid nitrogen and stored at −80° C. for further analysis.

The left and right medial lobes were snap frozen in liquid nitrogen and stored at −80° C. for evaluation.

The right lobe was snap frozen in liquid nitrogen and stored at −80° C. for biochemistry analysis.

The caudate lobe was snap frozen in liquid nitrogen and stored at −80° C. for evaluation.

### Measurement of Plasma Biochemistry

Plasma ALT levels were measured by FUJI DRI-CHEM 7000 (Fujifilm Corporation).

### Measurement of Liver Biochemistry

### Measurement of Liver Lipid Content

Liver total lipid extracts were obtained by Folch's method (Folch J. et al., J. Biol. Chem. 1957; 226: 497). Liver samples were homogenized in chloroform-methanol (2:1, v/v) and incubated overnight at room temperature. After washing with chloroform-methanol-water (8:4:3, v/v/v), the extracts were evaporated to dryness and dissolved in isopropanol. Liver triglyceride content was measured by the Triglyceride E-test (Wako Pure Chemical Industries, Ltd., Japan). Liver free fatty acid content was measured by the NEFA C-test (FUJIFILM Wako Pure Chemical Corporation).

### Blinding for Histological Analysis

Sections were cut from paraffin blocks of liver tissue using a rotary microtome (Leica Microsystems). After sectioning, each slide was coded with a number for blind evaluation. Each number was generated using the RAND function of Excel software, sorted in ascending order and assigned to slides. The tissue slides were used for the following stains and evaluated by an experimenter.

### Histological Analyses

For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). The NAFLD Activity Score (NAS) was calculated according to the criteria of Kleiner (Kleiner D E. Et al., Hepatology, 2005; 41:1313), as shown in Table 2. For NAS scoring, bright field images of HE-stained sections were captured using a digital camera (DFC295; Leica, Germany) at 50- and 200-fold magnifications. Steatosis score in 1 section/mouse (representative 1 field at 50-fold magnification), inflammation score in 1 section/mouse (representative 1 field around the central vein at 200-fold magnification) and ballooning score in 1 section/mouse (representative 1 field around the central vein at 200-fold magnification) were estimated.

TABLE 2

| | | |
|---|---|---|
| Item | Extent | Score |

| | | |
|---|---|---|
| | Steatosis at 50-fold magnification | |
| Steatosis | <5% | 0 |
| | 5-33% | 1 |
| | >33-66% | 2 |
| | >66% | 3 |
| | Estimation of inflammatory foci | |
| Lobular | No foci | 0 |

| | | | |
|---|---|---|---|
| inflammation | <2 | foci/200x | 1 |
| | 2-4 | foci/200x | 2 |
| | >4 | foci/200x | 3 |

| | | |
|---|---|---|
| | Estimation of number of ballooning cells | |
| Ballooning | None | 0 |
| | Few ballooning cells | 1 |
| | Many cells/prominent ballooning | 2 |

To visualize collagen deposition, Bouin's fixed liver sections were stained using picro-Sirius red solution (Waldeck, Germany). Briefly, sections were deparaffinized and hydrophilized with xylene, 100-70% alcohol series and RO water, and then treated with 0.03% picro-Sirius red solution (Cat No.: 1A-280) for 60 minutes. After washing with 0.5% acetic acid solution and RO water, stained sections were dehydrated and cleared with 70-100% alcohol series and xylene, then sealed with Entellan® new (Merck, Germany) and used for observation.

For immunohistochemistry, sections were cut from frozen liver tissues embedded in Tissue-Tek OCT compound and fixed in acetone. Endogenous peroxidase activity was blocked using 0.03% H₂O₂ for 5 minutes, followed by incubation with Block Ace (Dainippon Sumitomo Pharma Co. Ltd., Japan) for 10 minutes. The sections were incubated with anti-F4/80 antibody at 4° C. overnight. After incubation with a secondary antibody, enzyme-substrate reactions were performed using 3, 3′-diaminobenzidine/H₂O₂ solution (Nichirei Bioscience Inc., Japan). Profiles of primary and secondary antibodies are shown in Table 3.

TABLE 3
Profile of primary and secondary antibodies
for immunochemical staining

| Test | Details of primary | Details of secondary |
|---|---|---|
| antibody | antibody | antibody |
| F4/80 | Name: Monoclonal | Name: VECTASTAIN ABC |
| | Antibody | KIT |
| | To Mouse Macrophages | Manufacturer: Vector |
| | | Laboratories |
| | Manufacturer: BMA | Lot #: TBD |
| | Biomedicals | Cat #: PK-4004 |
| | Lot #: TBD | |
| | Cat #: T-2006 | |
| | Dilution: 100 fold | |

For quantitative analysis of the fibrosis area and inflammation area, bright field images of Sirius red-stained and F4/80-immunostained sections were captured around the central vein using a digital camera (DFC295; Leica, Germany) at 200-fold magnification, and the positive areas in 5 fields/section were measured using ImageJ software (National Institute of Health, USA).

### Statistical Tests

Statistical analyses were performed using Prism Software 6 (GraphPad Software, USA). Statistical analyses were conducted using the Bonferroni multiple comparison test. Comparisons were made between the following groups:
1) Group 1 (Vehicle) vs. Group 2 (AFO-202 Beta Glucan), Group 3 (N-163 Beta Glucan), Group 4 (AFO-202 Beta Glucan+B) and Group 5 (Telmisartan)
P values <0.05 were considered statistically significant. Results were expressed as mean+SD.

A trend or tendency was assumed when a one-sided t-test returned P values <0.1. Comparisons were made between the following groups:
2) Group 1 (Vehicle) vs. Group 2 (AFO-202 Beta Glucan)
3) Group 1 (Vehicle) vs. Group 3 (N-163 Beta Glucan)
4) Group 1 (Vehicle) vs. Group 4 (AFO-202 Beta Glucan+B)
5) Group 1 (Vehicle) vs. Group 5 (Telmisartan)

### Results:

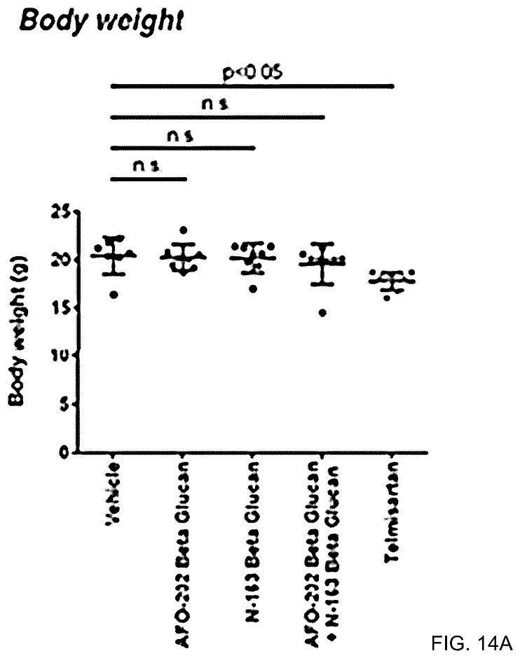

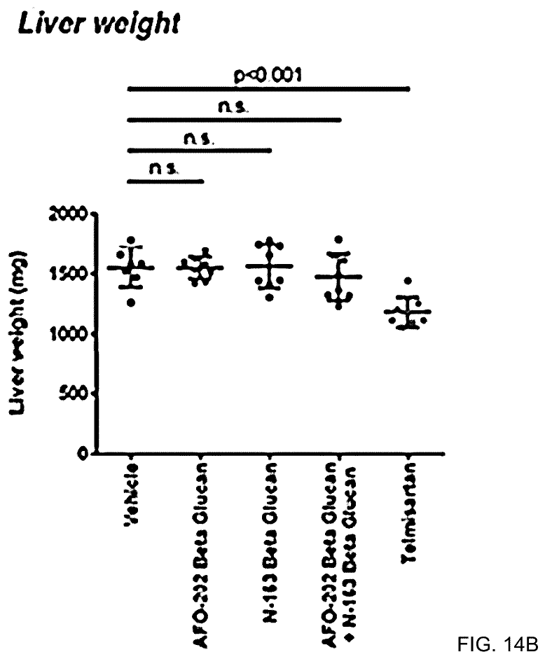

There was so significant difference in body weight and liver weight between the groups (FIG. 14). The mean liver weight was 20.4 g in Group 1, 20.3 g in Group 2, 20.2 g in Group 3, 20 g in Group 4 and 17.8 g in Group 5. The mean body weight was 1552 mg in Group 1, 1552 mg in Group 2, 1565 mg in Group 3, 1474 mg in Group 4 and 1181 mg in Group 5.

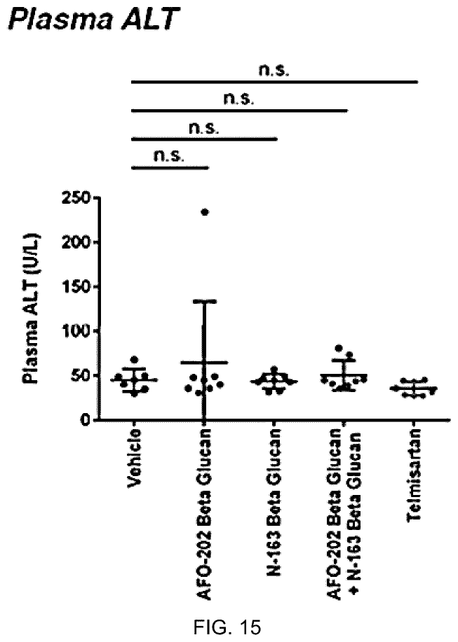

Plasma ALT levels were lowest in the telmisartan group (Mean=36 U/L), followed by Group 3 (Mean=44 U/L) (FIG. 15).

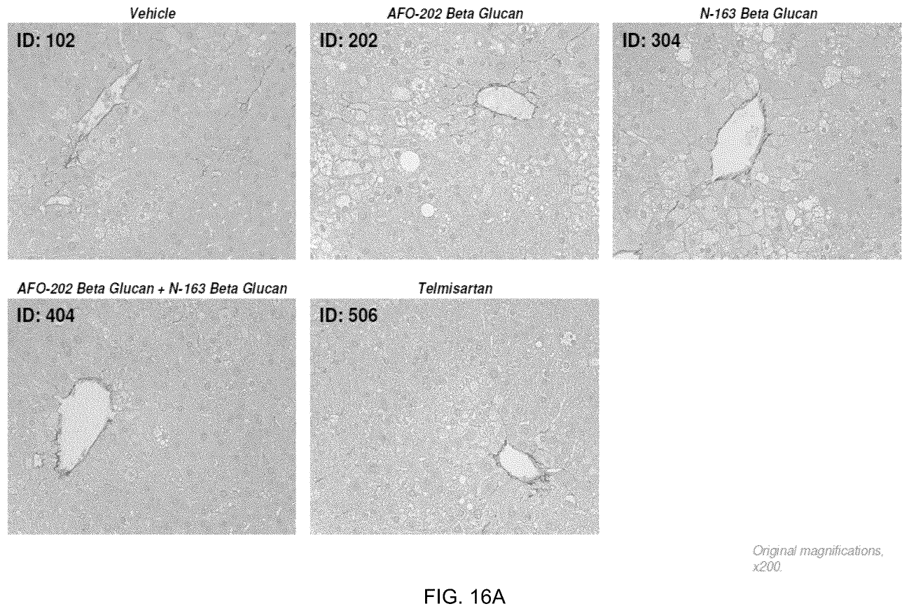

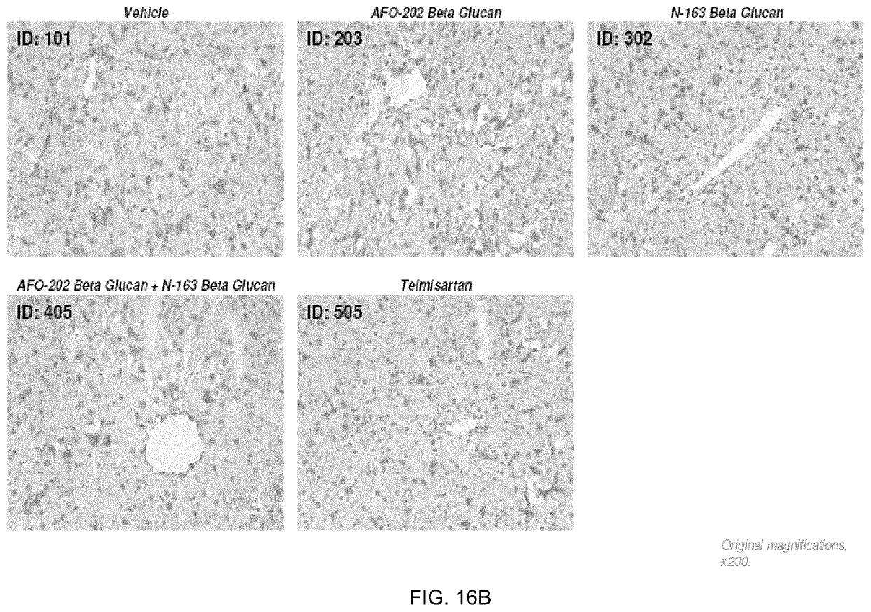

Sirius red-stained images to assess liver damage showed significantly decreased positive staining area in the AFO-202+N-163 and N-163 groups compared with all the other groups (average positive stained area, AFO-202-0.72 AFO-202+N-163: 0.54; N-163: 0.56; telmisartan: 0.59; and vehicle: 0.84) (FIG. 16A).

Regarding NAS, the telmisartan, N-163 and AFO-202+N-163 groups presented a significantly lesser score (average score, telmisartan: 2.625; AFO-202+N-163: 3.25), with lesser micro- and macro-vesicular fat deposition, lobular inflammatory cell infiltrate, and hepatocellular ballooning compared with the other groups; N-163: 3.5; AFO-202: 3.25 and vehicle: 4.571).

The inflammation score was significantly decreased in the AFO-202+N-163 and

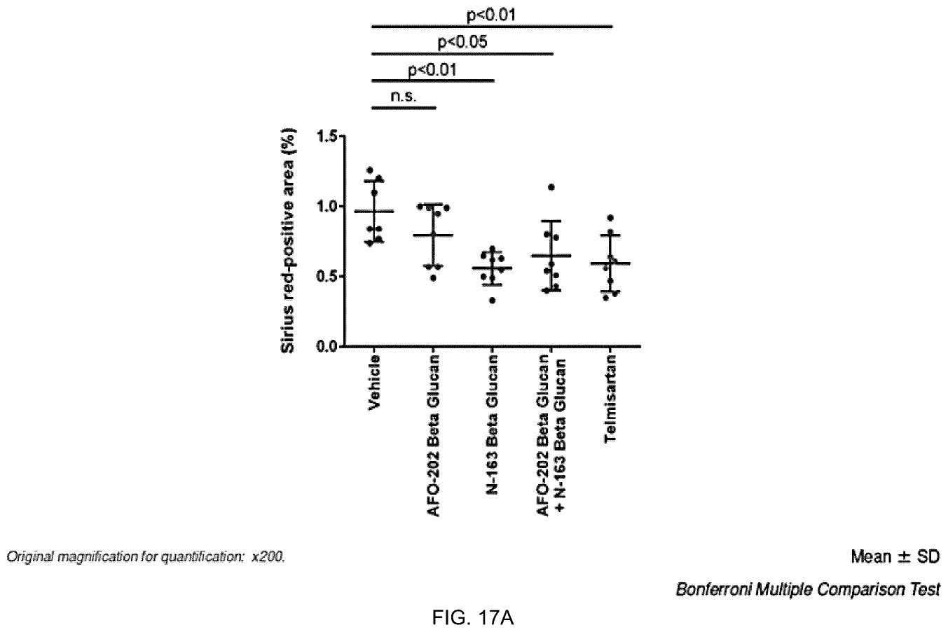

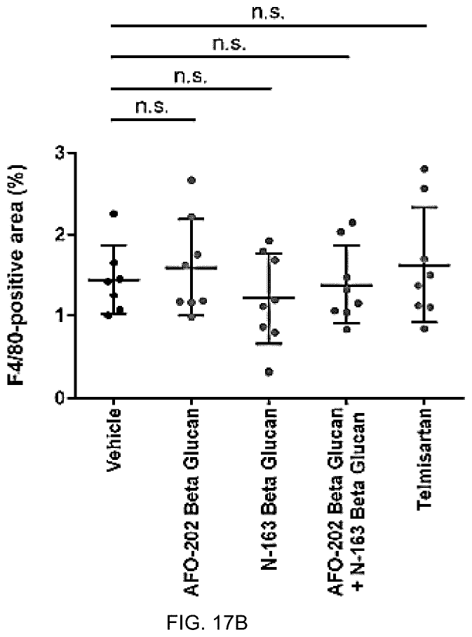

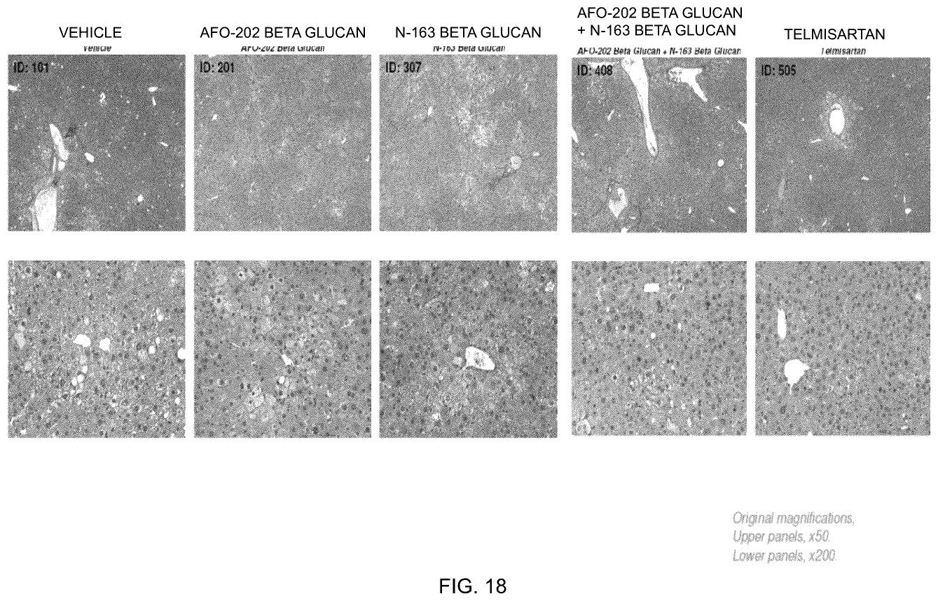

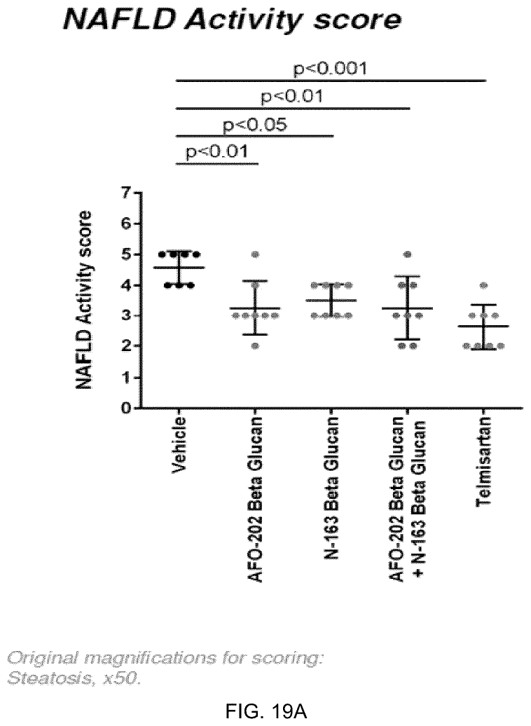

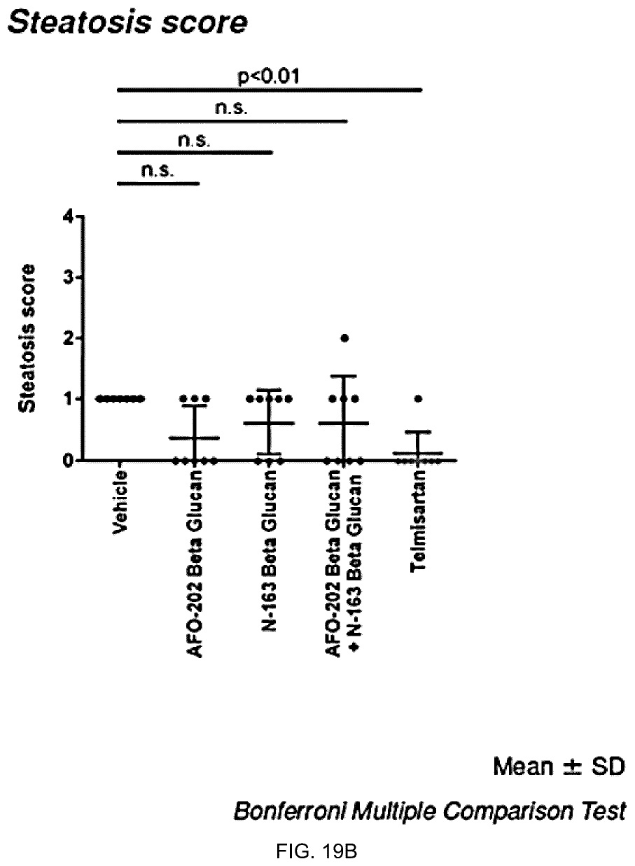

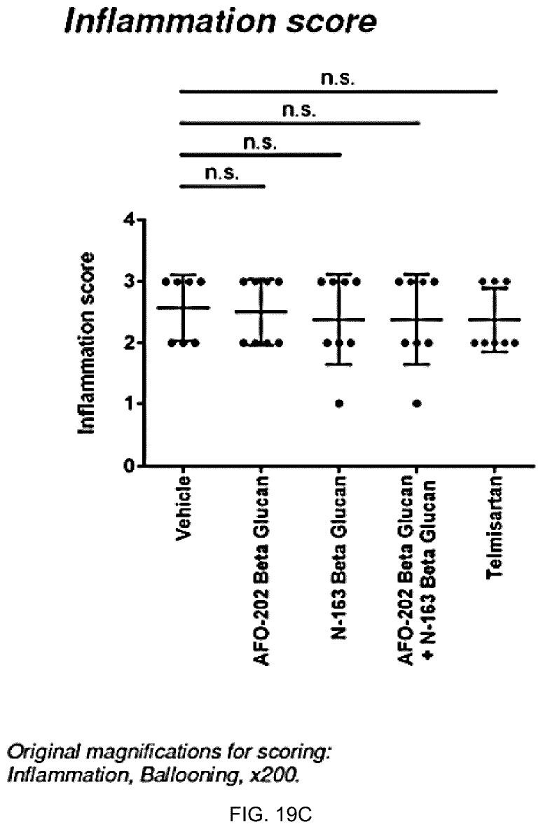

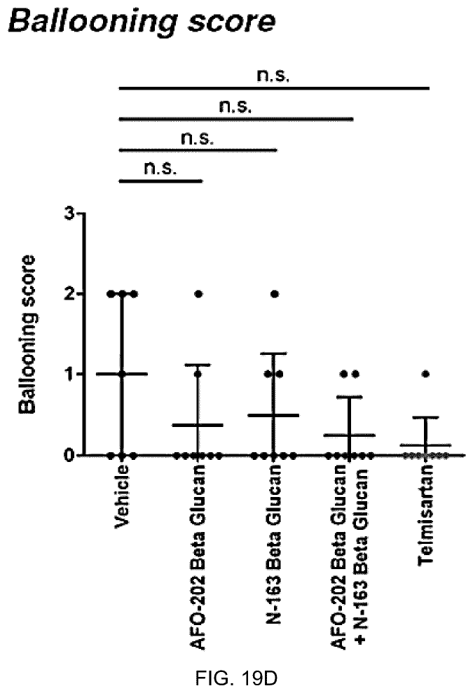

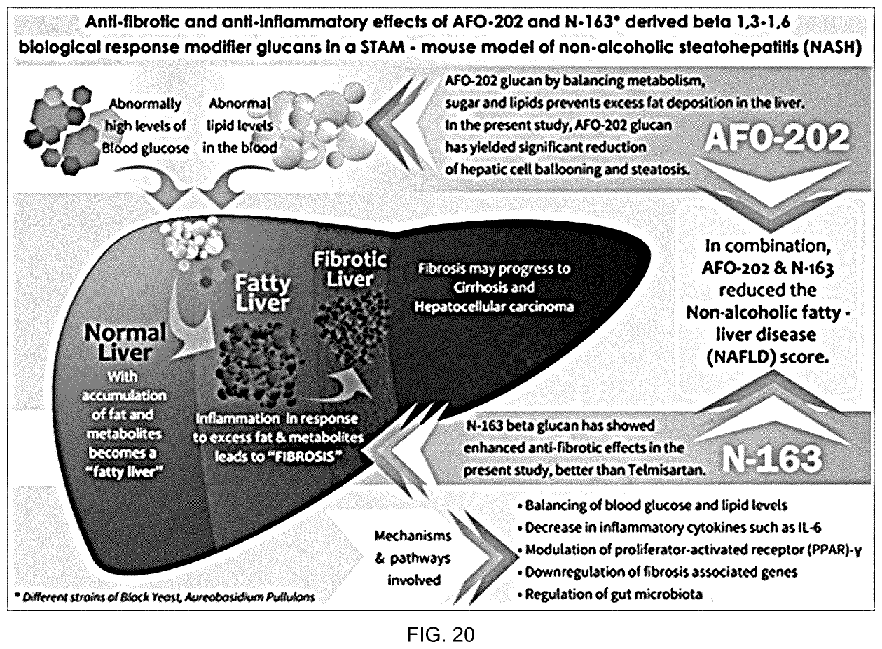

N-163 groups compared with the telmisartan group (FIGS. 17A, 17B, 18). Ballooning and steatosis score was decreased most in the telmisartan group, but a considerable decrease compared with the vehicle was observed in the AFO-202 beta glucan groups (FIG. 19).

The F4/80 immunostaining showed that, the infiltration of macrophages was greatly decreased in the N-163 group indicating decreased inflammation compared to Vehicle and Telmisartan groups. N-163 helps to ameliorate inflammation-fibrosis in STAM model of NASH. N-163 Beta glucan can be a potential agent for ameliorating organ fibrosis in liver, kidney, lung and other organ fibrosis.

N-163 Beta Glucan has the following effects on biomarkers related to NASH and hence adding it to AFO-202 beta Glucan is advantageous:
1. Increased CD11b compared to AFO-202 Beta Glucan; CD11b positive cells are needed for amelioration of liver fibrosis (ref: https://pubmed.ncbi.nlm.nih.gov/22544759/) —Liver fibrosis
2. Decreased Ferritin compared to AFO-202 Beta Glucan; Increase in ferritin is associated with increased liver fibrosis (ref: https://doi.org/10.1007/s12072-018-9892-8). So decrease in Ferritin levels after N-163 is advantageous Liver, kidney and lung fibrosis
3. Total & LDL cholesterol decrease compared to AFO-202 Beta Glucan; Dysregulated cholesterol metabolism contributes to disease severity and cardiovascular risks (Ref: https://doi.org/10.1002/hep.26088). So decrease in total cholesterol and LDL levels after N-163 is advantageous. Liver and kidney disease
4. Galectin-3 decrease compared to AFO-202 Beta Glucan. Increased levels of galectin 3 have been associated with nonalcoholic steatohepatitis (NASH) and contribute to toxin-induced liver fibrosis (ref: https://doi.org/10.1053/j.gastro.2019.11.296). So decrease in Galaectin-3 levels after N-163 is advantageous.—Liver disease
5. HbA1C decrease and Gly.Alb decrease compared to AFO-202 Beta Glucan; But decrease in Gly. Alb more. GA/HbA1c ratio is significantly inversely associated with the presence and severity of NAFLD (ref: https://doi.org/10.1016/j.diabres.2016.12.01). So decrease in Gly. Alb after N-163 is advantageous Liver disease
6. Significant decrease in NEFA levels compared to AFO-202 Beta Glucan; NAFLD patients had significantly higher serum FFA levels than controls (ref: https://doi.org/10.1038/srep0583). So decrease in NEFA levels in advantageous —Liver, kidney and lung fibrosus disease
7. Significance decrease in IL8 and IL-6 compared to AFO-202 Beta Glucan IL-8 is strongly activated in CLD, thus likely contributing to hepatic inflammation. (ref: http://www.annclinlabsci.org/content/45/3/278.long). Decrease in IL6 and IL8 is therefore advantageous-Liver, kidney and lung fibrosus disease
8. Decrease in ballooning degeneration and liver fibrosis-NASH/Liver fibrosis

### Discussion

NASH or NAFLD is a serious chronic liver disease that at first is a metabolic imbalance leading to accumulation of fat in the liver, and the inflammatory response to excess fat accumulation leads gradually to fibrosis, jeopardizing the liver function, and beyond this, if the chronic inflammation continues, it may lead to cirrhosis and hepatocellular carcinoma [A8]. Increased plasma glucose and lipid levels contribute to direct lipid deposition in the liver apart from causing systemic inflammation which contribute to the development and worsening of NAFLD [A9]. A strategic approach to NAFLD therefore would be first to address the metabolic imbalance, which, based on our earlier findings, could be managed by administration of AFO-202 beta glucan [A5-A7], while the resolution of the already established fibrosis could be addressed by N-163 beta glucan, as shown in the present study. The present study has also proven that the combination of AFO-202 and N-163 is effective to address the chronic-inflammation-fibrosis cascade, preventing the culmination in cirrhosis or carcinoma.

In this study, the effects of AFO-202 and N-163 Beta 1,3-1,6 glucans were tested individually and in combination in STAM mice models of NASH. The decrease in body weight and liver weight was significantly lower only in the telmisartan group (FIG. 14).

The inflammation and ballooning scores were decreased mainly in the AFO-202 beta glucan groups, indicating that it helps in acting as an anti-inflammatory protective agent against NASH progression (FIG. 18). AFO-202 beta glucan has been shown to decrease inflammation-related cytokines in previous studies [A12]. This is further substantiated in the present study. However, fibrosis, which is the outcome of inflammation, was reduced mainly in the N-163 group, and the steatosis and NAFLD scores were decreased in the AFO-202+N-163 groups as effectively as in the telmisartan group (FIGS. 16A-B, 17A-B), indicating their application as an anti-fibrotic treatment agent in NASH. Beta glucans have been reported to help in alleviating obesity by acting on modulating transcription factor peroxisome proliferator-activated receptor (PPAR)-γ [A13]. This could be one probable mechanism against the anti-inflammatory and anti-fibrotic effects of AFO-202 and N-163 in the current study. Gut microbiota, which are dysregulated in metabolic syndrome, diabetes and dyslipidaemia, also lead to NASH by production of endotoxins. The prebiotic effects of the AFO-202 and N-163 beta glucans could also contribute to NASH alleviation by helping with gut microbiota's beneficial alteration [A1], which needs further validation.

AFO-202, N-163 beta glucans and their combination being essentially food supplements with established safety after decades of human consumption [A12], in contrast to a pharmacological agent such as telmisartan. The other beneficial effects of these beta glucans on obesity, diabetes and dyslipidemia [A5-A7, A13, A14] will help to address the associated NASH diseases, making them a wholesome preventive and therapeutic agent for NASH.

Furthermore, the mechanisms of the minute specific details though may be difficult, so additional evaluation of (i) gene expression for fibrotic and inflammatory markers in the STAM model after AFO-202, N-163 beta glucans administration could shed light on intricacies for a better understanding of these beta glucans in NASH, while (ii) evaluating common markers of tissue and organ fibrosis to other organ diseases such as PPAR-γ TGFb, TNFα, MCP-1, α-SMA, TIMP-1 [A15, A16] could add value to examining the possibilities of an extended application of these BRMGs in lung and kidney fibrosis as well. IL-6, having been already shown to be decreased by the AFO-202 beta glucan, which is a key cytokine implicated in inflammatory and fibrosis mechanisms [A17] of lung, liver and kidney [A18], is a specific biomarker worth evaluation in further studies.

Having been proven to be safe for human consumption as a food supplement, these two novel beta glucans, AFO-202 studied for 25 years and N-163, a larger multicentre study in NASH/NAFLD patients would be appropriate. However, one has to keep in mind the limitations of this study, including the fact that though this STAM model recapitulated human fatty liver disease to a great extent, there are still differences in the immunological mechanisms mediating inflammation between humans and mice, with human neutrophil-attracting chemokine IL-8 having no direct analogue in mice and differences in the corresponding immune cell subsets between mice and humans [A19].

### Conclusion:

This study was a comprehensive preclinical evaluation demonstrating the anti-fibrotic effects of N-163, anti-inflammatory effects of AFO-202 beta glucan and a combination of these two biological response modifier glucans in decreasing the NAS score in an established NASH model of fatty liver disease, STAM. Considering the safety of these two food supplements, a larger clinical study in NASH patients is recommended, and further research on these beta glucans and their beneficial effects through gene expression and common biomarkers of tissue and organ fibrosis is worthwhile, as the fundamental mechanisms of fibrosis in other organs such as the kidney and lungs have common mechanisms.

### Abstract

### Background

Non-alcoholic fatty liver disease (NAFLD) and non-alcoholic steatohepatitis (NASH) are highly prevalent conditions characterized by inflammation and fibrosis of the liver, progressing to cirrhosis and hepatocellular carcinoma if left untreated. Lifestyle disorders such as obesity, diabetes and dyslipidemia predispose one to and are associated with the disease progression. Conventional modalities are mainly symptomatic, with no definite solution. Beta glucan-based biological response modifiers are a potential strategy in lieu of their beneficial metabolic effects. Aureobasidium pullulans strains AFO-202 and N-163 beta glucans were evaluated for anti-fibrotic and anti-inflammatory potentials in a NASH animal model in this study.

### Methods:

In a STAM™ murine model of NASH, five groups were studied-(1) vehicle (RO water), (2) AFO-202 beta glucan; (3) N-163 beta glucan, (4) AFO-202+N-163 beta glucan, and (5) telmisartan (standard pharmacological intervention)-for eight weeks. Evaluation of biochemical parameters in plasma and hepatic histology including Sirius red staining and F4/80 immunostaining were performed.

### Results:

AFO-202 beta glucan significantly decreased inflammation-associated hepatic cell ballooning and steatosis. N-163 beta glucan decreased fibrosis and inflammation significantly (p value<0.05). The combination of AFO-202 with N-163 beta glucan significantly decreased the NAFLD Activity Score (NAS) compared with other groups.

### Conclusion:

This preclinical study proves the potential of AFO-202 and N-163 beta glucans as a potential preventive and therapeutic agent for NASH. After validation with additional markers of gene expression, extension of the benefits to ameliorate lung and kidney organ fibrosis may be considered.

Keywords: Non-alcoholic fatty liver disease (NAFLD); Non-alcoholic steatohepatitis (NASH); Beta glucans; Anti-fibrotic; Anti-inflammatory; Telmisartan

(Unique biological response modifier glucan N-163 yielding beneficial regulation of gut microbiota and fecal metabolome in an animal model; paving way for their effective utilization in human health and disease)

The gut microbiome and their metabolites reflect the nature of metabolism and the health of one's immune system, and are influenced by age and stress. Based on our earlier reports of clinical and pre-clinical studies in which AFO-202 and N-163 produced biological response modifier glucans either individually or in combination yield a beneficial outcome, we herein report the gut microbiota and fecal metabolome in a Stelic Animal Model (STAM) model which is one of the most metabolically stressed animal models.

### Methods:

In the STAM™ murine model, four groups were studied for eight weeks-(1) vehicle (RO water), (3) N-163 beta glucan, (4) AFO-202+N-163 beta glucan, and (5) telmisartan (standard pharmacological intervention). Fecal samples were collected at 6 weeks of age (before administration) and 9 weeks of age (before sacrifice). The gut microbiome was analysed using 16S rRNA sequence acquired by the next-generation sequencing. Fecal metabolome analysis was performed by gas chromatography-mass spectrometry (GC-MS).

### Results:

Gut microbial diversity increased greatly in AFO-202+N-163treated mice. Post-intervention, firmicutes decreased while Bacteroides increased which was highest in the AFO-202+N-163 group. Decrease in Turicibacter and Bilophila was highest in N-163 group. Increase in Lactobacillus was highest in the AFO-202+N-163 combination group. The Fecal metabolite Spermidine which is known to be beneficial against inflammation was greatly increased in N-163 group and increase in tryptophan was also observed in the N-163 treated group. Metabolites such as leucine, phenylalanine decreased and ornithine which are beneficial against chronic immune-metabolic-inflammatory pathologies such as cancer increased in the AFO-202+N-163 combination group.

### Conclusion:

In this study we show that treatment of mice with N-163 has anti-inflammatory effects relevant to organ fibrosis and neuroinflammatory conditions. When administered together, these beneficial effects may have anti-cancer activity. Collectively the results of this study suggest that long term studies of use of these agents which are natural food supplements are warranted.

With approximately 100 trillion micro-organisms existing in the human gastrointestinal tract, the microbiome is now considered as a virtual organ of the body. The microbiome encodes over three million genes producing thousands of metabolites compared to 23000 genes of the human genome and hence replaces many of the functions of the host influencing the host's fitness, phenotype, and health. Gut microbiota influences several aspects of human health including immune, metabolic and neurobehavioural traits [B1]. The gut microbiota ferments non-digestible substrates like dietary fibres and endogenous intestinal mucus which supports the growth of specialist microbes that produce short chain fatty acids (SCFAs) and gases. Major SCFAs produced are acetate, propionate, and butyrate. Butyrate is essential for the maintenance of colonic cells, helps in apoptosis of colon cancer cells, activation of intestinal gluconeogenesis, has beneficial effects on glucose and energy homeostasis and maintenance of oxygen balance in the gut and prevents gut microbiota dysbiosis.

Propionate is transported to the liver, where it regulates gluconeogenesis and acetate is an essential metabolite for the growth of other bacteria, as well as playing a role in central appetite regulation [B1]. The fecal metabolome represents the functional readout of the gut microbial activity and can be considered to be an intermediate phenotype mediating host-microbiome interaction. An average 67.7% (+18.8%) of the fecal metabolome's variance represents the gut microbial composition. Fecal metabolic profiling thus is a novel tool to explore links among microbiome composition, host phenotypes and disease states [B2]. Probiotics and pre-biotic nutritional supplements represent the major strategy other than fecal microbiota transplantation to restore the dysbiotic gut to a healthy state.

Beta glucans are one of the most promising nutritional supplements with established efficacy in metabolic diseases, diabetes, cancer, cardiovascular diseases and neurological diseases. Beta glucans produced from two strains AFO-202 and N-163 of a black yeast Aureobasidium pullulans derived beta glucan has been reported with beneficial effects in diabetes [B3], dyslipidemia [B4], ASD [B5, B6], Non-alcoholic steatohepatitis (NASH) [B7] and infectious diseases including COVID-19 [B8, B9]. The present study was undertaken as an extension of this NASH study to study the fecal microbiome and metabolome profile before and after administration of N-163 beta glucan individually and in combination with AFO-202.

### Methods:

### Mice

The study is reported in accordance with Animal Research: Reporting of In Vivo Experiments (ARRIVE) guidelines. C57BL/6J mice were obtained from Japan SLC, Inc. (Japan). All animals used in this study were cared for under the following guidelines: Act on Welfare and Management of Animals (Ministry of the Environment, Japan, Act No. 105 of Oct. 1, 1973), standards relating to the care and management of laboratory animals and relief of pain (Notice No.88 of the Ministry of the Environment, Japan, Apr. 28, 2006) and the guidelines for proper conduct of animal experiments (Science Council of Japan, Jun. 1, 2006). Protocol approvals were obtained from SMC Laboratories, Japan's IACUC (Study reference no: SP_SLMN128-2107-6_1). Mice were maintained in a specific pathogen-free (SPF) facility under controlled conditions of temperature (23±3° C.), humidity (50±20%), lighting (12-hour artificial light and dark cycles; light from 8:00 to 20:00) and air exchange.

The STAM model of NASH was produced as previously described [B7]. Mice were given a single subcutaneous injection of 200 μg streptozotocin (STZ, Sigma-Aldrich, USA) solution 2 days after birth and fed with a high-fat diet (HFD, 57 kcal % fat, Cat #HFD32, CLEA Japan, Inc., Japan) from 4-9 weeks of age. All mice develop liver steatosis and diabetes and at 3 weeks mice had established steatohepatitis, histologically.

Study groups: There were four study groups (n=8 mice per group) as described below.

Group 1: Vehicle: NASH mice were orally administered vehicle [RO water] in a volume of 5 mL/kg once daily from 6 to 9 weeks of age.

Group 2: N-163 Beta Glucan: NASH mice were orally administered vehicle supplemented with N-163 Beta Glucan at a dose of 1 mg/kg in a volume of 5 mL/kg once daily from 6 to 9 weeks of age.

Group 3: AFO-202 Beta Glucan+N-163 Beta Glucan: Eight NASH mice were orally administered vehicle supplemented with AFO-202 Beta Glucan at a dose of 1 mg/kg in a volume of 5 ml/kg once daily and orally administered vehicle supplemented with N-163 Beta Glucan at a dose of 1 mg/kg in a volume of 5 mL/kg once daily from 6 to 9 weeks of age.

Group 4: Telmisartan: Eight NASH mice were orally administered vehicle supplemented with Telmisartan at a dose of 10 mg/kg once daily from 6 to 9 weeks of age.

TABLE 4
Study design and treatment schedule

| | No. | Test | Dose | Volume | | |
|---|---|---|---|---|---|---|
| Group | mice | substance | (mg/kg) | (mL/kg) | Regimen | Sacrifice |
| 1 | 8 | Vehicle | — | 5 | PO, QD, | 9 wks |
| | | | | | 6-9 wks | |
| 2 | 8 | N-163 | 1 | 5 | PO, QD, | 9 wks |
| | | BetaGlucan | | | 6-9 wks | |
| 3 | 8 | AFO-202 | 1 | 5 | PO, QD, | 9 wks |
| | | Beta Glucan | | | 6-9 wks | |
| | | N-163 | 1 | | | |
| | | Beta Glucan | | | | |
| 4 | 8 | Telmisartan | 10 | 5 | PO, QD, | 9 wks |
| | | | | | 6-9 wks | |

### Test Substances

AFO-202 Beta Glucan and N-163 Beta Glucan were provided by GN Corporation Co Ltd., Japan. Telmisartan (Micardis®) was purchased from Boehringer Ingelheim GmbH (Germany).

### Randomization

NASH model mice were randomized into 5 groups of 8 mice at 6 weeks of age based on their body weight the day before the start of treatment. The randomization was performed by body weight-stratified random sampling using Excel software. NASH model mice were stratified by their body weight to get SD and the difference in the mean weights among groups as small as possible.

### Animal Monitoring and Sacrifice

The viability, clinical signs (lethargy, twitching, labored breathing) and behavior were monitored daily. Body weight was recorded daily before the treatment. Mice were observed for significant clinical signs of toxicity, moribundity and mortality before administration and after administration. The animals were sacrificed at 9 weeks of age by exsanguination through direct cardiac puncture under isoflurane anesthesia (Pfizer Inc.).

### Collection of Fecal Pellets Samples:

Frequency: Fecal samples were collected at the 6 weeks of age (before administration) and 9 weeks of age (before sacrifice).

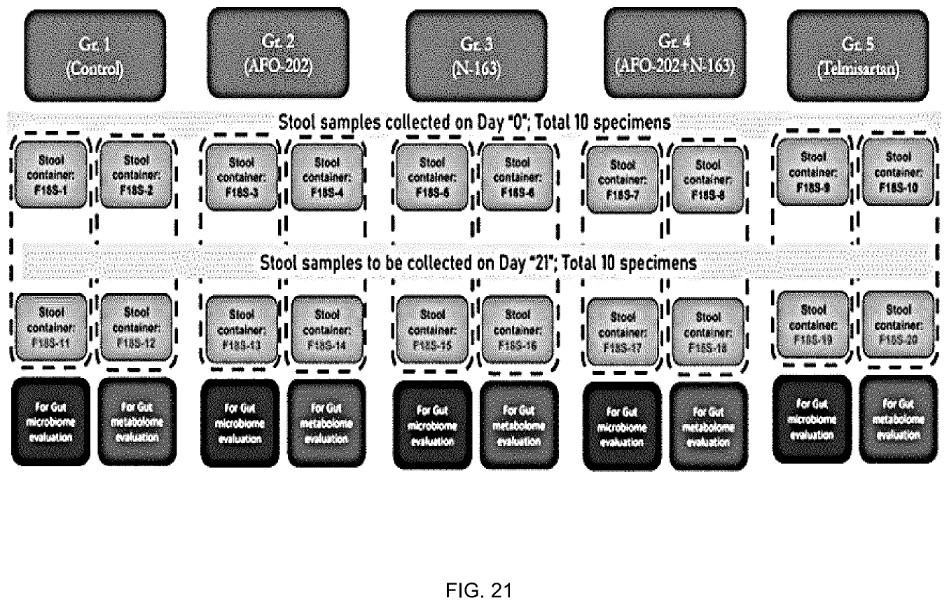

Procedure: At 6 weeks of age, fecal samples were collected from each mouse by clean catch method. Handle animals with clean gloves sterilized with 70% ethanol. A sterile petri dish was placed on the bench. Gently massage the abdomen, position the bottom of mouse over a fresh petri dish, and collect 1-2 fecal pellets. At sacrifice, fecal samples were aseptically collected from cecum. The tubes with feces were placed on ice immediately. These tubes were snap frozen in liquid nitrogen and stored at −80° C. for shipping. FIG. 21 shows the groups and the corresponding fecal sample number given for microbiome and metabolome analysis.

### Microbiome Analysis:

In this analysis, the 16S rRNA sequence data acquired by the next-generation sequencer from the fecal RNA was used to perform community analysis using the QIIME2 program for microbial community analysis. The raw read data in FASTQ format output from the next-generation sequencer was trimmed to remove adapter sequences and low QV regions that may be included in the data. Cutadapt was used to remove adapter sequences from DNA sequencing reads. Trimmomatic was used as read trimming tool for Illumina NGS data. The adapter sequence was trimmed using the adapter trimming program “cutadapt” if the Trimming of the region at the end of the read sequence overlapped the corresponding sequence by at least one base (mismatch tolerance: 20%). When reads containing N were present in at least one of Read1 and Read2, both Read1 and Read2 were removed.

### Illumina Adapter Sequence Information

Read 1 3′ end side
(SEQ ID NO: 1)
CTGTCTTCTATACACATCTCCGAGCCCACGAGAC
Read 2 3′ end side
(SEQ ID NO: 2)
CTGTCTTCTATACACATCTGACGCTGCCGACGA

Trimming of low QV regions was performed on the read data after processing using the QV trimming program “Trimmomatic” under the following conditions.

### QV Trimming Conditions

The window of 20 bases is slid from the 5′ side, and the area where the average QV is less than 20 is trimmed.

After trimming, only the reads with more than 50 bases remaining in both Read1 and Read2 were taken as output.

### Population Analysis

The microbial community analysis based on 16S rRNA sequence was performed on the sequence data trimmed in the previous section using the microbial community analysis program “QIIME2”. The annotation program “sklearn” included in QIIME2 is used to annotate the ASV (OTU) sequences.

Using “sklearn”, an annotation program included in QIIME2, the ASV (OTU) sequences obtained were annotated with taxonomy information [Kingdom (kingdom)/Phylum (phylum)/Class (class)/Order (order)/Family (family)/Genus/Species] based on the 16S rDNA database.

The data set of 16S rDNA database “greengenes” provided on the QIIME2 Resources site was used for the analysis. The ASVs (OTUs) obtained above were aggregated and graphed based on the taxonomy information a and the read counts of each specimen. Based on the composition of bacterial flora for each specimen compiled above, various index values for α-diversity were calculated.

### Metabolome Analysis:

After lyophilization of the fecal sample, about 10 mg of the sample was separated, extracted by the Bligh-Dyer method, and the resulting aqueous layer1 was collected and lyophilized. The residue was derivatized using 2-methoxyamine hydrochloride and MSTFA, and submitted to gas chromatography-mass spectrometry (GC-MS) as an analytical sample. 2-Isopropylmalic acid was used as an internal standard. In addition, an operational blank test was also conducted.

Analytical equipment used was GCMS-TQ8030 (Shimadzu Corporation); Column BPX-5 (film thickness 0.25 μm, length 30 m, inner diameter 0.25 mm, Shimadzu GC).

### Peak Detection and Analysis:

MS-DIAL ver.4.7 (http://prime.psc.riken.jp/compms/index.html) was used to analyze and prepare the peak list (peak height). In doing so, peaks that were detected in the QC samples and whose C.V. was less than 20% and whose intensity was more than twice that of the operating blank were treated as detected peaks.

Differential abundance analysis, PCA and PCA, OPLS-DA and Clustering analysis:

Principal component analysis (PCA) and orthogonal partial least squares-discriminant analysis (OPLS-DA) were performed to visualize the metabolic differences among the experimental groups. For principal component analysis, SIMCA-P+ver. 17 (Umetrics) was used. The normalized peak heights of the sample-derived peaks were used to perform principal component analysis using all samples and five points (F18S-12, F18S-14, F18S-16, F18S-18, and F18S-20). Transform was set to none, and Scaling was set to Pareto scaling. Differential metabolites were selected according to the statistically significant variable importance in the projection (VIP) values obtained from the OPLS-DA model. Hierarchical Cluster Analysis (HCA) and heat maps were performed using R (https://www.r-project.org/).

### Statistical Analysis

Statistical data were analysed using Microsoft Excel statistics package analysis software. Graphs were prepared using Origin Lab's Originb 2021 software. For normally distributed variables, t-test or ANOVA with Tukey HSD was used and P values <0.05 were considered significant. For OPLS-DA, values from two-tailed Student's t-tests were applied on the normalized peak areas; metabolites with VIP values >1 and P values <0.05 were included. The Euclidean distance and Ward's method were used to analyze the heat map. The mean and variance were normalized so that the mean is 0 and the variance is 1.

### Results:

There were no significant differences in mean body weights at any day during the treatment period between the control group and the other treatment groups. There were no significant differences in mean body weights on the day of sacrifice between the treatment groups.

### Gut Microbiome Analysis:

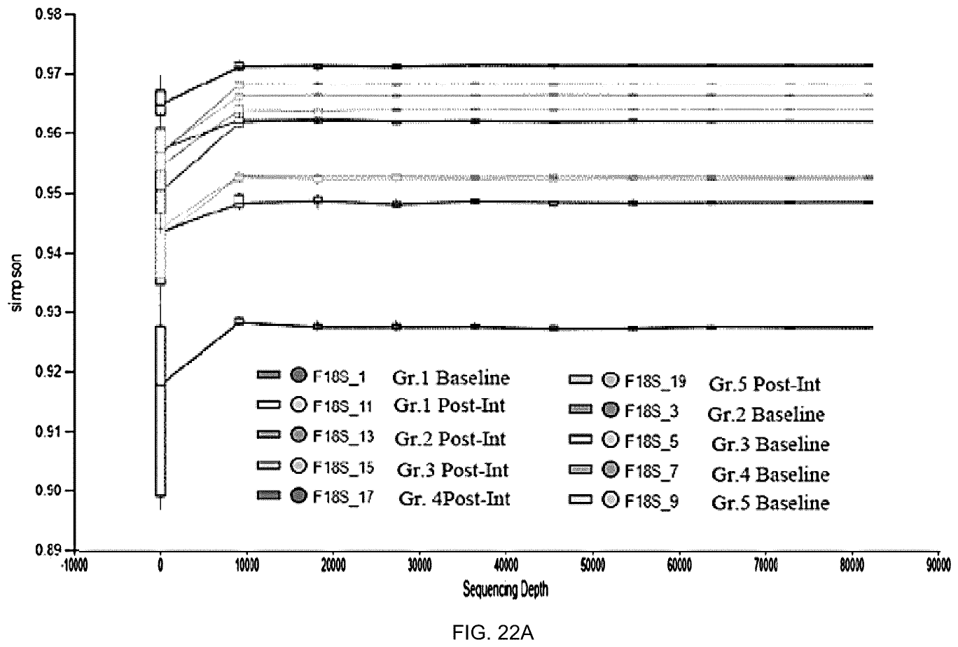

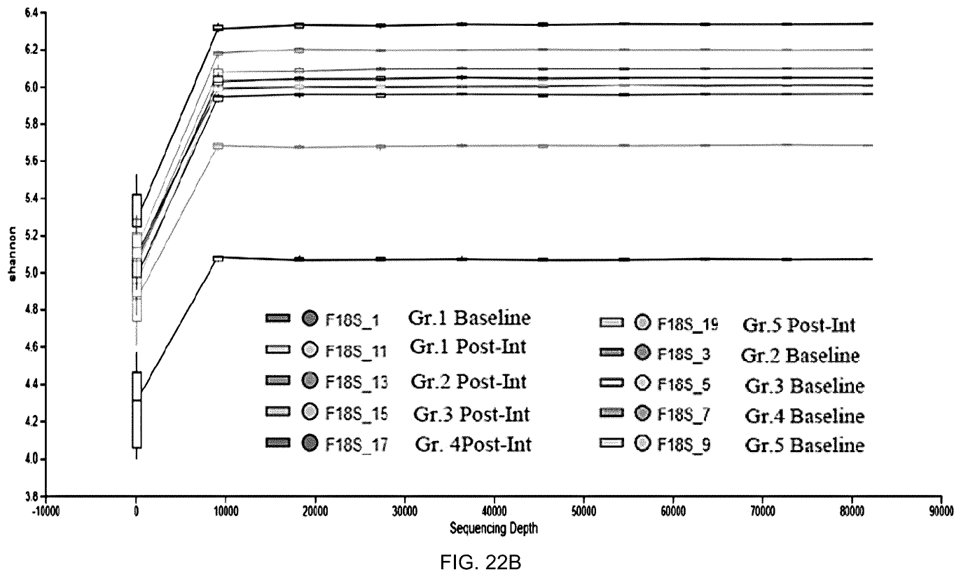

The alpha diversity indices (Simpson and Shannon)” s indices showed that the gut microbioal diversity was highest in the post-intervention AFO-202+N-163 (Gr.4) sample (FIGS. 22A and B)

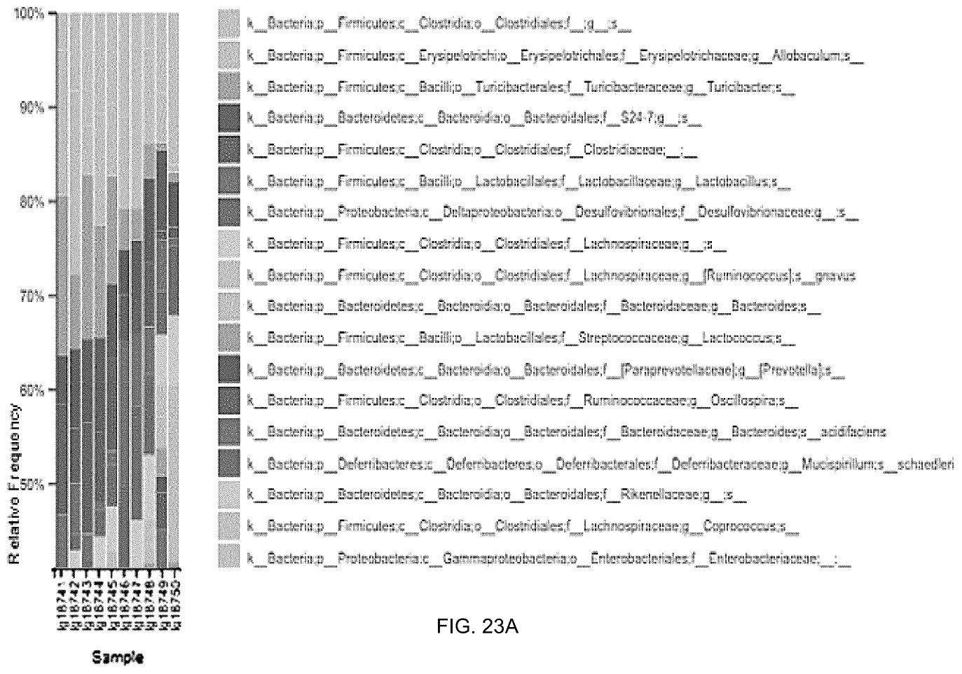

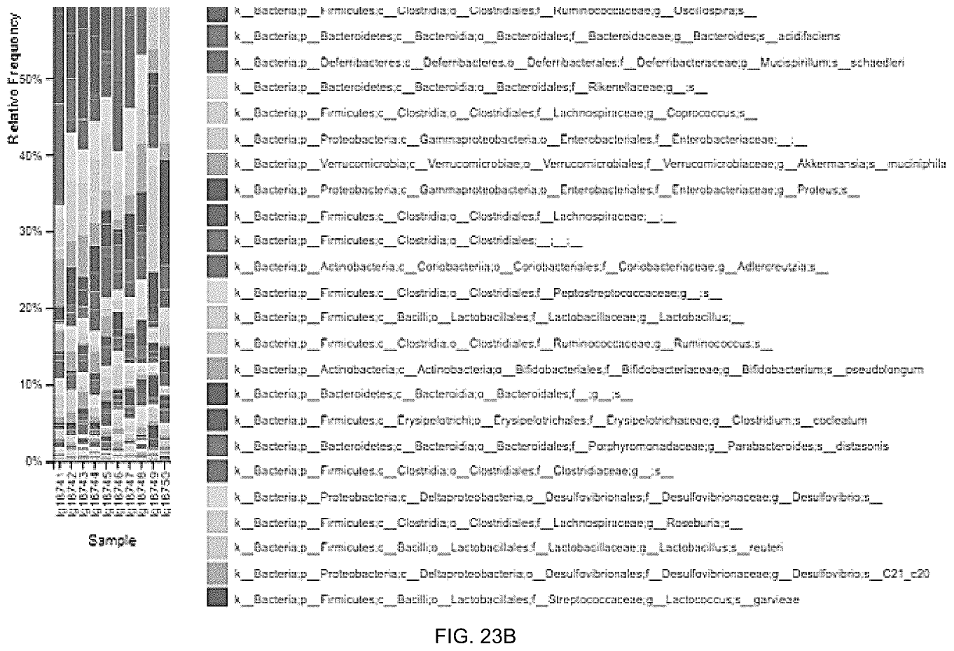

With regard to the taxonomic profiling, firmicutes represented the most abundant phyla followed by Bacteroidetes (FIG. 23)

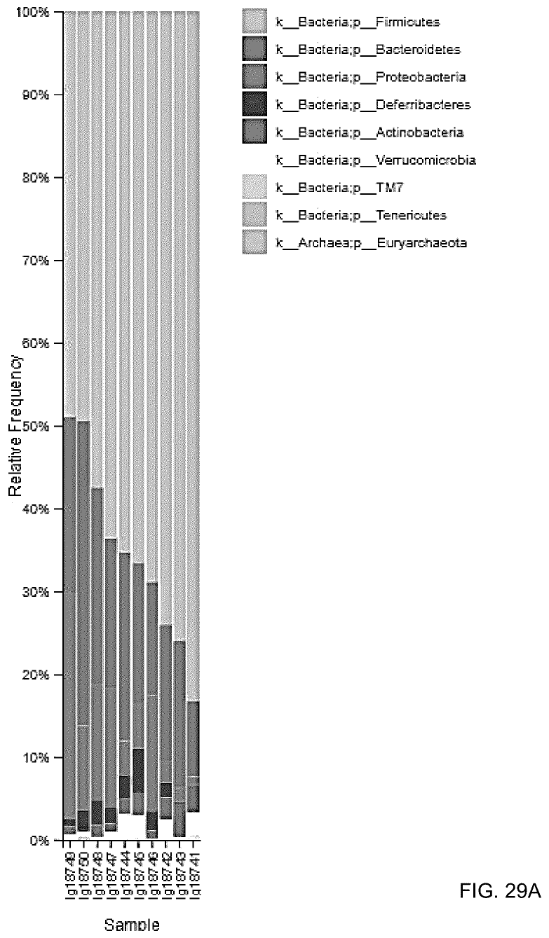

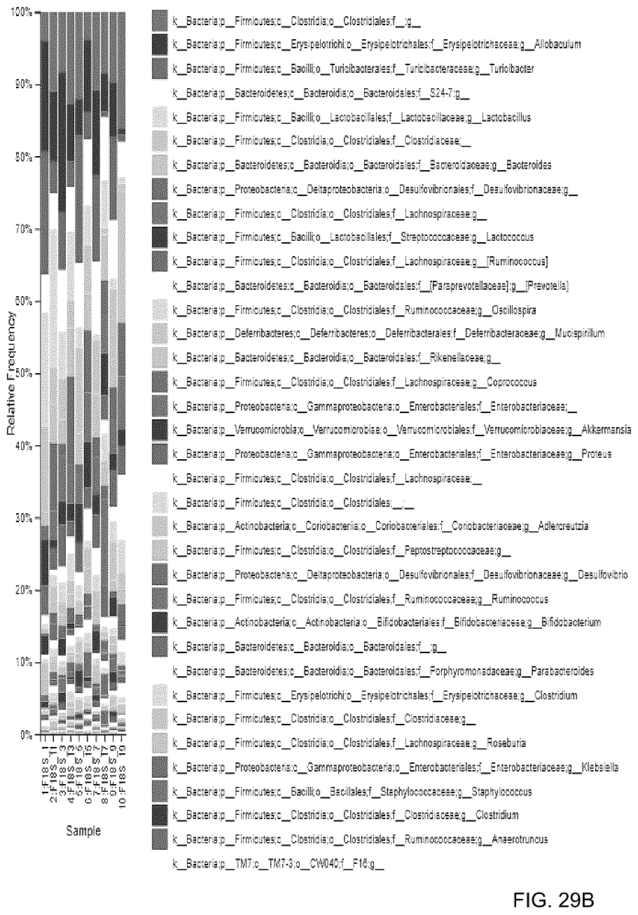

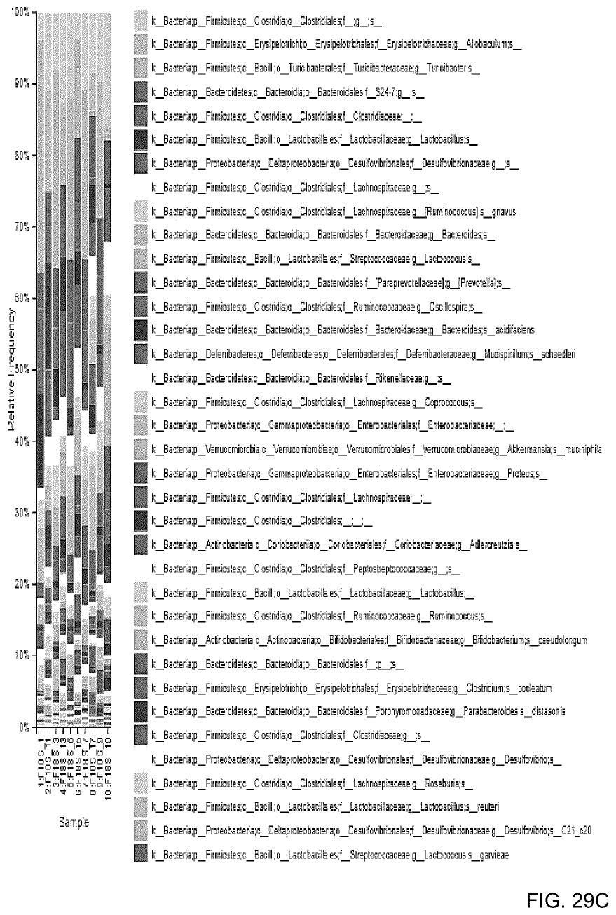

Post-intervention, firmicutes decreased while Bacteroides increased. This decrease in firmicutes and increase in Bacteroides was highest in the AFO-202+N-163 and Telmisartan compared to all other groups (FIG. 29).

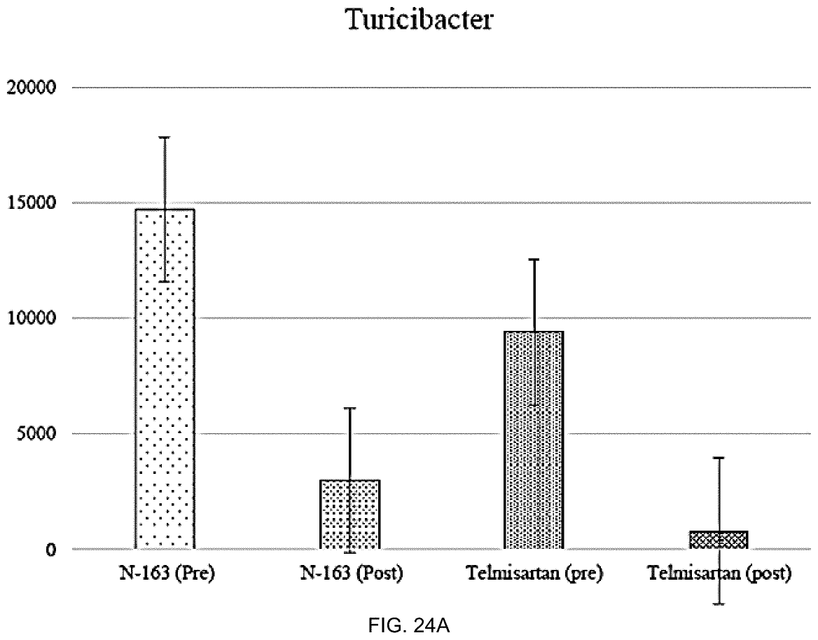

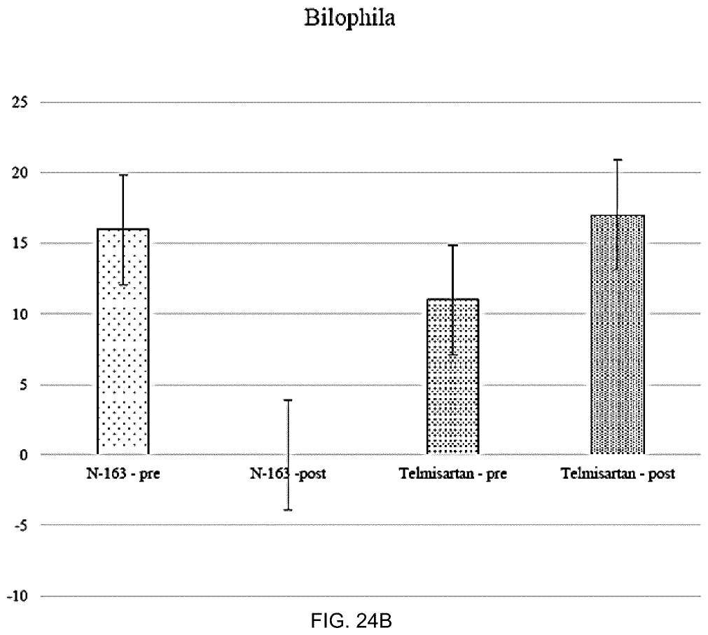

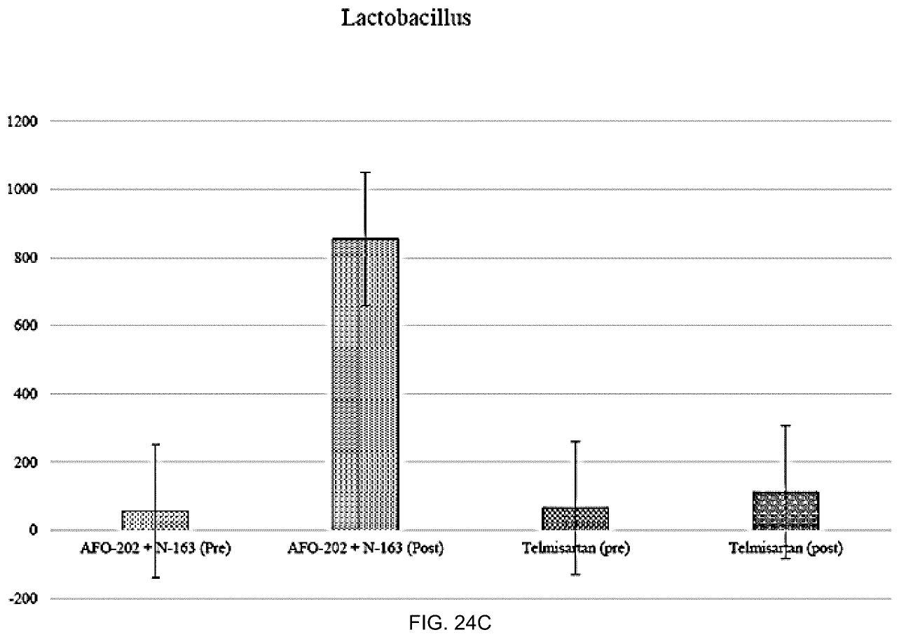

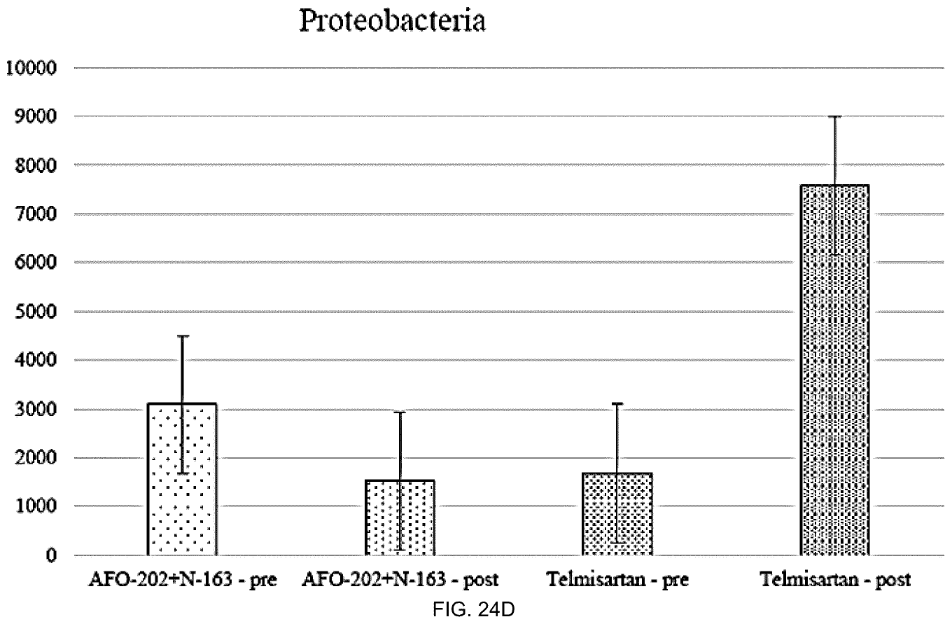

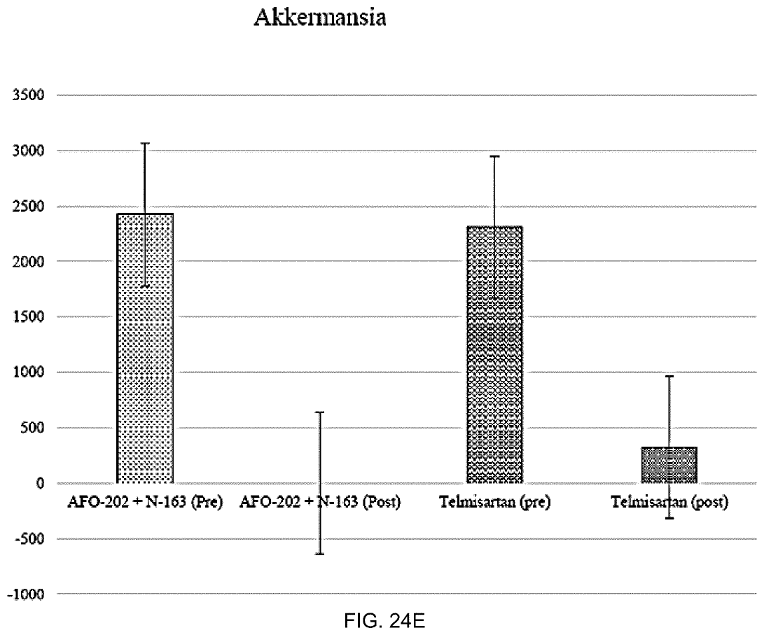

When individual taxa were analysed in each of the beta glucan groups comparing it to Telmisartan (standard), Turicibacter was highest in N-163 group. Bilophila increased in all the groups but decreased to 0 in N-163 group. Increase in Lactobacillus was highest in the AFO-202+N-163 combination group. Proteobacteria decreased in AFO-202+N-163 group while it increased in telmisartan. Decrease in Akkermansia was highest in AFO-202+N-163 group (FIG. 24).

### Fecal Metabolome Analysis:

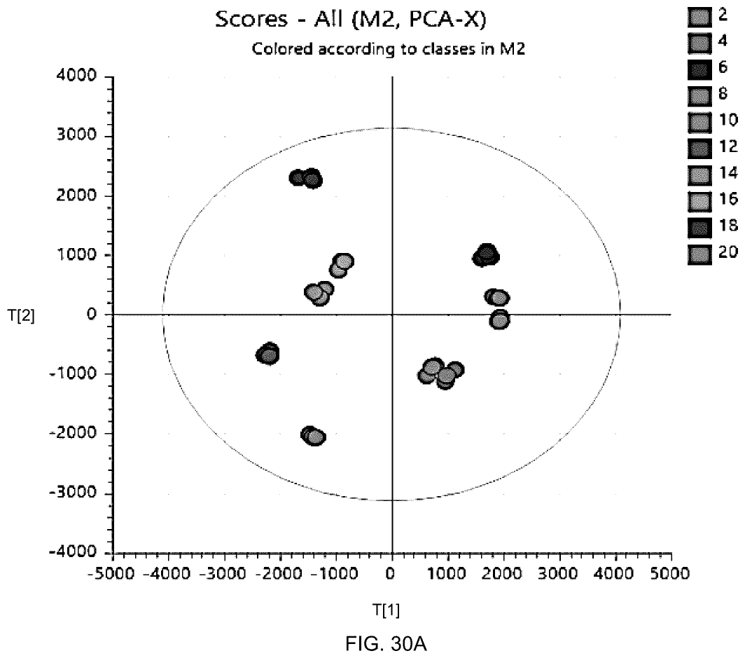

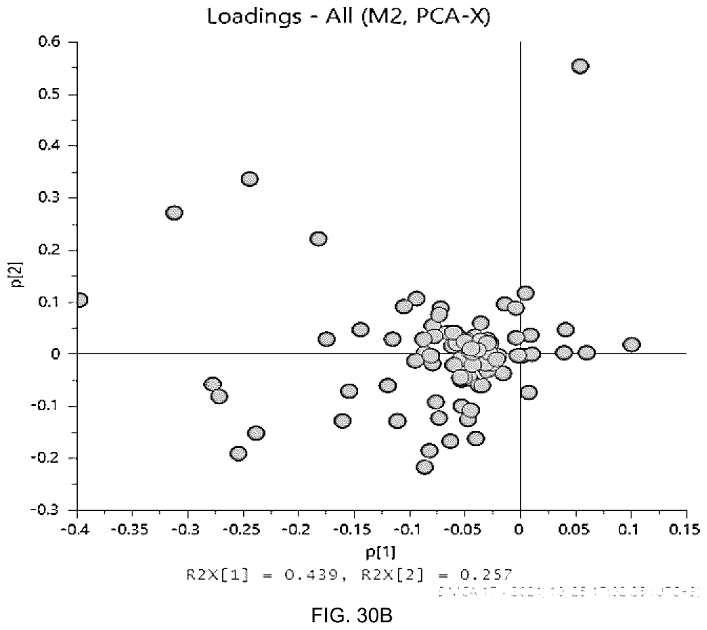

The resulting score plot of the principal component analysis using the normalized peak heights of the 10 samples (Pre- and post intervention of five groups), is shown in FIG. 30. The contribution of the first principal component was 55%, and that of the second principal component was 20%. Principal component analysis of five post-intervention samples (F18S-12, F18S-14, F18S-16, F18S-18, F18S-20) using the peak heights after normalization and the obtained score plot is shown in FIG. 30A and the loading plot in FIG. 30 B. The contribution of the first principal component was 49% and that of the second principal component was 34%.

The values which showed decrease post-intervention are highlighted in bold in the different groups. The number of peaks detected in the QC samples was 108, of which 53 peaks were qualitatively determined and 55 peaks were unknown.

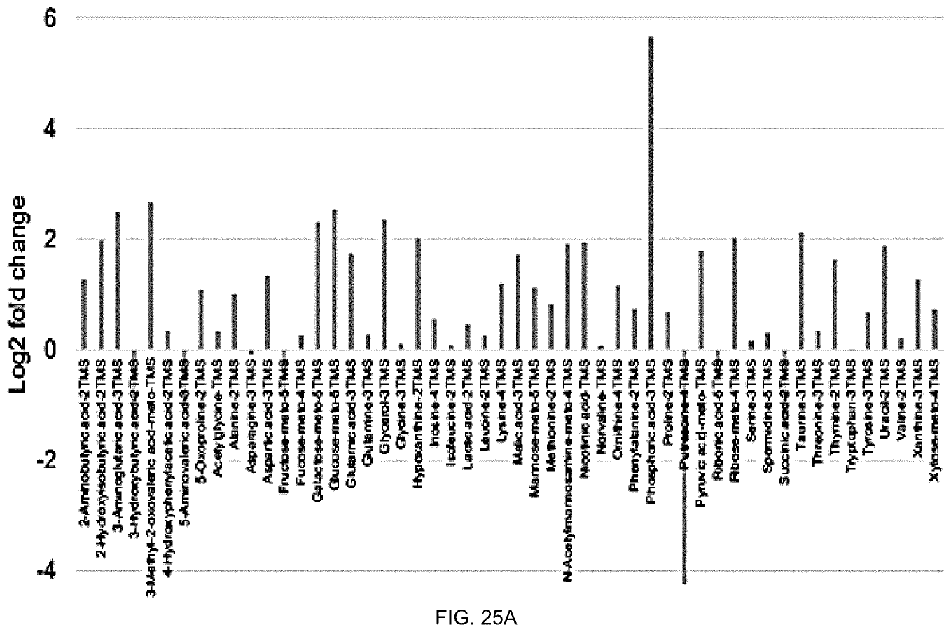

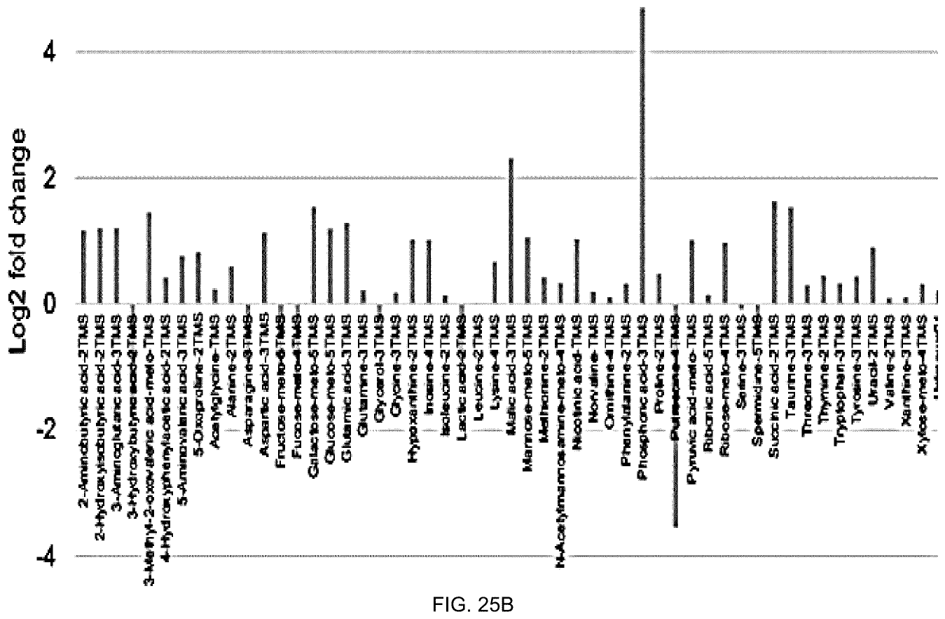

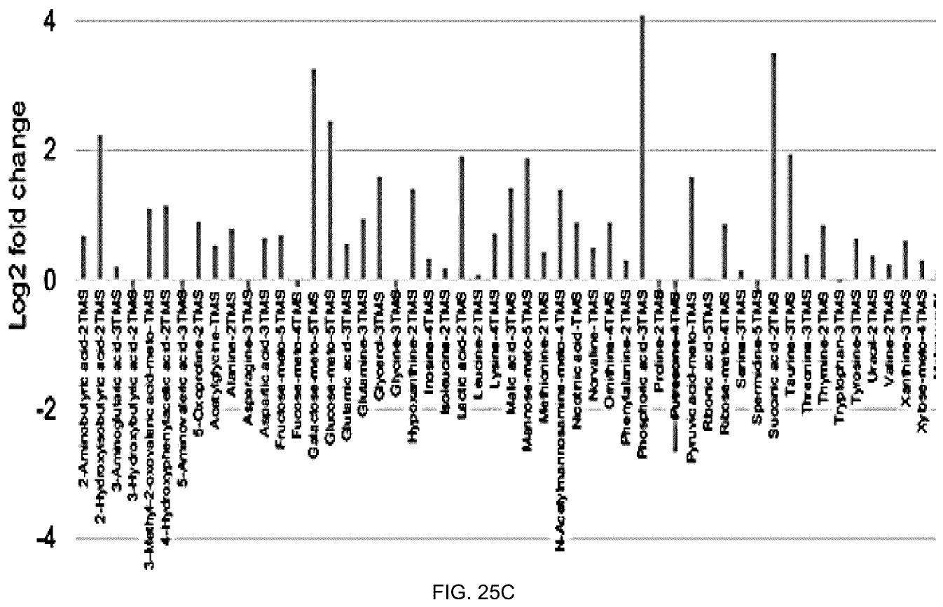

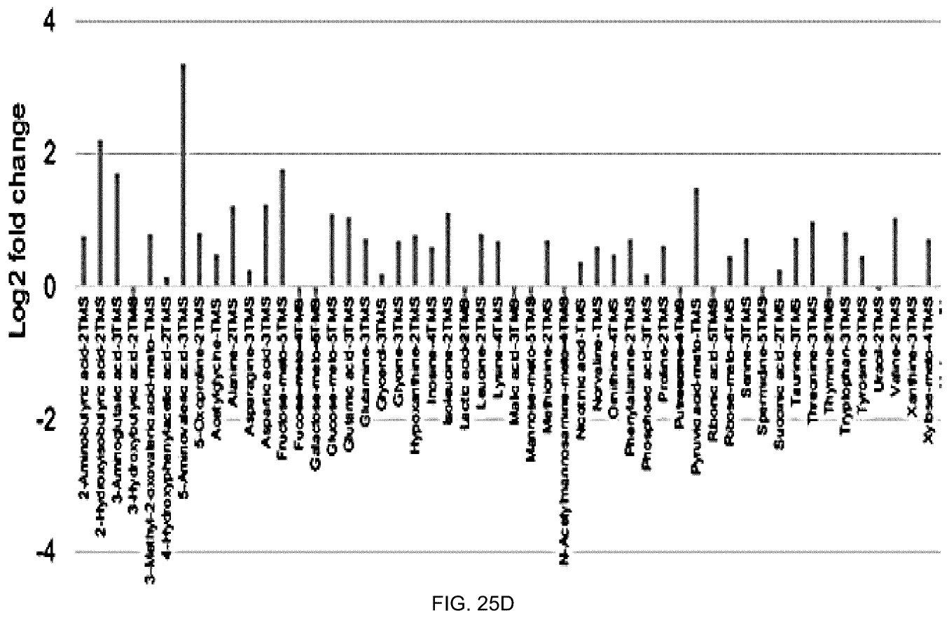

Differential abundance analysis, log 2 fold change results are shown in FIG. 25.

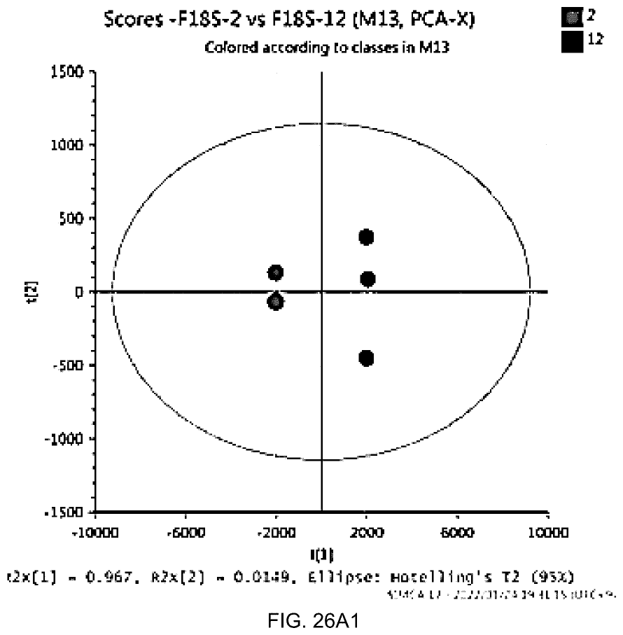

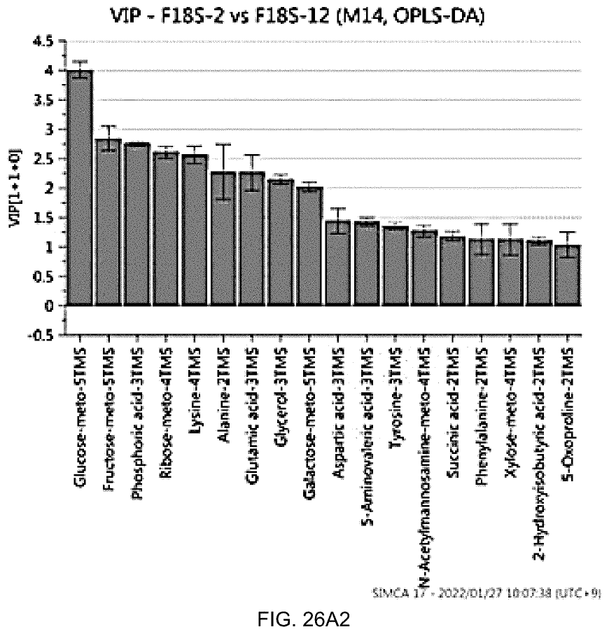

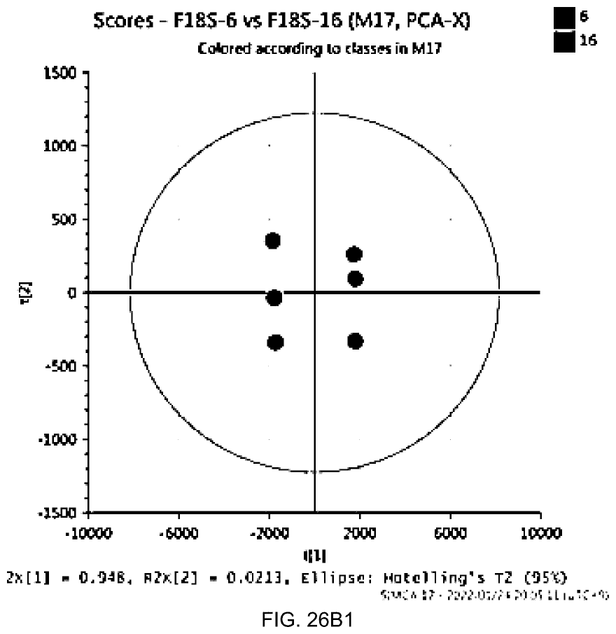

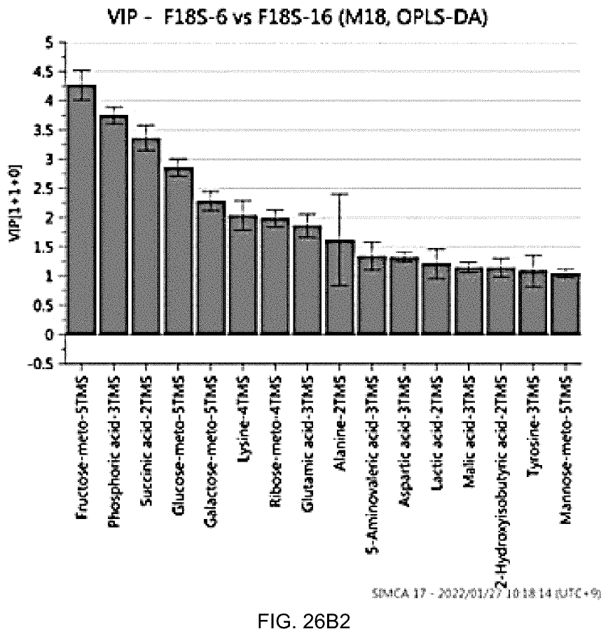

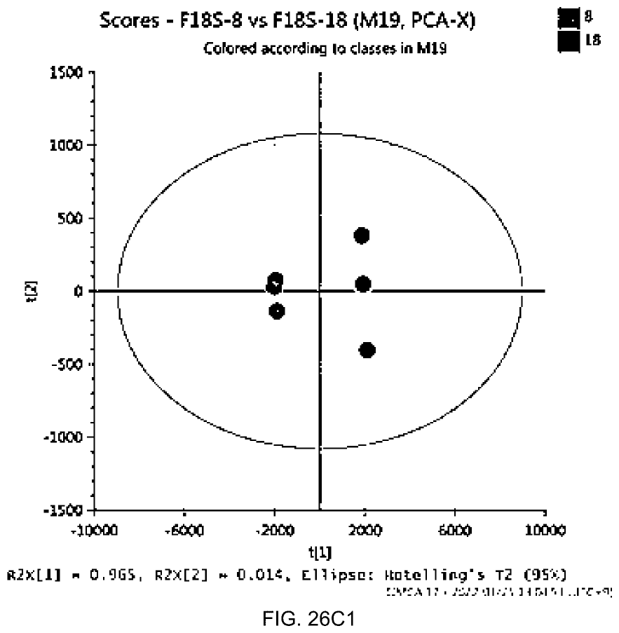

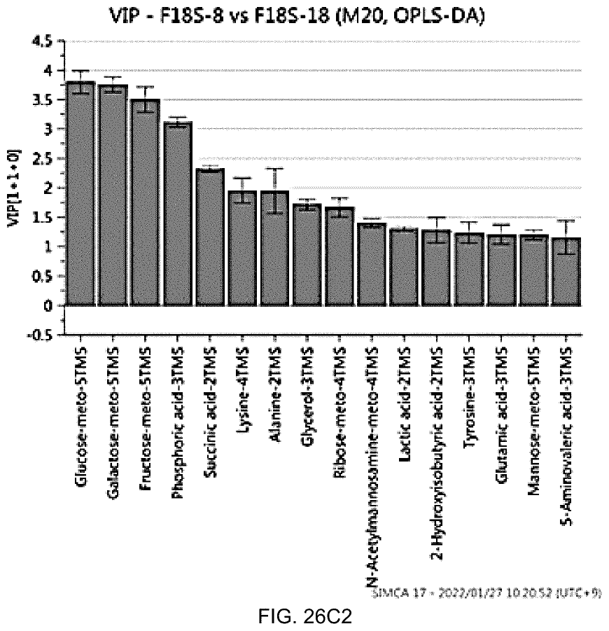

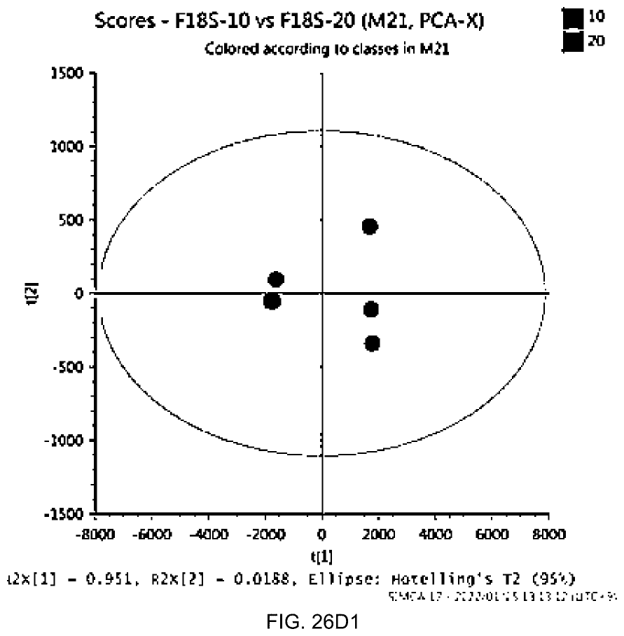

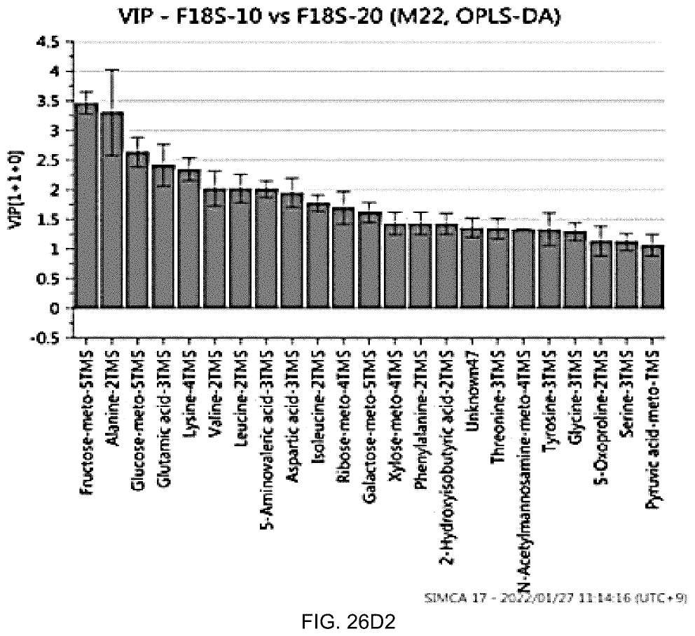

Score plots of PCA and compounds with a VIP value of 1 or higher in the OPLS-DA are shown in FIG. 26. The results of the principal component analysis of the control group showed that the contribution of the first principal component axis (PC1) was 96.7% and that of the second principal component axis (PC2) was 1.5%. In N-163 group, PC1 and PC2 contributed 94.8% and 2.1%, respectively. In AFO-202+N-163 group PC1 and PC2 contributed 96.5% and 1.4%, respectively. In the Telmisartan group, PC1 and PC2 contributed 95.1% and 1.9%, respectively.

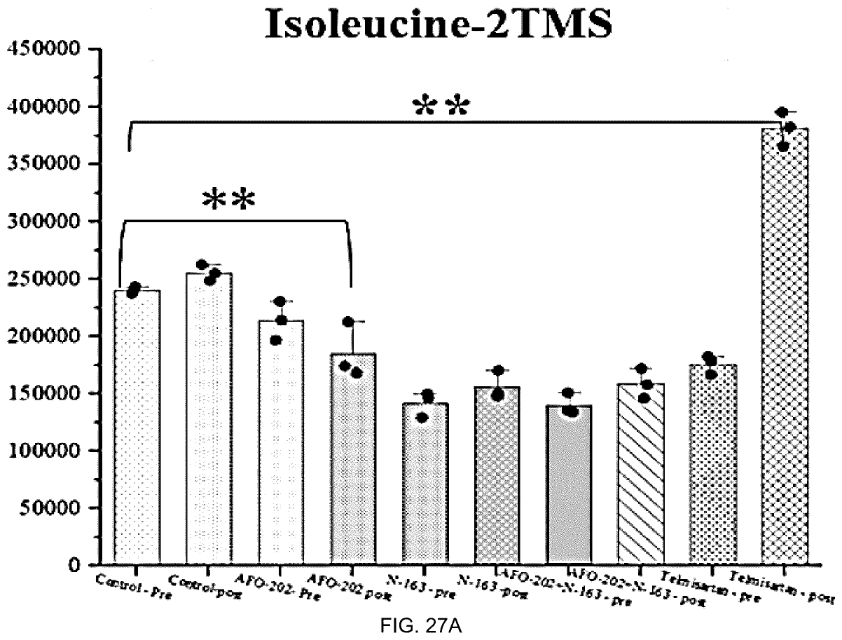

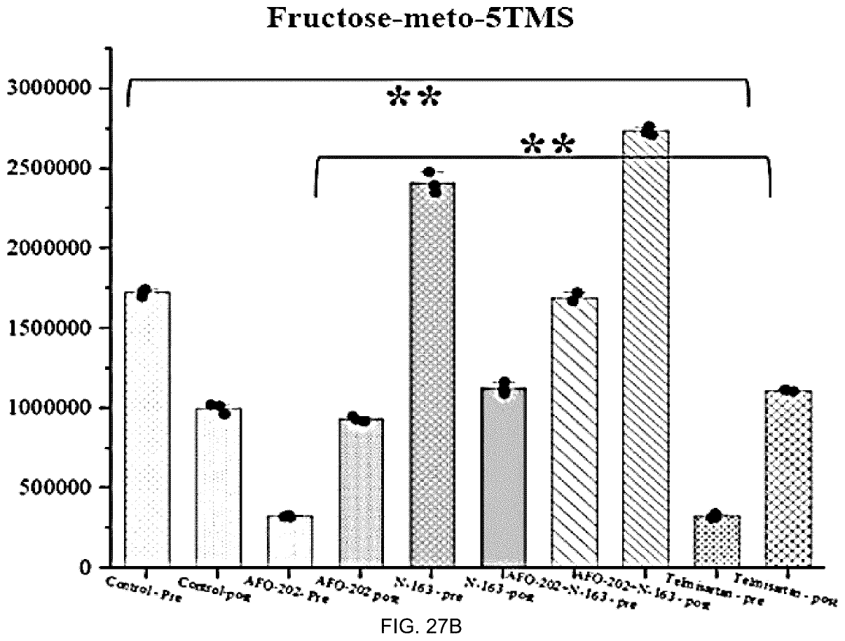

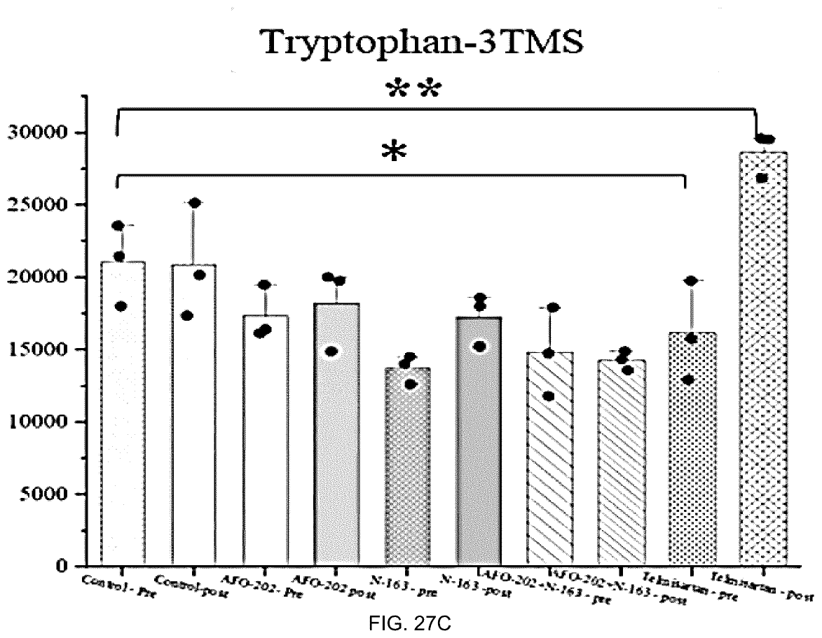

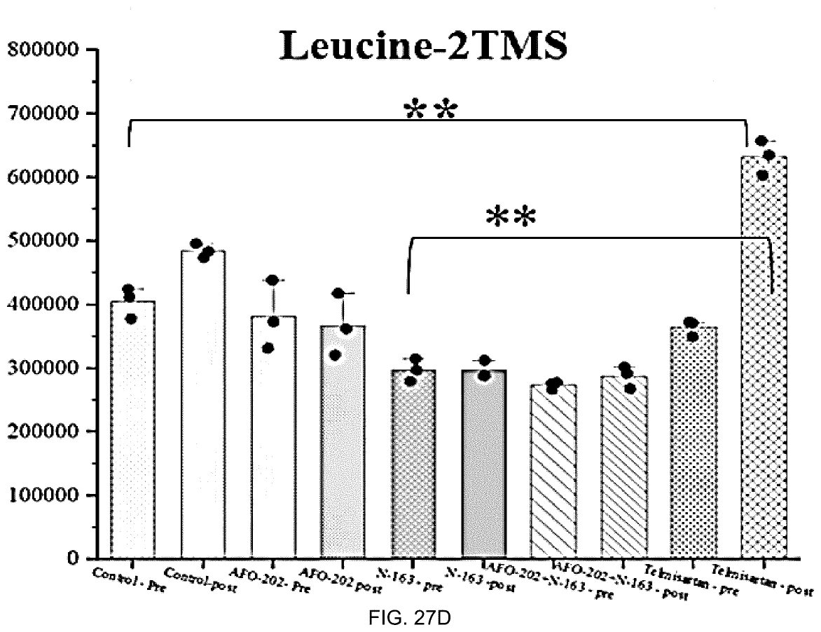

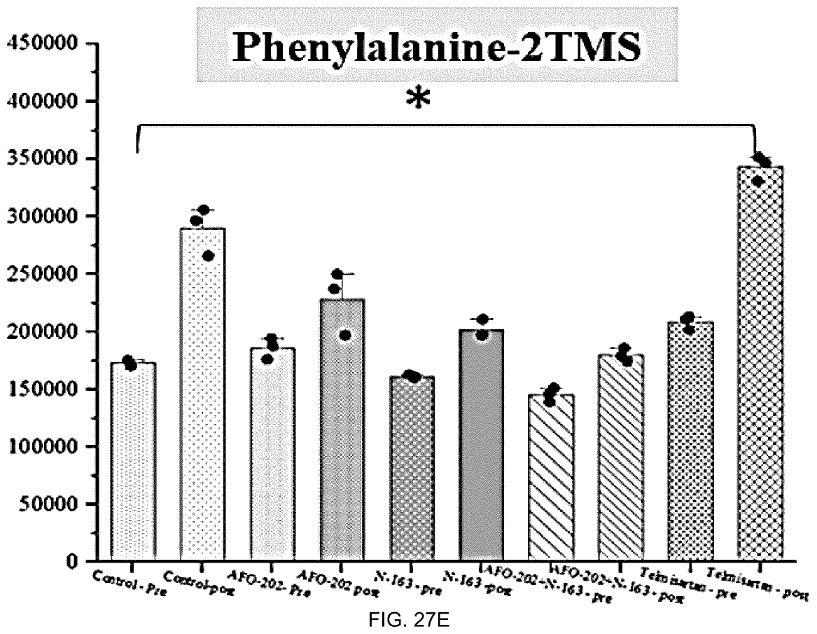

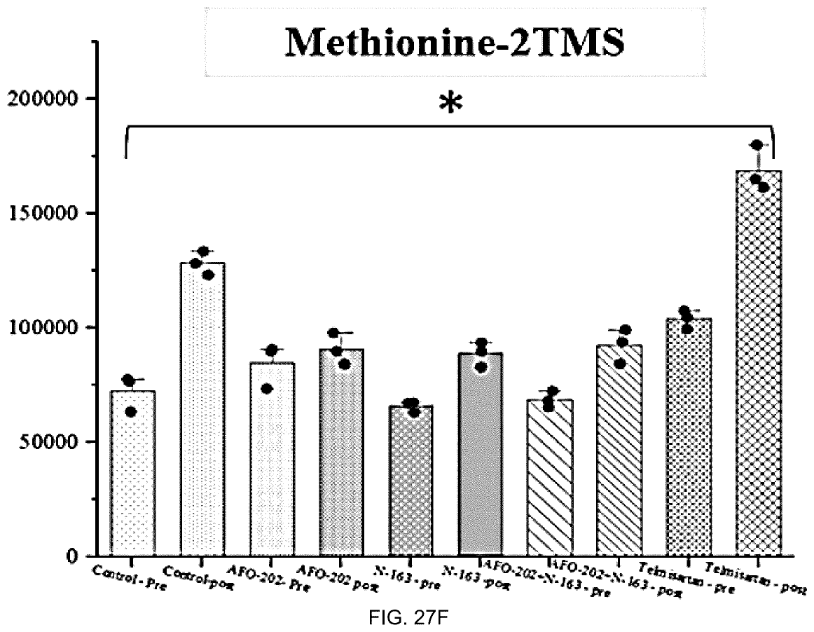

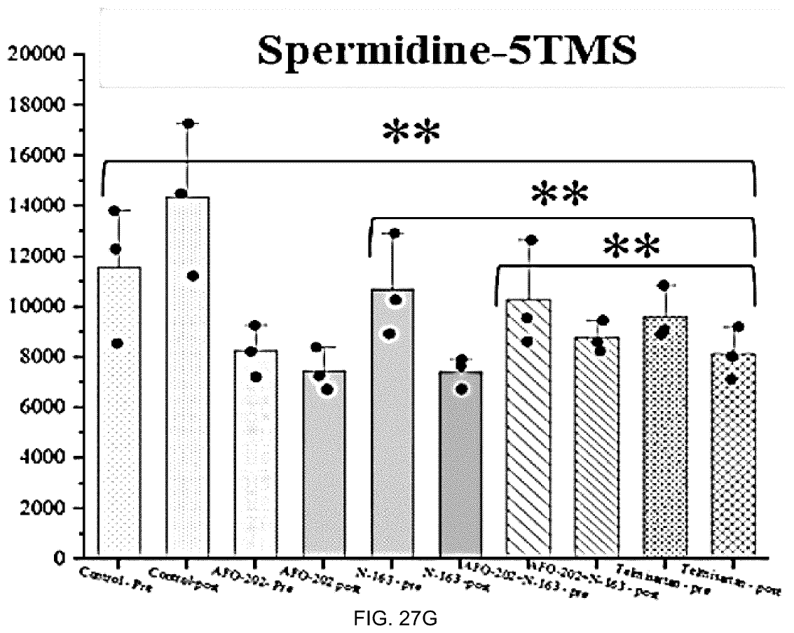

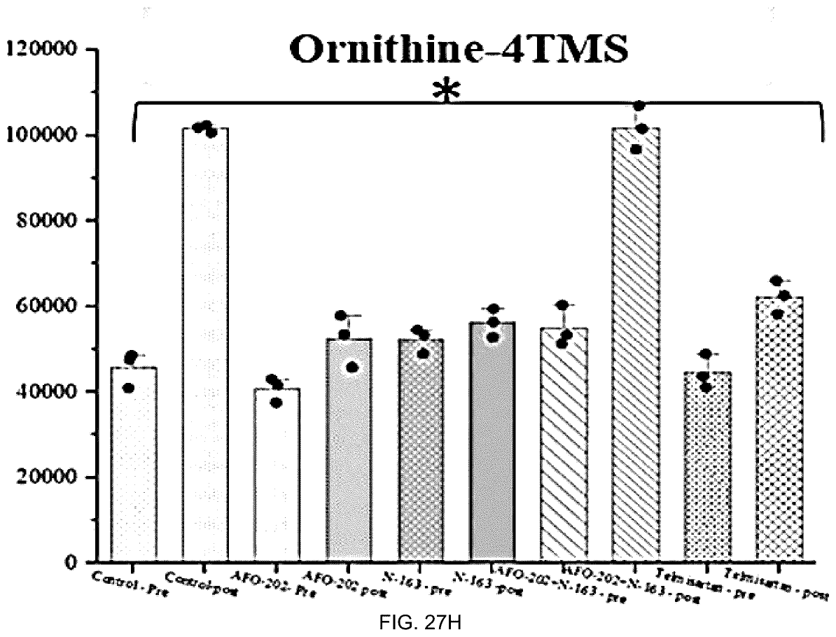

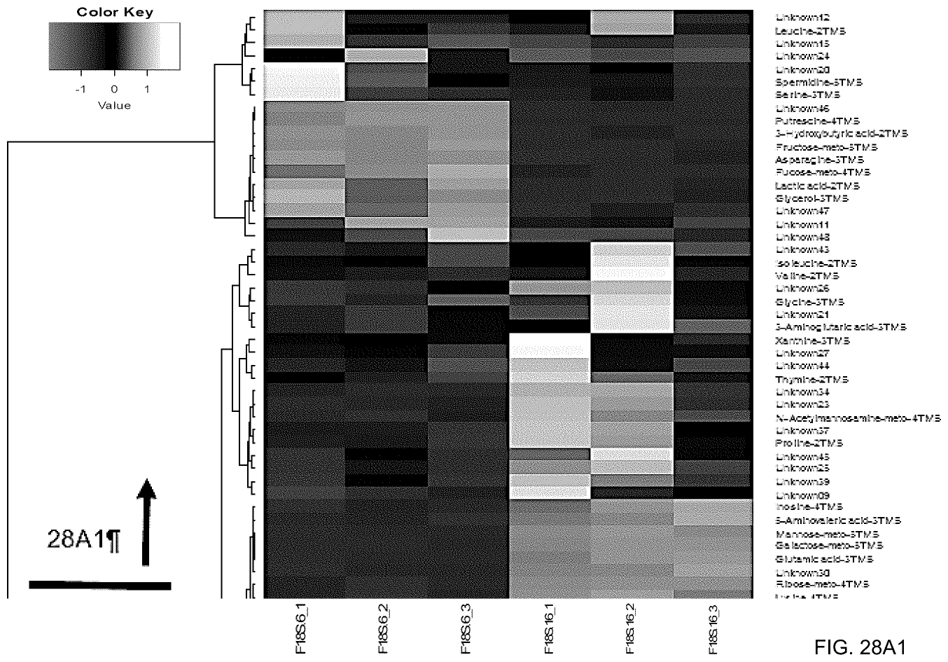

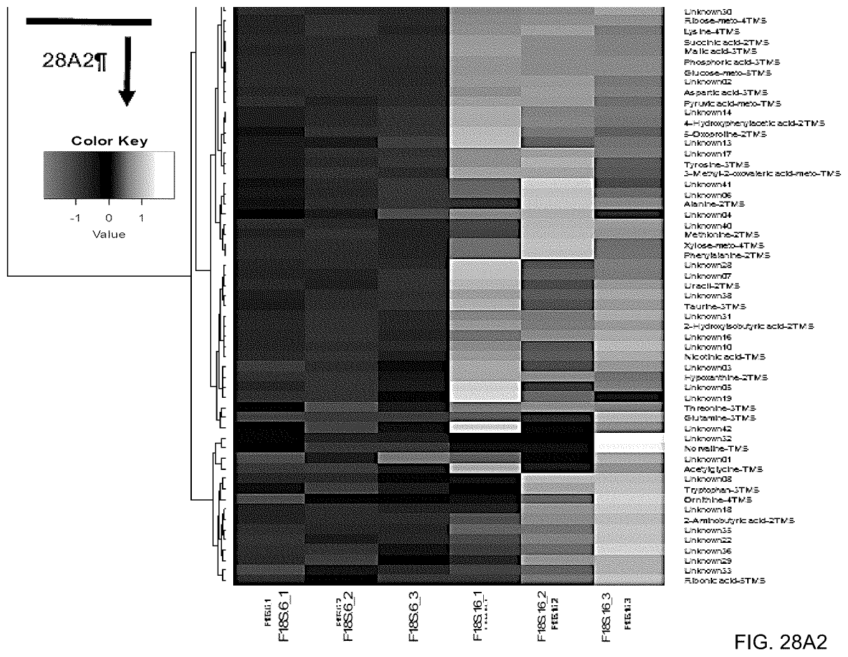

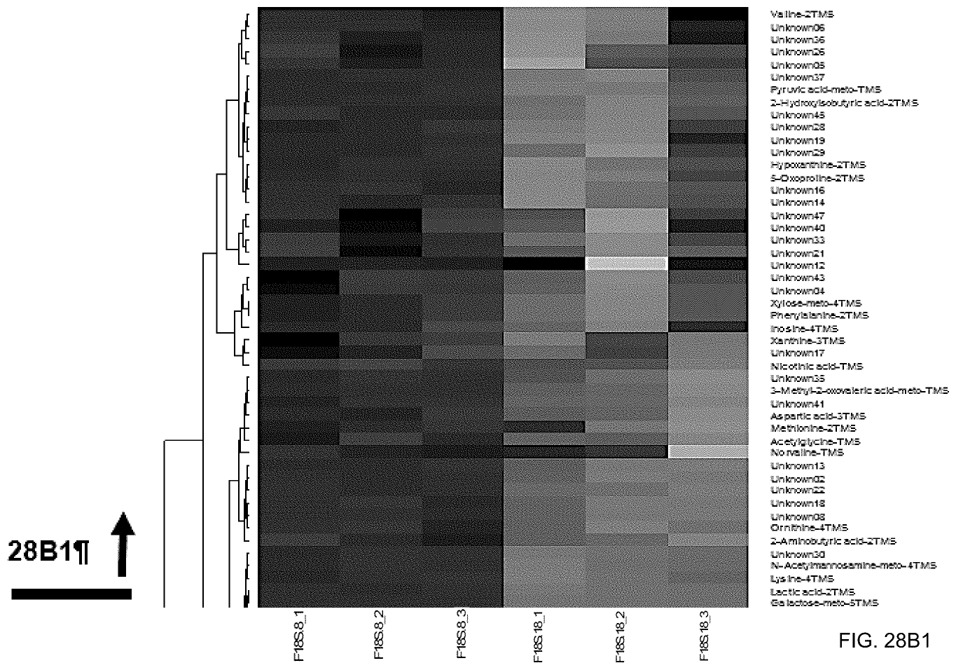

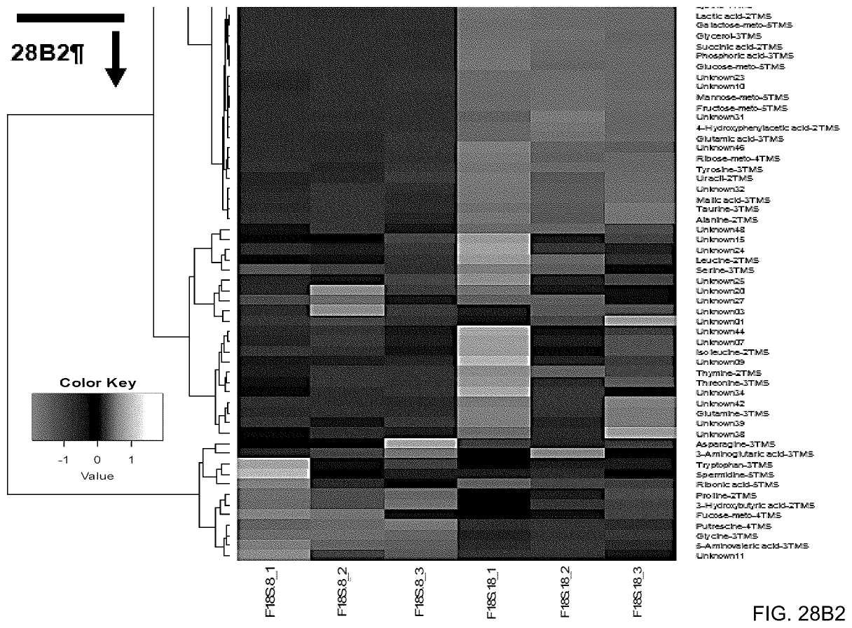

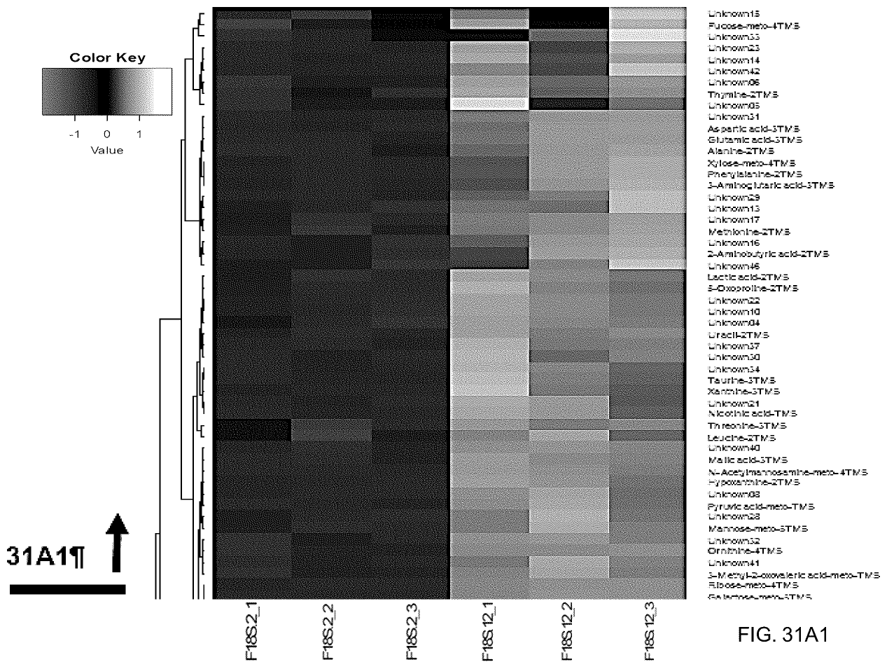

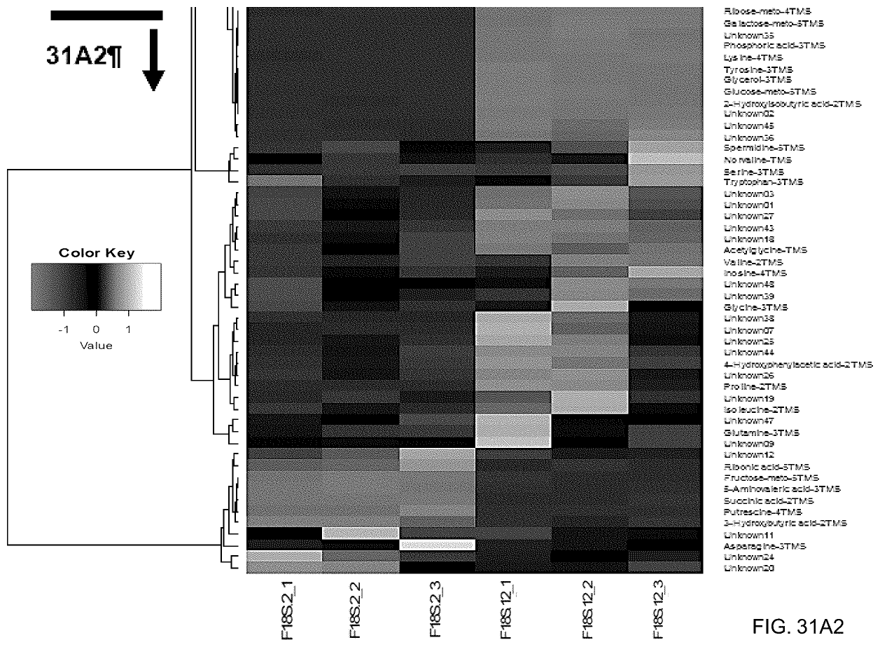

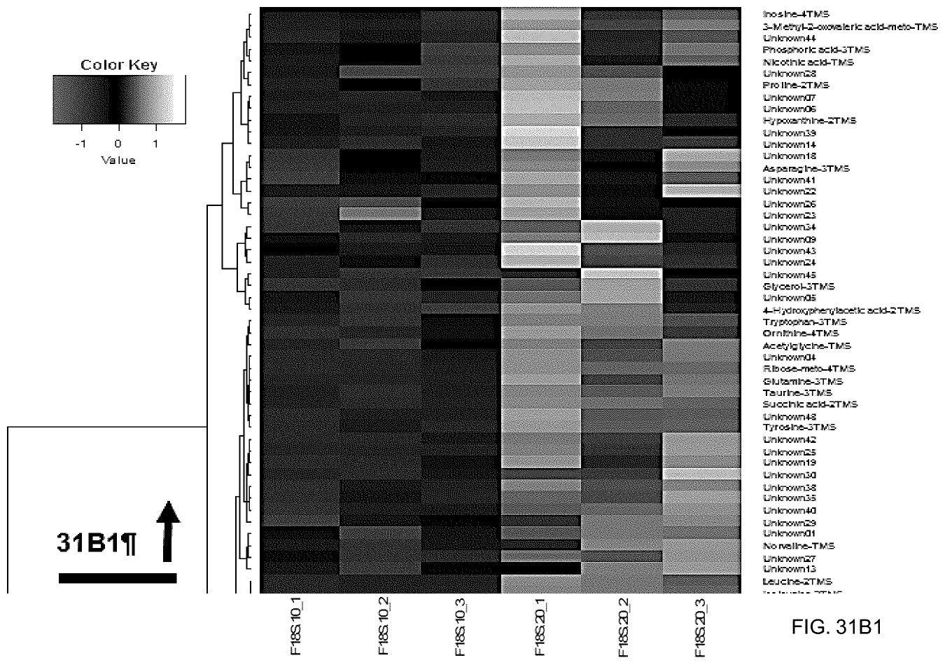

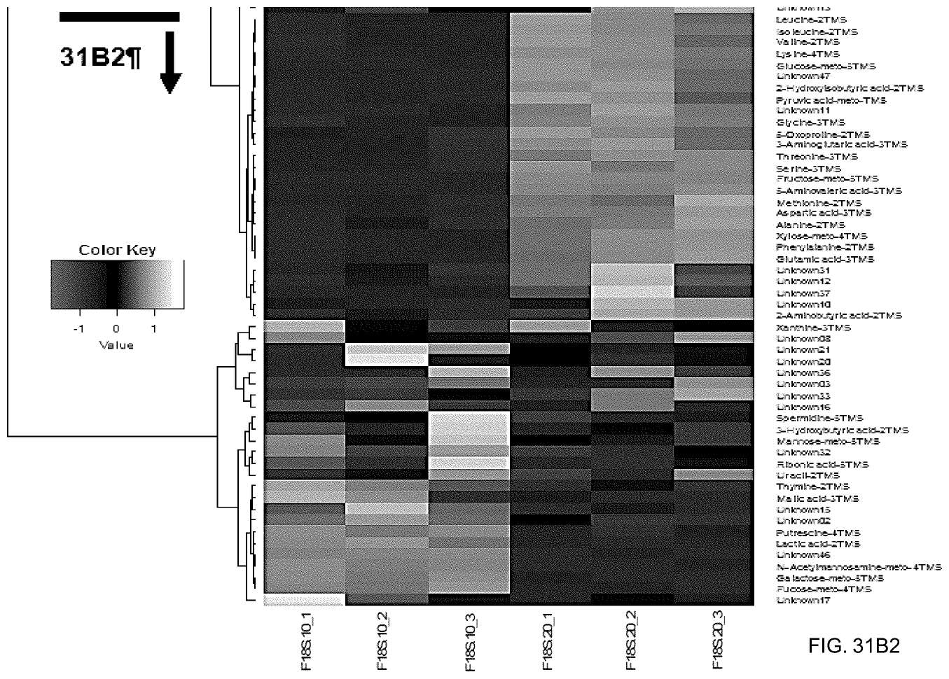

In all the groups except telmisartan treated mice, phosphoric acid showed the highest log 2 fold increase whereas putrescine showed the highest decrease. In regard to specific compounds, Increase in phosphoric acid was highest in N-163 followed by AFO-202+N-163 combination though not significant (p-value=0.21). Tryptophan decreased in AFO-202+N-163 combination group (not significant p-value=0.99) while in the other groups increased (FIG. 27). Decrease in Isoleucine (significant; p-value=0.004) and leucine was highest in N-163 group (significant; p-value=0.012) (FIG. 27). Decrease in phenylalanine was highest in AFO-202+N-163 combination group (not significant p-value=0.18) (FIG. 27). Methionine was found to be increased in all the groups (not significant p-value=0.14) (FIG. 27). Decrease in Spermidine was highest in N-163 group which was statistically significant (FIG. 27) (p-value=0.012). The increase in Ornithine was highest in AFO-202+N-163 combination group (FIG. 27J). Euclidean distance hierarchical clustering analysis demonstrating the different intensity levels of characteristic metabolites also matched the above observations (FIG. 28 and FIG. 31)

### Discussion

This is the first study to investigate the influence of beta glucans on the profiles of fecal gut microbiome and metabolome in a NASH model of mice. In this study we studied two different beta glucans produced by different strains of same species of black yeast A. pullulans. Beta glucans are obtained from different sources and the functionality depends on the source and extraction/purification processes [B11]. The beta glucans described in the study from AFO-202 and N-163 strains of the A. pullulans black yeast are unique as they are produced as an exopolysaccharide without the need for extraction/purification and hence the biological actions are superior [B12].

Further, both the beta glucans have the same chemical formula but different structural formula and hence exert diverse biological actions. The AFO-202 beta glucan has been reported to have superior metabolic benefits by regularization of blood glucose levels [B3] apart from immune enhancement in immune-infectious illnesses such as COVID-19 [B8, B9] and has been reported to produce positive effects on melatonin and alpha-synuclein neurotransmitters apart from improving sleep and behaviour in neurodevelopmental disorders such as ASD [B5, B6]. In the NASH animal study, AFO-202 beta glucan has been able to significantly decrease inflammation-associated hepatic cell ballooning and steatosis [B7]. The N-163 beta glucan has been able to produce immune-modulatory benefits in terms of regulating dyslipidaemia evident from balance of the levels of non-esterified fatty acids [B13] and decrease in fibrosis and inflammation in NASH [B7].

The combination of AFO-202 and N-163 beta glucans has been shown to decrease pro-inflammatory markers and increase in anti-inflammatory markers in healthy human volunteers [B14], decrease the NAFLD Activity Score (NAS) in the NASH model [B7] and significantly control immune-mediated dysregulated levels of IL-6, CRP and Ferritin in Covid-19 patients [B8, B9]. In the study done on gut microbiome analysis in ASD subjects, there was efficient control of enterobacteria apart from beneficial reconstitution of the gut microbiome favourable for producing benefits in ASD by AFO-202 beta glucan [B10]. In the current study, we sought to evaluate the benefits of AFO-202 and N-163 individually and in combination in the NASH animal model.

For these studies we used the Stelic Animal model (STAM) of NASH [B7, B15, B16]. In this model, mice are allowed to develop liver steatosis by injection of streptozotocin solution 2 days after birth and fed with a high-fat diet. This model recapitulates most of the features of the metabolic syndrome of NASH which occurs in humans wherein obesity and a high fat diet gives rise to diabetes, dyslipidaemia and liver steatosis. Therefore, the gut microbiome profiles and fecal metabolite profiles that are present at baseline can be considered to recapitulate that which is present in metabolic syndrome [B17, B18] which over time will produce pathophysiological problems in different organ systems of the body including the heart, liver, kidney apart from immune-metabolic interactions leading to a declined immune system with aging and its associated complications. Therefore, the present study will serve as a forerunner to study the effects of the beta glucans on different aspects of metabolic syndrome associated pathologies as well as conditions associated with immune-metabolic interactions including neurological disorders in which such immune-metabolic interactions have profound implications [B17].

### Relevance of Metabolome and Microbiome to NASH:

An abundance of bacterial species, such as Proteobacteria, Enterobacteria, and Escherichia coli has been reported in humans with non alcohol fatty liver disease (NAFLD). Greater abundance of Prevotella has been reported in obese children with NAFLD [B18, B19]. In the current study, a decrease in Enterobacteria with AFO-202 and a significant decrease in Prevotella with the combination of AFO-202+N-163 has been observed. In terms of fecal metabolites, an increase in Tryptophan was observed in N-163 but not greater than Telmisartan. In NAFLD, tryptophan metabolism has been found to be disturbed and supplementation of tryptophan has been found to be beneficial as it increased intestinal integrity and improved liver steatosis and function in a mouse model of NAFLD [B19]. Decreased production of butyrate has been shown to increase intestinal inflammation, increased gut permeability, endotoxemia and systemic inflammation. An increased abundance of 2-Hydroxyisobutyric acid has been observed in the AFO-202+N-163 group.

TABLE 5

| S. | N-163 | | N-163 + AFO-202 | | | |
|---|---|---|---|---|---|---|
| No | Increase | Decrease | Increase | Decrease | Application | Reference |
| 1 | | | | Tryptophan | increases intestinal | Chen J. Vitette L. Gut Microbiota Metabolites in NAFLD |
| | | | | | integrity and improves | Pathogenesis and Therapeutic Implications. Int J Mol Sci. |
| | | | | | liver steatosis and | 2020 Jul. 23 [symbol] 21(15): 5214 doi: 10.3390/ijms21155214 |
| | | | | | function | |
| 2 | Tryptophan | | | | Anti-cancer | Wu K K. Cytoguardin: A Tryptophan Metabolite against |
| | | | | | | Cancer Growth and Metastasis. Int J Mol Sci. 2021 |
| | | | | | | Apr. 26 [symbol] 22(9): 4490. doi: 10.3390/ijms22094490. PMID: |
| | | | | | | 33925793; PMCID: PMC8123408. |
| 3 | | Isoleucine | | | Helps against oxidative | Zhenyukh O. Gonzalez-A [symbol] M. Rodigues-Diez R R. |
| | | | | | stress, endothelial | Esteban V. Ruiz-Ortega M. Salacies M. Mas S. |
| | | | | | dysfunction and | Briones A M. Egido J. Branched-chain amino acids promote |
| | | | | | inflammation | endothelial dysfunction through increased reactive oxygen |
| | | | | | | species generation and inflammation. J Cell Mol Med. 2018 |
| 4 | | Leucine | | | | Oct. 22(10): 4948-962. doi: 10.1111/jcmm313759. Epub |
| | | | | | | 2018 Jul. 31 [symbol] PMID: 30063118: PMCID: PMC6156282. |
| | | | | | | Zhenyukh D. C [symbol] E. Ruiz-Ortega M. Sanchez M S. V [symbol] |
| 5 | | | | Phenylalanine | Helps against hepatic | Ta [symbol] K. Shimizu Y. Branched-chain amino acids |
| | | | | | encephalopathy | in liver diseases. World J Gastro [symbol] 2013 Nov. 21: |
| | | | | | | 19(43): 7620-9. doi: 10.3748/ [symbol] |
| 6 | | Spermidine | | | Anti-inflammatory | Choi Y H. Park H Y. Anti-inflammatory effects of spermidine |
| | | | | | | in lipopolysaccharide-stimulated BV2 microglial cells. J |
| | | | | | | Biomed Sci. 2012 Mar. 20: 19(1): 31. doi: 10.1186/1423- |
| | | | | | | 0127-19-31. PMID: 22433014; PMCID: PMC3320531. |
| 7 | | | Ornithine | | Anti-cancer | Le Ga [symbol] G. Gu [symbol] K. Kelling: ay L. Te [symbol] A [symbol] Ten |
| | | | | | | Hoppen R. Ke [symbol] ley E K. Se [symbol] G M. Ibrahim A. [symbol] |
| | | | | | | A. Metabolite quantification of faecal extracts from colorectal |
| | | | | | | cancer patients and healthy controls. Oncotarget. 2018 Sep. |
| | | | | | | 7: 9(70): 33278-33289. doi: 10.18632/oncotarget26022. |
| | | | | | | Erratum in: Oncotarget. 2019 Feb. 26: 26: 10( [symbol] ): 1660. |

[symbol]

indicates data missing or illegible when filed

Increase in tryptophan after administration of N-163 beta glucan has potential as an anti-cancer agent. Decrease in Amino-acids such as Iso-leucin and Leucine will help against oxidative stress, endothelial dysfunction and inflammation. Increase in spermidine helps to alleviate inflammation. Decrease in tryptophan after administration of combination of AFO-202 and N-163 beta glucans is beneficial as it increases the intestinal integrity and improves liver steatosis and function. Decrease in phenylalanine helps against hepatic encephalopathy that occurs during liver failure. Increase in Ornithine in combination of AFO-202 and N-163 has potential as an anti-cancer agent

### Other Implications:

In overweight/obese humans, low fecal bacterial diversity was reported to be associated with more marked increase in fat tissue dyslipidemia, impaired glucose homeostasis and higher low-grade inflammation [B26]. In the current study, the bacterial diversity increased after intervention, especially, the combination (AFO-202+N-163) group showed the highest diversity in the Shannon and Simpson indices (FIG. 23). Most studies report an increase in Firmicutes and decrease in Bacteroidetes to be directly proportional to the gain in body weight [B26, B27]. In the present study, there is a clear decrease in Firmicutes and increase in Bacteroidetes in all the groups post-intervention but the highest was in the combination (AFO-202+N-163) and Telmisartan groups (FIG. 23,24 Spermidine is a metabolite which has been associated with inflammation and cancer [B28]. Decrease in Spermidine was highest in N-163 group (FIG. 29). Ornithine has been found to be decreased in colorectal cancer patients [B29]. Increase in Ornithine was highest in the combination (AFO-202+N-163) in the present study. Lactobacillus is a common probiotic which is used for prophylaxis against and treatment of chronic conditions such as cancer [B31] as well as promoting better health. Increase in lactobacillus was highest in the combination (AFO-202+N-163) (FIG. 25). Steroids are common immunosuppressants which are used to treat chronic auto-immune conditions as well as organ transplant recipients. Use of Steroids has been reported to cause increase in E-Coli, enterococcus while decrease in Bacteroides [B31]. In the current study, the control of enterobacteria with increase in Bacteroides by AFO-202, N-163 as well as their combination make them worthy adjuncts for medications such as steroids as well. The beneficial effects on gut microbiota and alleviating gut dysbiosis is more profound in the combination of AFO-202+N-163 compared to N-163 alone. Especially increase in lactobacillus which is one of the most common bacteria used in probiotics having been increased in the combination of AFO-202+N-163 makes it more significant.

### Conclusion:

In summary, black yeast A. pullulans' two strains AFO-202 and N-163 produced beta glucans, increase the gut microbial diversity, control harmful bacteria, promote healthy ones apart from producing beneficial differences in fecal metabolites, all indicative of a healthy profile both individually and in combination in this NASH animal model. The combination of AFO-202 and N-163 might help to preserve overall health, serving as a preventive agent against chronic inflammatory and immune-dysregulated conditions such as cancer. Based on the results of the present study, further trials are warranted to determine if the use of beta glucans might be of use in the treatment of a number of chronic human inflammatory conditions

### Principal Components Analysis (PCA)

Principal components analysis (PCA) is a widely used technique for analyzing metabolomic data. It is a simple non-parametric method that can represent the multidimensional Nuclear magnetic resonance spectroscopy (NMR) or mass spectrometry (MS) spectra into lower dimensional space, thereby providing a reduced dimensional representation of the original data, which can be easily visualized and analyzed (Nyamundanda, G., Brennan, L. & Gormley, I. C. Probabilistic principal component analysis for metabolomic data. BMC Bioinformatics 11, 571 (2010). https://doi.org/10.1186/1471-2105-11-571).

Legend: Principal component analysis (PCA) using the peak heights of all the intervention groups after normalization resulted in the Score plot and Loading plot shown in the FIGS. 30A and B. The contribution rate of the first principal component is 67.9%, and that of the second principal component. The contribution rate was 21.7%. The contribution represents the intervention effect. The first principal component is the direction in space along which projections have the largest variance. The second principal component is the direction which maximizes variance among all directions orthogonal to the first (https://www.stat.cmu.edu/˜cshalizi/uADA/12/lectures/ch18.pdf).

TABLE 6
Score plot (FIG. 30A)

| | | 1st principal | 2nd principal |
|---|---|---|---|
| Primary | | component axis | component axis |
| ID | Class | M2.t [1]] | M2.t [2] |
| 2_1 | F18S-2 | 1930.36 | −115.327 |
| 4_1 | F18S-4 | 963.92 | −1136.28 |
| 6_1 | F18S-6 | 1611.59 | 946.564 |
| 8_1 | F18S-8 | 1842.19 | 311.275 |
| 10_1 | F18S-10 | 787.436 | −863.571 |
| 12_1 | F18S-12 | −2180.65 | −610.406 |
| 14_1 | F18S-14 | −1203.22 | 422.6 |
| 16_1 | F18S-16 | −900.037 | 892.627 |
| 18_1 | F18S-18 | −1681.37 | 2287.4 |
| 20_1 | F18S-20 | −1450.56 | −2024.74 |
| 2_2 | F18S-2 | 1952.52 | −51.1662 |
| 4_2 | F18S-4 | 1142.65 | −937.128 |
| 6_2 | F18S-6 | 1756.76 | 960.431 |
| 8_2 | F18S-8 | 1922.12 | 271.428 |
| 10_2 | F18S-10 | 642.562 | −1017.21 |
| 12_2 | F18S-12 | −2255.42 | −687.695 |
| 14_2 | F18S-14 | −1269.07 | 283.687 |
| 16_2 | F18S-16 | −946.579 | 742.18 |
| 18_2 | F18S-18 | −1440.52 | 2315.86 |
| 20_2 | F18S-20 | −1393.58 | −2052.06 |
| 2_3 | F18S-2 | 1957.91 | −107.533 |
| 4_3 | F18S-4 | 993.467 | −1038.85 |
| 6_3 | F18S-6 | 1699.82 | 1058.17 |
| 8_3 | F18S-8 | 1940.23 | 286.173 |
| 10_3 | F18S-10 | 724.865 | −887.018 |
| 12_3 | F18S-12 | −2169.8 | −708.536 |
| 14_3 | F18S-14 | −1411.3 | 373.849 |
| 16_3 | F18S-16 | −824.773 | 888.649 |
| 18_3 | F18S-18 | −1390.32 | 2250.61 |
| 20_3 | F18S-20 | −1351.22 | −2053.98 |

TABLE 7
Loading plot (FIG. 30B)

| | 1st principal | 2nd principal |
|---|---|---|
| | component axis | component axis |
| Primary ID | M2.p [1] | M2.p[2] |
| 2-Aminobutyric acid-2TMS | −0.05266 | −0.01939 |
| 2-Hydroxyisobutyric acid-2TMS | −0.14398 | 0.04664 |
| 3-Aminoglutaric acid-3TMS | −0.03076 | −0.0197 |
| 3-Hydroxybutyric acid-2TMS | 0.0607 | 0.002452 |
| 3-Methyl-2-oxovaleric acid-meto-TMS | −0.04062 | −0.00093 |
| 4-Hydroxyphenylacetic acid-2TMS | −0.03532 | 0.060276 |
| 5-Aminovaleric acid-3TMS | 0.005393 | 0.116583 |
| 5-Oxoproline-2TMS | −0.11873 | −0.06005 |
| Acetylglycine-TMS | −0.05243 | −0.05029 |
| Alanine-2TMS | −0.25373 | −0.19082 |
| Asparagine-3TMS | 0.008404 | −0.07356 |
| Aspartic acid-3TMS | −0.15904 | −0.12938 |
| Fructose-meto-5TMS | 0.054892 | 0.553348 |
| Fucose-meto-4TMS | 0.03972 | 0.002678 |
| Galactose-meto-5TMS | −0.24292 | 0.337048 |
| Glucose-meto-5TMS | −0.39726 | 0.103791 |
| Glutamic acid-3TMS | −0.23821 | −0.15351 |
| Glutamine-3TMS | −0.04591 | −0.04732 |
| Glycerol-3TMS | −0.17402 | 0.027066 |
| Glycine-3TMS | −0.03957 | −0.1629 |
| Hypoxanthine-2TMS | −0.08577 | 0.001902 |
| Inosine-4TMS | −0.04099 | 0.03431 |
| Isoleucine-2TMS | −0.06266 | −0.168 |
| Lactic acid-2TMS | −0.01387 | 0.0969 |
| Leucine-2TMS | −0.08534 | −0.21694 |
| Lysine-4TMS | −0.27637 | −0.05785 |
| Malic acid-3TMS | −0.07896 | 0.054294 |
| Mannose-meto-5TMS | −0.09335 | 0.10573 |
| Methionine-2TMS | −0.07529 | −0.0911 |
| N-Acetylmannosamine-meto-4TMS | −0.10405 | 0.090473 |
| Nicotinic acid-TMS | −0.05149 | −0.00325 |
| Norvaline-TMS | −0.03602 | −0.06112 |
| Ornithine-4TMS | −0.06541 | 0.041485 |
| Phenylalanine-2TMS | −0.11103 | −0.12946 |
| Phosphoric acid-3TMS | −0.31197 | 0.270456 |
| Proline-2TMS | −0.05275 | −0.1001 |
| Putrescine-4TMS | 0.100887 | 0.017203 |
| Pyruvic acid-meto-TMS | −0.11549 | 0.029063 |
| Ribonic acid-5TMS | 0.041062 | 0.046793 |
| Ribose-meto-4TMS | −0.27045 | −0.08063 |
| Serine-3TMS | −0.04611 | −0.12565 |
| Spermidine-5TMS | 0.00252 | −0.00348 |
| Succinic acid-2TMS | −0.18185 | 0.221622 |
| Taurine-3TMS | −0.07693 | 0.032301 |
| Threonine-3TMS | −0.07246 | −0.12375 |
| Thymine-2TMS | −0.02711 | 0.020664 |
| Tryptophan-3TMS | −0.01537 | −0.03776 |
| Tyrosine-3TMS | −0.15409 | −0.07131 |
| Unknown01 | −0.04444 | −0.10761 |
| Unknown02 | −0.0712 | 0.08819 |
| Unknown03 | −0.03324 | −0.06054 |
| Unknown04 | −0.05094 | −0.04467 |
| Unknown05 | −0.0539 | −0.01027 |
| Unknown06 | −0.04572 | 0.004843 |
| Unknown07 | −0.9516 | −0.01389 |
| Unknown08 | −0.0735 | 0.073928 |
| Unknown09 | −0.01915 | −0.00465 |
| Unknown10 | −0.06159 | 0.015619 |
| Unknown11 | 0.010252 | 0.034549 |
| Unknown12 | −0.00311 | 0.031788 |
| Unknown13 | −0.04434 | 0.021046 |
| Unknown14 | −0.04997 | 0.027864 |
| Unknown15 | 0.002428 | −0.00376 |
| Unknown16 | −0.05647 | 0.019535 |
| Unknown17 | −0.03284 | 0.005477 |
| Unknown18 | −0.04692 | 0.011079 |
| Unknown19 | −0.04507 | 0.012537 |
| Unknown20 | 0.011282 | −0.00159 |
| Unknown21 | −0.02904 | 0.028192 |
| Unknown22 | −0.05868 | 0.041112 |
| Unknown23 | −0.05127 | 0.024218 |
| Unknown24 | −0.00259 | −0.00298 |
| Unknown25 | −0.02952 | −0.03112 |
| Unknown26 | −0.03208 | 0.00788 |
| Unknown27 | −0.02526 | −0.01883 |
| Unknown28 | −0.04911 | 0.019965 |
| Unknown29 | −0.05142 | 0.020068 |
| Unknown30 | −0.05729 | 0.020711 |
| Unknown31 | −0.08737 | 0.029155 |
| Unknown32 | −0.05941 | 0.041755 |
| Unknown33 | −0.02893 | 0.001176 |
| Unknown34 | −0.06022 | −0.02171 |
| Unknown35 | −0.04937 | 0.021441 |
| Unknown36 | −0.03112 | −0.0021 |
| Unknown37 | −0.03592 | 0.024374 |
| Unknown38 | −0.03147 | −0.02035 |
| Unknown39 | −0.02819 | 0.002431 |
| Unknown40 | −0.0418 | −0.02118 |
| Unknown41 | −0.04433 | 0.0067 |
| Unknown42 | −0.04188 | 0.002822 |
| Unknown43 | −0.03006 | 0.019347 |
| Unknown44 | −0.03778 | 0.007629 |
| Unknown45 | −0.04395 | 0.008462 |
| Unknown46 | −0.00395 | 0.088413 |
| Unknown47 | −0.05385 | −0.04528 |
| Unknown48 | −0.02045 | −0.01151 |
| Uracil-2TMS | −0.07823 | −0.01942 |
| Valine-2TMS | −0.08136 | −0.18611 |
| Xanthine-3TMS | −0.07982 | −0.0046 |
| Xylose-meto-4TMS | −0.11059 | −0.12946 |

(Advantages of N-163 Beta Glucan on anti-inflammatory activity of liver, kidney and lung that helps resolve fibrosis)
F18S Study. (Study in STAM animal model of NASH disease)

### Methods

In the STAM™ murine model of NASH, four groups were studied for eight weeks-(1) vehicle (RO water), (2) N-163 beta glucan, (3) AFO-202+N-163 beta glucan, (4) telmisartan (standard pharmacological intervention).

### Methods-RT-PCR

Total RNA was extracted from liver and ileum samples using RNAiso (Takara Bio, Japan) according to the manufacturer's instructions. One μg of RNA was reverse-transcribed using a reaction mixture containing 4.4 mM MgCl₂ (F. Hoffmann-La Roche, Switzerland), 40 U RNase inhibitor (Toyobo, Japan), 0.5 mM dNTP (Promega, USA), 6.28 UM random hexamer (Promega), 5× first strand buffer (Promega), 10 mM dithiothreitol (Invitrogen, USA) and 200 U MMLV-RT (Invitrogen) in a final volume of 20 μL. The reaction was carried out for 1 hour at 37° C., followed by 5 minutes at 99° C. Real-time PCR was performed using real-time PCR DICE and TB Green™ Premix Ex Taq™ II (Takara Bio). To calculate the relative mRNA expression level, the expression of each gene (TNF-α, MCP-1, α-SMA, TIMP-1, PPARα, TGF-β, IL-6) was normalized to that of reference gene 36B4 (gene symbol: Rplp0). Information of PCR-primer sets and the plate layout are described in Table 8.

TABLE 8
Information of PCR primers

| Gene | Set ID | Sequence |
|---|---|---|
| 36B4 | MA057856 | forward 5′-TTCCAGGCTTTGGGCATCA-3′ (SEQ ID NO: 3) |
| | | reverse 5′-ATGTTCAGCATGTTCAGCAGTGTG-3′ (SEQ ID NO: 4) |
| TNF-α | MA117190 | forward 5′-TATGGCCCAGACCCTCACA-3′ (SEQ ID NO: 5) |
| | | reverse 5′-GGAGTAGACAAGGTACAACCCATC-3′ (SEQ ID NO: 6) |
| MCP-1 | MA066003 | forward 5′-GCATCCACGTGTTGGCTCA-3′ (SEQ ID NO: 7) |
| | | reverse 5′-CTCCAGCCTACTCATTGGGATCA-3′ (SEQ ID NO: 8) |
| Alpha-SMA | MA057911 | forward 5′-AAGAGCATCCGACACTGCTGAC-3′ (SEQ ID NO: 9) |
| | | reverse 5′-AGCACAGCCTGAATAGCCACATAC-3′ (SEQ ID NO: 10) |
| TIMP-1 | MA098519 | forward 5′-TGAGCCCTGCTCAGCAAAGA-3′ (SEQ ID NO: 11) |
| | | reverse 5′-GAGGACCTGATCCGTCCACAA-3′ (SEQ ID NO: 12) |
| PPARα | MA089025 | forward 5′-AAGTGCCTGTCTGTCGGGATG-3′ (SEQ ID NO: 13) |
| | | reverse 5′-CCAGAGATTTGAGGTCTGCAGTTTC-3′ (SEQ ID NO: 14) |
| TGF-β | MA030397 | forward 5′-GTGTGGAGCAACATGTGGAACTCTA-3′ (SEQ ID NO: 15) |
| | | reverse 5′-TTGGTTCAGCCACTGCCGTA-3′ (SEQ ID NO: 16) |
| IL-6 | MA152279 | forward 5′-CAACGATGATGCACTTGCAGA-3′ (SEQ ID NO: 17) |
| | | reverse 5′-CTCCAGGTAGCTATGGTACTCCAGA-3′ (SEQ ID NO: 18) |

36B4: Ribosomal protein, large, P0 (Rplp0)
TNF-α: Tumor necrosis factor (Tnf)
MCP-1: Chemokine (C-C motif) ligand 2 (Ccl2)
Alpha-SMA: Actin, alpha 2, smooth muscle, aorta (Acta2)
TMP-1: Tissue inhibitor of metatoproteinase 1 (Timp1)
PPARα: Peroxisome proliferator activated receptor alpha (Ppara)
TGF-β: Transforming growth factor, beta 1 (Tbfb1)
IL-6: Interleukin 6 (Il6)

### Results

Decrease in Free fatty Acids (FFA) and Alpha-SMA both being associated with development of NASH followed by cirrhosis is greater in N-163 beta glucan group.

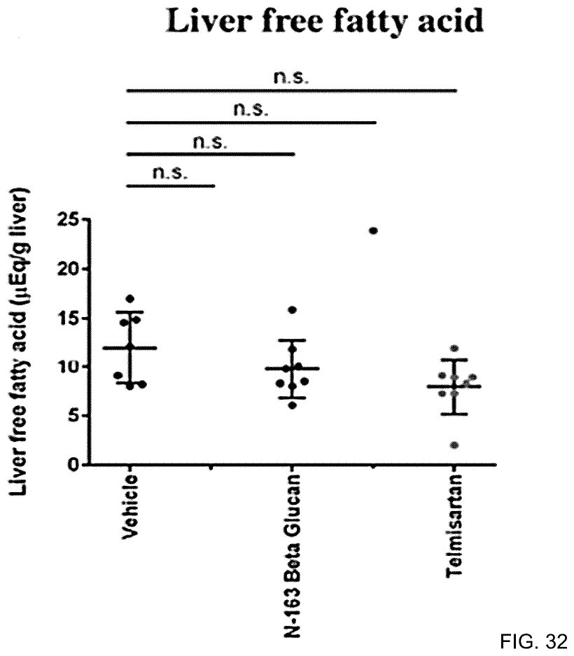

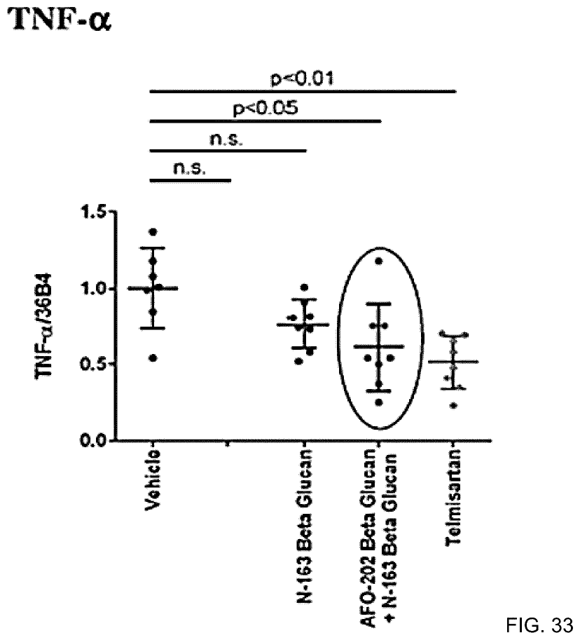

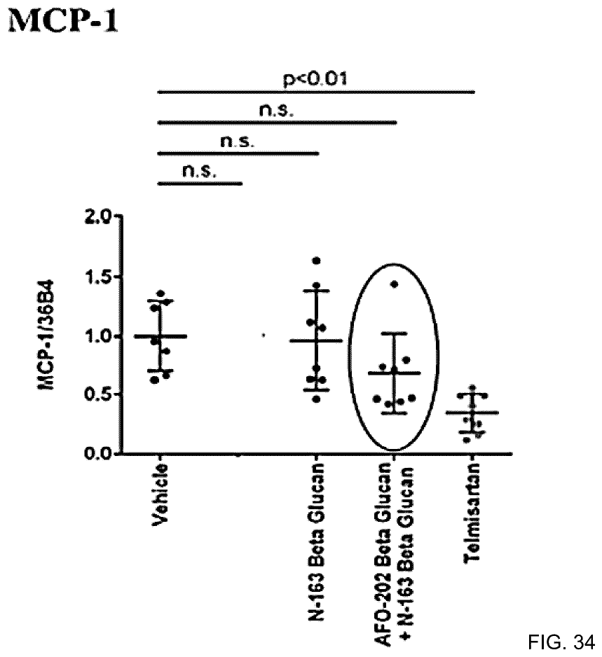

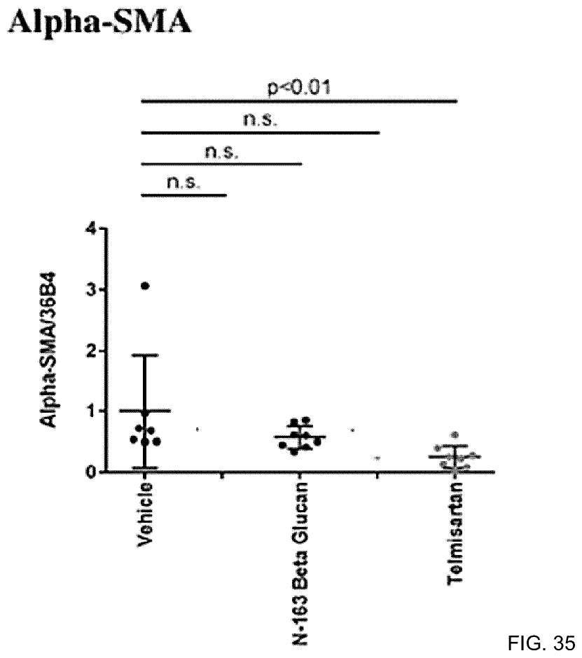

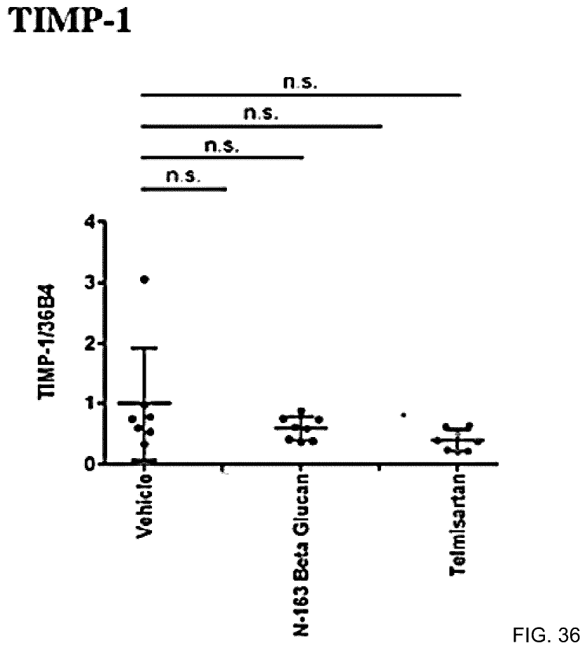

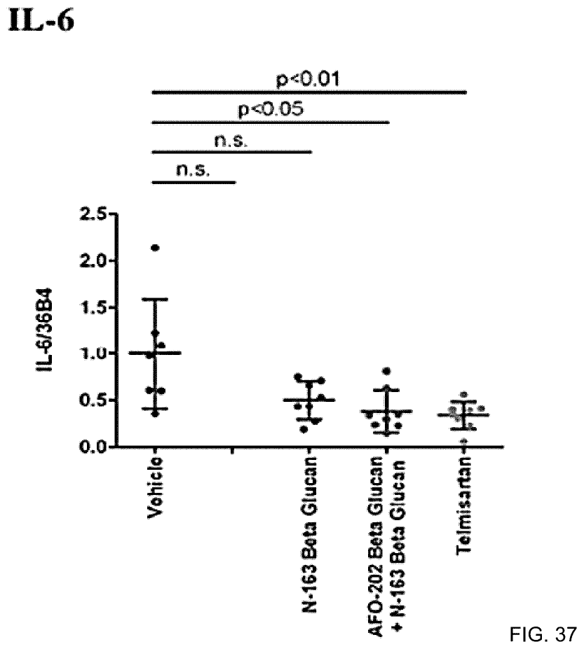

Greater decrease in TNF-Alpha, TIMP-1 and MIP-2 in N-163+AFO-202 beta glucan group than N-163 group makes the combination a highly potential agent against inflammation, liver injury and fibrosis [FIG. 32-37].

### Discussion

When mRNA markers of lipid metabolism and inflammation were studied in the NASH model of mice after administration of N-163 and combination of AFO-202 and N-163, compared with control and Telmisartan. Hepatic mRNA markers of inflammation (TNF-α, MIP-1) in the combination of N-163+AFO-202 beta glucan group makes it an ideal adjuvant for general inflammation and also in prevention of liver injury and fibrosis. mRNA markers of lipogenesis and lipid modulation and fibrosis (TIMP-1) were significant in N-163 group alone compared to the combination of beta glucans.

### Methods—ELISA

Plasma MIP2 levels were measured by commercial ELISA kit. ELISA kits are shown in Table 9.

TABLE 9

| | |
|---|---|
| MIP2 | Name: Mouse MIP2 ELISA Kit (CXCL2) |
| | Manufacture: Abcam |
| | Lot #: GR3412247-6 |
| | Cat #: ab204517 |

### Results

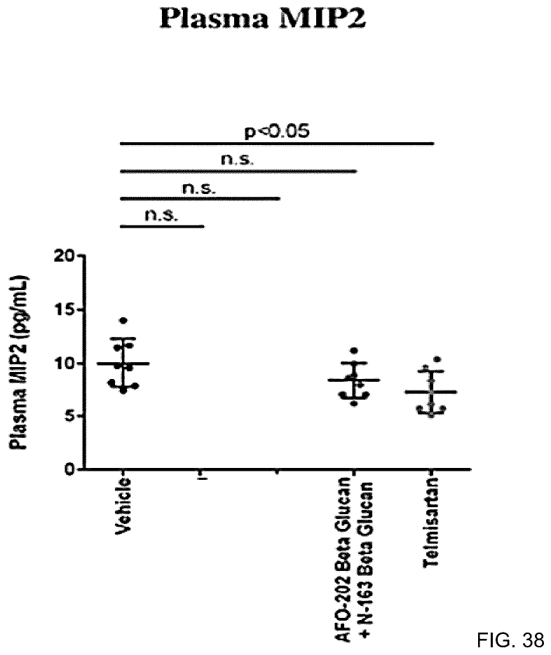

There was greater Decrease in MIP-2 in N-163+AFO-202 beta glucan group than N-163 group [FIG. 38].

### Discussion

Macrophage inflammatory protein (MIP)-2 is produced in response to infection or injury. It plays a key role in the development of liver diseases as higher concentrations mediates liver inflammation (Ref: 10.3748/wjg.v23.i17.3043). Decrease in MIP-2 in combination of N-163+AFO-202 beta glucan group than N-163 group will help to make this n adjunct for treatment of NASH and NAFLD.

(Different Beta Glucans Comparison with N-163-KUWH Data)

The morphology of the normal human dendritic cells (NHDC: CC-2701) expressing HLA-DR, CD11C, CD86, CD80, and CD14 treated with five types of beta-glucans (Control: Phosphate buffered saline (PBS); BG-2: N-163; BG-3: Micelle Glucan® (gel or liquid type) purchased from RL-JP Co., Japan; BG-4: Beta-glucan NEW EX (gel or liquid type) purchased from Aureo BIS, Japan; BG-5: Yeast Glucan (capsule, inside powder?) purchased from Shell Life Japan Co., Japan).

### Results

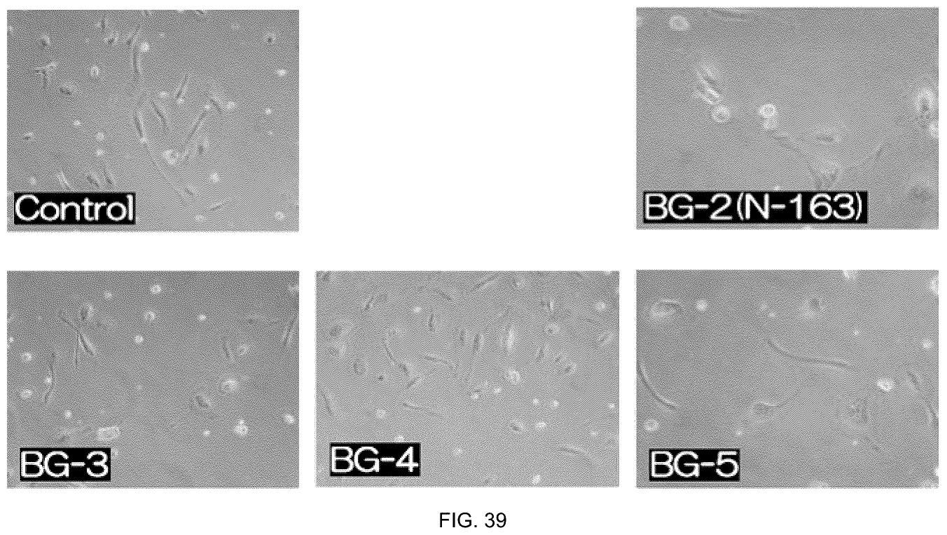

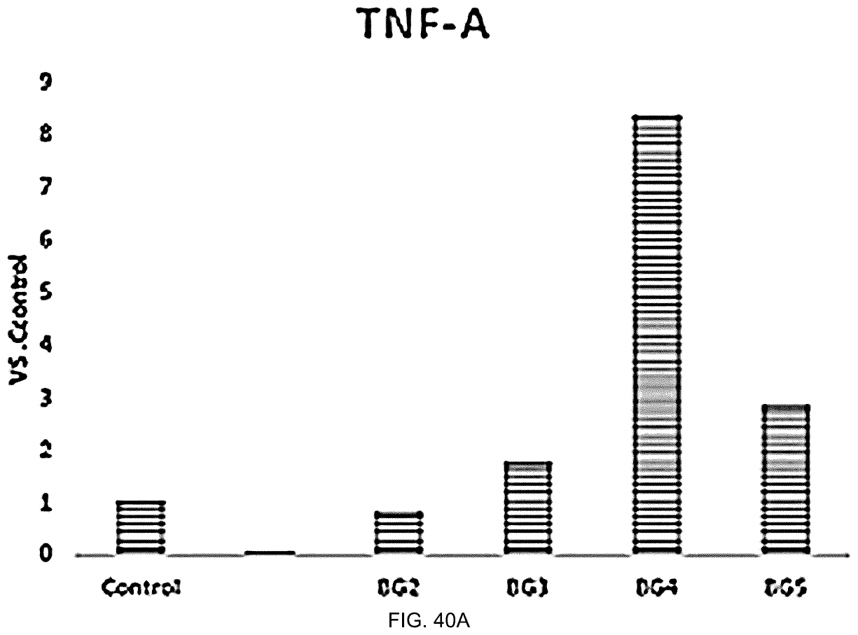

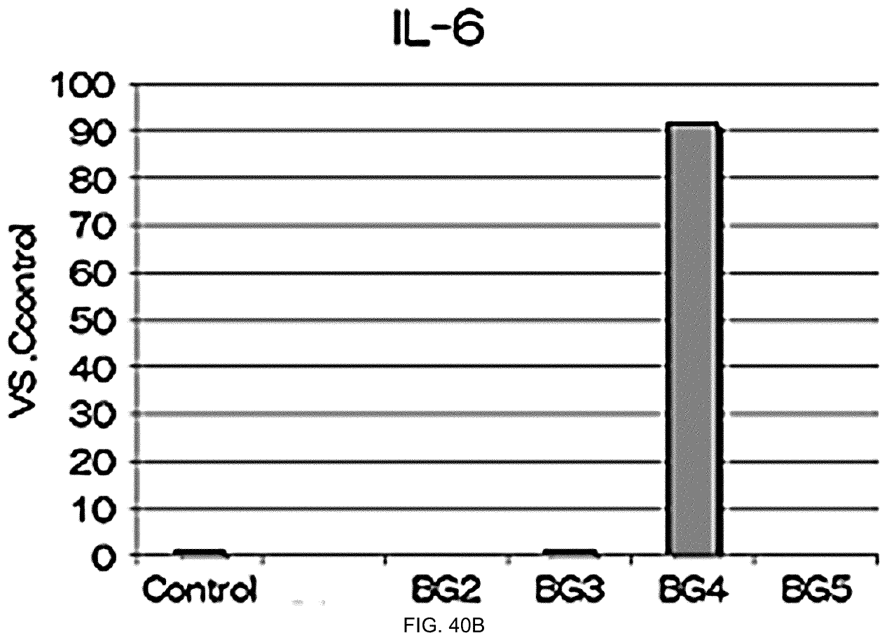

Decrease in IL-4, 10 and 13 were highest in N-163 group [FIGS. 39 and 40].

### Discussion

Interleukin 4, 10 and 13 mediates important pro-inflammatory functions (Ref: doi: 10.1186/rr40; https://www.jimmunol.org/content/165/5/2783;
https://www.frontiersin.org/articles/10.3389/fmed.2017.00139/full). Therefore, decrease in these interleukins after addition of N-163 beta glucan on normal human dendritic cells prove the efficacy of this beta glucan as a potent-anti-inflammatory adjuvant.

mRNA expression of IL-4, IL-10 and IL-13 on the normal human dendritic cells (NHDC: CC-2701) expressing HLA-DR, CD11C, CD86, CD80 and CD14 treated with beta-glucans

The normal human dendritic cells (NHDC CC-2701: LONZA Co.) was treated with several beta-glucans (Control: Phosphate buffered saline (PBS); BG-1: N-163; BG-2: Beta-glucan NEW EX (gel or liquid type) purchased from Aureo BIS, Japan; BG-3: Yeast Glucan (capsule, inside powder?) purchased from Shell Life Japan Co., Japan) (final concentration: 50 μg/mL) for 4 days. The mRNA expression of IL-4 was tested using an RT-PCR method.

### Results

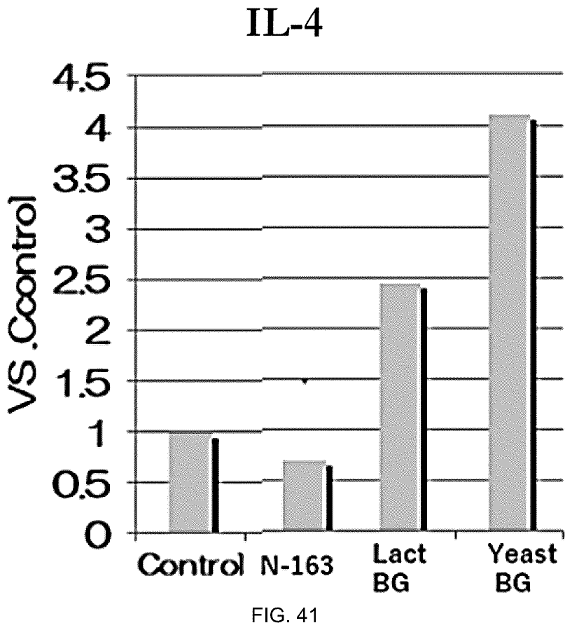

The results are shown in FIG. 41. Decrease in IL-4 significant in N-163 BG

### Discussion

Interleukin 4 mediates important pro-inflammatory functions in asthma, including induction of isotype rearrangement of IgE, expression of VCAM-1 molecules (vascular cell adhesion molecule 1), promoting eosinophilic transmigration through endothelium, mucus secretion and T helper type 2 (Th2) leading to cytokine release [C1]. Therefore, decrease in IL-4 in N-163 is beneficial against inflammation.

The normal human dendritic cells (NHDC CC-2701: LONZA Co.) was treated with several beta-glucans (Control: Phosphate buffered saline (PBS); BG-1: N-163BG; BG-2: Micelle Glucan® (gel or liquid type) purchased from RL-JP Co., Japan; BG-3: Yeast Glucan (capsule, inside powder?) purchased from Shell Life Japan Co., Japan) (final concentration: 50 μg/mL) for 4 days. The mRNA expression of IL-10 was tested using an RT-PCR method.

### Results

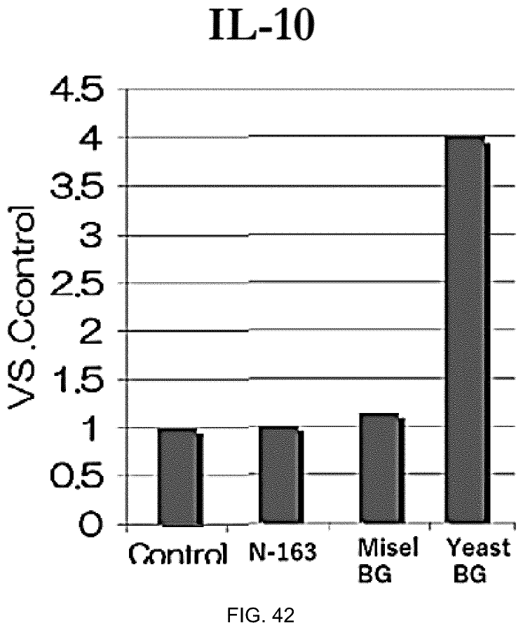

The results are shown in FIG. 42. Decrease in IL-10 greatest in BG1-N-163

### Discussion

### Proinflammatory Effects of IL-10

During Human EndotoxemiaIL-10 is considered a potent anti-inflammatory cytokine that strongly inhibits the production of proinflammatory cytokines. Recent studies have suggested that IL-10 also has immunostimulatory properties on CD4+, CD8+ T cells, and/or NK cells, resulting in increased IFN-γ production. These data indicate that high-dose IL-10 treatment in patients with inflammatory disorders can be associated with undesired proinflammatory effects [C2]. Therefore, decrease in IL-4 in BG1-N-163 is beneficial.

The normal human dendritic cells (NHDC CC-2701: LONZA Co.) was treated with several beta-glucans (Control: Phosphate buffered saline (PBS); BG-1: N-163BG; BG-2: Beta-glucan NEW EX (gel or liquid type) purchased from Aureo BIS, Japan; BG-3: Yeast Glucan (capsule, inside powder?) purchased from Shell Life Japan Co., Japan) (final concentration: 50 μg/mL) for 4 days. The mRNA expression of IL-13 was tested using an RT-PCR method.

### Results

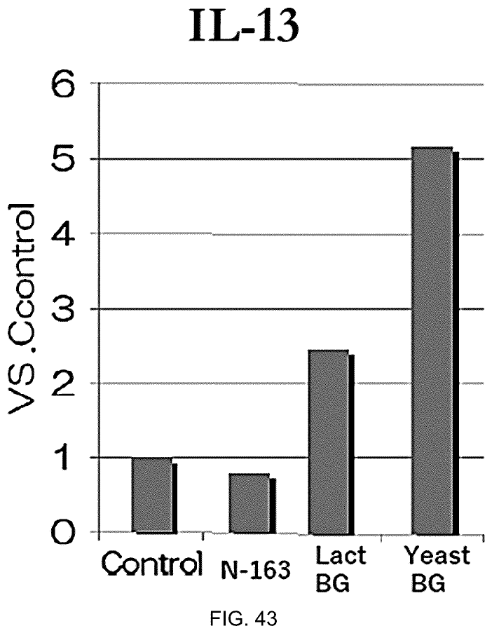

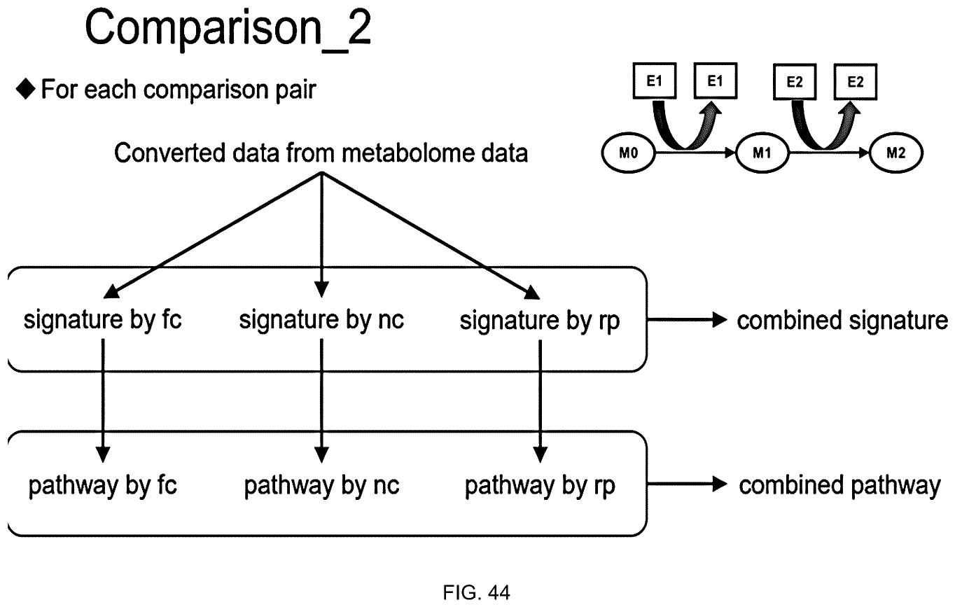

The results are shown in FIG. 43. Decrease in IL-13 significant in N-163 BG

### Discussion

IL-13 is a pleiotropic type 2 cytokine that has been shown to be integral in the pathogenesis of asthma and other eosinophilic disorders. IL-13 levels are elevated in animal models of eosinophilic inflammation and in the blood and tissue of patients diagnosed with eosinophilic disorders[C3]. Therefore, decrease in IL-13 in BG1-N-163 is beneficial.

### (MoA(Mechanism of Action; Pathway/Signature) Evaluation)

This evaluation was based on the results of Metabolome analysis of F18S study.

### Methods

### 1. Title

Elucidation of MoA of β-glucan related compounds [Analysis No.: SC SC22020201] 2.

### 2. Purpose of Analysis

To elucidate the MoAs of β-glucan-related compounds based on metabolomics analysis (Attachment 1) of metabolome data of β-glucan-related compounds provided by GNC Corporation using Socium's technology.

### 3. Analysis Details

Based on the metabolome data of three β-glucan-related compounds owned by GNC, Socium Corporation's proprietary mathematical information technology will be used to analyze the metabolome data, which will be converted into the enzyme abundance before and after the metabolite formation, and the imprinting and significant pathways will be estimated. Furthermore, the imprinting and pathway information will be aggregated by meta-analysis to provide analytical results that contribute to the elucidation of MoA and target molecules.

### 4. Reagents, Instruments, Analysis Software, and Public Databases Analysis Software Maker Model No.

Compound Eyes In-house developed U.S. Pat. No. 6,356,015

### 5. Database Organization URL

The Human Metabolome Database The Human Metabolome Library (HML) https://hmdb.ca/The
Molecular Signatures Database (MSigDB) Broad Institute http://software.broadinstitute.org/gsea/msigdb-

### Molecular Imprint and Significant Pathways

TABLE 10

| | Up- | Down- |
|---|---|---|
| | regulated | regulated |
| Group | genes | genes |
| Control vs. N-163 group (pre) | 47/38 | 11/12 |
| Control vs. N-163 group (post) | 26/36 | 7/10 |

TABLE 11
Upregulated genes

| | |
|---|---|
| ALDH5A1 | aldehyde dehydrogenase 5 family |
| | member A1 |
| BBOX1 | gamma-butyrobetaine hydroxylase 1 |
| P4HA1 | prolyl 4-hydroxylase subunit alpha 1 |
| P4HA2 | prolyl 4-hydroxylase subunit alpha 2 |
| PHYH | phytanoyl-CoA 2-hydroxylase |
| SDHA | succinate dehydrogenase complex |
| | flavoprotein subunit A |
| SDHB | succinate dehydrogenase complex |
| | tron sulfur subunit B |
| SDHC | succinate dehydrogenase complex |
| | subunit C |
| SDHD | succinate dehydrogenase complex |
| | subunit D |

TABLE 12
Downregulated genes

| | |
|---|---|
| CPT1A | carnitine palmitoyltransferase 1A |
| CPT1B | carnitine palmitoyltransferase 1B |
| CPT2 | carnitine palmitoyltransferase 2 |
| LPL | lipoprotein lipase |
| MGLL | monoglyceride lipase |
| PAPSS1 | 3′-phospho anosine 5-phosphosulfate |
| | synthase 1 |
| PLA2G2E | phospholipase A2 group IIE |

### Result

There was upregulation of ALDH5A1, BBOX1, P4HA1, P4HA2, SDHA, SDHB, SDHC and SDHD genes in N-163 group. Genes such as CPT1A, CPT1B, CPT-2, LPL MGLL, PAPSS1 AND PLA2G2E was down regulated in N-163 group.

### Discussion

Carnitine palmitoyltransferase (CPT) present on outer surface of mitochondria and serves as a regulatory site for fatty acid oxidation. Similarly, down-regulation of genes associated with lipogenesis and dyslipidemia which leads to fatty liver (NASH) makes the N-163 beta glucan effective as a prophylactic agent against NASH. P4HA genes play a major role in liver metabolism. So their upregulation will help in preserving liver function during NASH.

### Modifications and Other Embodiments

Various modifications and variations of the described glucan products, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the chemical, biological, medical, environmental, cosmetic or food arts or related fields are intended to be within the scope of the following claims.

### REFERENCES

A1. Nakashima A, Sugimoto R, Suzuki K, Shirakata Y, Hashiguchi T, Yoshida C, Nakano Y. Anti-fibrotic activity of Euglena gracilis and paramylon in a mouse model of non-alcoholic steatohepatitis. Food Sci Nutr. 2018 Nov. 8; 7(1): 139-147
A2. Oniciu D C, Hashiguchi T, Shibazaki Y, Bisgaier C L. Gemcabene downregulates inflammatory, lipid-altering and cell-signaling genes in the STAM™ model of NASH. PloS One. 2018 May 30; 13(5): e0194568.
A3. Oseini A M, Sanyal A J. Therapies in non-alcoholic steatohepatitis (NASH). Liver Int. 2017 January; 37 Suppl 1(Suppl 1):97-103
A4. Alam S, Kabir J, Mustafa G, Gupta U, Hasan S K, Alam A K. Effect of telmisartan on histological activity and fibrosis of non-alcoholic steatohepatitis: A 1-year randomized control trial. Saudi J Gastroenterol. 2016 January-February; 22(1):69-76.
A5. Yano H. Sophy Beta-Glucan is effective in alleviating increased blood sugar levels. Abstract presented at the 55th Conference of the Japanese Society of Nutrition and Dietetics 2008.
A6. Dedeepiya V D, Sivaraman G, Venkatesh A P, Preethy S, Abraham S J. Potential effects of nichi glucan as a food supplement for diabetes mellitus and hyperlipidemia: preliminary findings from the study on three patients from India. Case Rep Med. 2012; 2012:895370.
A7. Ganesh J S, Rao Y Y, Ravikumar R, Jayakrishnan G A, Iwasaki M, Preethy S, Abraham S J. Beneficial effects of black yeast derived 1-3, 1-6 Beta Glucan-Nichi Glucan in a dyslipidemic individual of Indian origin-a case report. J Diet Suppl 2014:11:1-6.
A8. Starley B Q, Calcagno C J, Harrison S A. Nonalcoholic fatty liver disease and hepatocellular carcinoma: a weighty connection. Hepatology. 2010 May; 51(5): 1820-32.
A9. Dharmalingam M, Yamasandhi P G. Nonalcoholic Fatty Liver Disease and Type 2 Diabetes Mellitus. Indian J Endocrinol Metab. 2018 May-June; 22(3):421-428.
10. Gadge P, Gadge R, Paralkar N, Jain P, Tanna V. Effect of telmisartan on blood pressure in patients of type 2 diabetes with or without complications. Perspect Clin Res. 2018 October-December; 9(4): 155-160.
A11. Hamamoto Y, Honjo S, Kawasaki Y, Ikeda H, Mori K, Fujimoto K, Tatsuoka H, Iwasaki Y, Nomura K, Wada Y, Koshiyama H. Relationship between telmisartan dose and glycaemic control in Japanese patients with type 2 diabetes mellitus and hypertension: a retrospective study. Clin Drug Investig. 2012 Sep. 1; 32(9):577-82.
A12. Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol 2007: 51:861-73.
A13. Zhu Y, Yao Y, Gao Y, Hu Y, Shi Z, Ren G. Suppressive Effects of Barley β-Glucans with Different Molecular Weight on 3T3-L1 Adipocyte Differentiation. J Food Sci. 2016 March; 81(3):H786-93.
A14. Ikewaki N, Dedeepiya V D, Iwasaki M, Abraham S J K. Commentary: Beyond “TRIM” Benefits of β-Glucan by Blood Glucose and Lipid Balancing Potentials in Its Defense Against COVID-19. Front Immunol. 2021 Mar. 29; 12:620658.
A15. Lakatos H F, Thatcher T H, Kottmann R M, Garcia™, Phipps R P, Sime P J. The Role of PPARs in Lung Fibrosis. PPAR Res. 2007; 2007:71323.
A16. Zhang X, Chen X, Hong Q, Lin H, Zhu H, Liu Q, Wang J, Xie Y, Shang X, Shi S, Lu Y, Yin Z. TIMP-1 promotes age-related renal fibrosis through upregulating ICAM-1 in human TIMP-1 transgenic mice. J Gerontol A Biol Sci Med Sci. 2006 November; 61(11):1130-43.
A17. Fielding C A, Jones G W, McLoughlin R M, McLeod L, Hammond V J, Uceda J, Williams A S, Lambie M, Foster T L, Liao C T, Rice C M, Greenhill C J, Colmont C S, Hams E, Coles B, Kift-Morgan A, Newton Z, Craig K J, Williams J D, Williams G T, Davies S J, Humphreys I R, O'Donnell V B, Taylor P R, Jenkins B J, Topley N, Jones S A. Interleukin-6 signaling drives fibrosis in unresolved inflammation. Immunity. 2014 Jan. 16; 40(1):40-50.
A18. Jun J I, Lau L F. Resolution of organ fibrosis. J Clin Invest. 2018 Jan. 2; 128(1):97-107.
A19. Liedtke C, Luedde T, Sauerbruch T, Scholten D, Streetz K, Tacke F, Tolba R, Trautwein C, Trebicka J, Weiskirchen R. Experimental liver fibrosis research: update on animal models, legal issues and translational aspects. Fibrogenesis Tissue Repair. 2013 Oct. 1; 6(1): 19.
B1. Valdes A M, Walter J, Segal E, Spector T D. Role of the gut microbiota in nutrition and health. BMJ. 2018 Jun. 13; 361:k2179.
B2. Zierer J, Jackson M A, Kastenmüller G, Mangino M, Long T, Telenti A, Mohney R P, Small K S, Bell J T, Steves C J, Valdes A M, Spector T D, Menni C. The fecal metabolome as a functional readout of the gut microbiome. Nat Genet. 2018 June; 50(6):790-795.
B3. Dedeepiya V, Sivaraman G, Venkatesh A, Preethy S, Abraham S. Potential Effects of Nichi Glucan as a Food Supplement for Diabetes Mellitus and Hyperlipidemia; Preliminary Findings from the Study on Three Patients from India. Case Reports in Medicine 2012 (2012), Article ID 895370
B4. Ganesh J S, Rao Y Y, Ravikumar R, Jayakrishnan A G, Iwasaki M, Preethy S, Abraham S. Beneficial effects of Black yeast derived 1-3, 1-6 beta glucan-Nichi Glucan in a dyslipidemic individual of Indian origin-A case report. J Diet Suppl. 2014; 11(1): 1-6.
B5. Raghavan K, Dedeepiya V D, Ikewaki N, Sonoda T, Iwasaki M, Preethy S, Abraham S J K. Improvement of behavioural pattern and alpha-synuclein levels in autism spectrum disorder after consumption of a beta-glucan food supplement in a randomized, parallel-group pilot clinical study. BMJ Neurology Open (In print)
B6. Raghavan K, Dedeepiya V D, Kandaswamy R, Balamurugan M, Ikewaki N, Sonoda T, Kurosawa G, Iwasaki M, Preethy S, Abraham S J K. Improvement of sleep patterns and serum melatonin levels in children with autism spectrum disorders after consumption of beta-1,3/1,6-glucan in a pilot clinical study. Research Square rs.3.rs-701988/v1; doi: 10.21203/rs.3.rs-701988/v1
B7. Ikewaki N, Kurosawa G, Iwasaki M, Preethy S, Dedeepiya V D, Vaddi S, Senthilkumar R, Levy G A, Abraham S J K. Hepatoprotective effects of Aureobasidium pullulans derived Beta 1,3-1,6 biological response modifier glucans in a STAM-animal model of non-alcoholic steatohepatitis. bioRxiv 2021.07.08.451700; doi: 10.1101/2021.07.08.451700
B8. Pushkala S, Seshayyan S, Theranirajan E, Sudhakar D, Raghavan K, Dedeepiya V D, Ikewaki N, Iwasaki M, Preethy S, Abraham S. Efficient control of IL-6, CRP and Ferritin in Covid-19 patients with two variants of Beta-1,3-1,6 glucans in combination, within 15 days in an open-label prospective clinical trial. medRxiv 2021.12.14.21267778; doi: 10.1101/2021.12.14.21267778
B9. Raghavan K, Dedeepiya V D, Suryaprakash V, Rao K S, Ikewaki N, Sonoda T, Levy G A, Iwasaki M, Senthilkumar R, Preethy S, Abraham S J K. Beneficial Effects of novel Aureobasidium pullulans strains produced beta-1,3-1,6 glucans on interleukin-6 and D-Dimer levels in COVID-19 patients; results of a randomized multiple-arm pilot clinical study. Biomedicine and Pharmacotherapy 2021. sciencedirect. https://doi.org/10.1016/j.biopha.2021.112243
B10. Raghavan K, Dedeepiya V D, Yamamoto N, Ikewaki N, Sonoda T, Kurosawa G, Iwasaki M, Kandaswamy R, Senthilkumar R, Preethy S, Abraham S J K. Beneficial reconstitution of gut microbiota and control of alpha-synuclein and curli-amyloids-producing enterobacteria, by beta 1,3-1,6 glucans in a clinical pilot study of autism and potentials in neurodegenerative diseases. medRxiv 2021.10.26.21265505; doi: 10.1101/2021.10.26.21265505
B11. Bashir K M I, Choi J S. Clinical and Physiological Perspectives of β-Glucans: The Past, Present, and Future. Int J Mol Sci. 2017 Sep. 5; 18(9): 1906.
B12. Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol. 2007; 51(9):861-73.
B13. Ikewaki N, Onaka T, Ikeue Y, Nagataki M, Kurosawa G, Dedeepiya V D, Rajmohan M, Vaddi S, Senthilkumar R, Preethy S, Abraham S J K. Beneficial effects of the AFO-202 and N-163 strains of Aureobasidium pullulans produced 1,3-1,6 beta glucans on non-esterified fatty acid levels in obese diabetic KKAy mice: A comparative study.bioRxiv 2021.07.22.453362; doi: 10.1101/2021.07.22.453362
B14. Ikewaki N, Sonoda T, Kurosawa G, Iwasaki M, Dedeepiya V D, Senthilkumar R, Preethy S, Abraham S J K. Immune and metabolic beneficial effects of Beta 1,3-1,6 glucans produced by two novel strains of Aureobasidium pullulans in healthy middle-aged Japanese men: An exploratory study. medRxiv 2021.08.05.21261640; doi: 10.1101/2021.08.05.21261640
B15. STAM Model. https://www.smccro-lab.com/service/service_disease_area/stam.html
B16. Nakashima A, Sugimoto R, Suzuki K, Shirakata Y, Hashiguchi T, Yoshida C, Nakano Y. Anti-fibrotic activity of Euglena gracilis and paramylon in a mouse model of non-alcoholic steatohepatitis. Food Sci Nutr. 2018; 7(1): 139-147
B17. Dantzer R. Neuroimmune Interactions: From the Brain to the Immune System and Vice Versa. Physiol Rev. 2018 Jan. 1; 98(1):477-504.
B18. Kolodziejczyk A A, Zheng D, Shibolet O, Elinav E. The role of the microbiome in NAFLD and NASH. EMBO Mol Med. 2019 February; 11(2):e9302. doi: 10.15252/emmm.201809302. PMID: 30591521; PMCID: PMC6365925.
B19. Chen J, Vitetta L. Gut Microbiota Metabolites in NAFLD Pathogenesis and Therapeutic Implications. Int J Mol Sci. 2020 Jul. 23; 21(15):5214. doi: 10.3390/ijms21155214. PMID: 32717871; PMCID: PMC7432372.
B20. Kang D W, Adams J B, Vargason T, Santiago M, Hahn J, Krajmalnik-Brown R. Distinct Fecal and Plasma Metabolites in Children with Autism Spectrum Disorders and Their Modulation after Microbiota Transfer Therapy. mSphere. 2020 Oct. 21; 5(5): e00314-20. doi: 10.1128/mSphere.00314-20. PMID: 33087514; PMCID: PMC7580952.
B21. Vascellari S, Palmas V, Melis M, Pisanu S, Cusano R, Uva P, Perra D, Madau V, Sarchioto M, Oppo V, Simola N, Morelli M, Santoru M L, Atzori L, Melis M, Cossu G, Manzin A. Gut Microbiota and Metabolome Alterations Associated with Parkinson's Disease. mSystems. 2020 Sep. 15; 5(5): e00561-20. doi: 10.1128/mSystems.00561-20. PMID: 32934117; PMCID: PMC7498685.
B22. Bernstein C N, Forbes J D. Gut Microbiome in Inflammatory Bowel Disease and Other Chronic Immune-Mediated Inflammatory Diseases. Inflamm Intest Dis. 2017 November; 2(2): 116-123. doi: 10.1159/000481401. Epub 2017 Oct. 20. PMID: 30018962; PMCID: PMC5988152.B23. Walker A, Schmitt-Kopplin P. The role of fecal sulfur metabolome in inflammatory bowel diseases. Int J Med Microbiol. 2021 July; 311(5): 151513.
B24. Cirstea M, Radisavljevic N, Finlay B B. Good Bug, Bad Bug: Breaking through Microbial Stereotypes. Cell Host Microbe. 2018 Jan. 10; 23(1): 10-13.
B25. Heintz-Buschart A, Pandey U, Wicke T, Sixel-Döring F, Janzen A, Sittig-Wiegand E, Trenkwalder C, Oertel W H, Mollenhauer B, Wilmes P. The nasal and gut microbiome in Parkinson's disease and idiopathic rapid eye movement sleep behavior disorder. Mov Disord. 2018 January; 33(1):88-98. doi: 10.1002/mds.27105. Epub 2017 Aug. 26. PMID: 28843021; PMCID: PMC5811909.
B26. Davis C D. The Gut Microbiome and Its Role in Obesity. Nutr Today. 2016 July-August; 51(4): 167-174. doi: 10.1097/NT.0000000000000167. PMID: 27795585; PMCID: PMC5082693.
B27. Jumpertz R, Le D S, Turnbaugh P J, et al. Energy-balance studies reveal associations between gut microbes, caloric load, and nutrient absorption in humans. Am. J. Clin. Nutr. 2011; 94:58-65.
B28. Yoshimoto S, Mitsuyama E, Yoshida K, Odamaki T, Xiao J Z. Enriched metabolites that potentially promote age-associated diseases in subjects with an elderly-type gut microbiota. Gut Microbes. 2021 January-December; 13(1): 1-11.
B29. Le Gall G, Guttula K, Kellingray L, Tett A J, Ten Hoopen R, Kemsley E K, Savva G M, Ibrahim A, Narbad A. Metabolite quantification of faecal extracts from colorectal cancer patients and healthy controls. Oncotarget. 2018 Sep. 7; 9(70):33278-33289. doi: 10.18632/oncotarget.26022. Erratum in: Oncotarget. 2019 Feb. 26; 10(17): 1660. PMID: 30279959; PMCID: PMC6161785.
B30. Lu K, Dong S, Wu X, Jin R, Chen H. Probiotics in Cancer. Front Oncol. 2021 Mar. 12; 11:638148.
B31. Gibson C M, Childs-Kean L M, Naziruddin Z, Howell C K. The alteration of the gut microbiome by immunosuppressive agents used in solid organ transplantation. Transpl Infect Dis. 2021 February; 23(1):e13397.
C1. John W Steinke, Larry Borish M D, Th2 cytokines and asthma-Interleukin-4: its role in the pathogenesis of asthma, and targeting it for asthma treatment with interleukin-4 receptor antagonists. Respiratory Research 2, Article number: 66 (2001).
C2. Fanny N. Lauw, Dasja Pajkrt, C. Erik Hack, Masashi Kurimoto, Sander J. H. van Deventer and Tom van der Poll. Proinflammatory Effects of IL-10 During Human Endotoxemia. J Immunol Sep. 1, 2000, 165 (5) 2783-2789.
C3. Emma Doran, Fang Cai, Cecile T. J. Holweg, Kit Wong, Jochen Brumm and Joseph R. Arron. Interleukin-13 in Asthma and Other Eosinophilic Disorders. Front. Med., 19 Sep. 2017.

1. A composition for preventing and/or treating fibrosis, comprising a beta-glucan.

2. The composition of claim 1, in which the beta-glucan comprises a beta-glucan produced by Aureobasidium pullulans N-163 (NITE BP-03377).

3. The composition of claim 2, in which the beta-glucan further comprises a beta-glucan produced by Aureobasidium pullulans AFO-202 (FERM BP-19327).

4. The composition of claim 1, in which the beta-glucan consists of a beta-glucan produced by Aureobasidium pullulans N-163 (NITE BP-03377) and a beta-glucan produced by Aureobasidium pullulans AFO-202 (FERM BP-19327).

5. The composition of any one of claim 1, which is used to prevent and/or treat non-alcoholic steatohepatitis (NASH).

6. A composition for improving gut microbiota, comprising a beta-glucan produced by Aureobasidium pullulans N-163 (NITE BP-03377).

7. The composition according to claim 6, further comprising a beta-glucan produced by Aureobasidium pullulans AFO-202 (FERM BP-19327).

8. The composition according to claim 6, wherein the improvement of gut microbiota comprises a decrease of Akkermansia with an increase of beneficial bacteria including Lactobacillus in a gut.

9. The composition according to claim 6, wherein the composition is for prophylactic, ameliorating and/or curative treatment of cancers, and/or fibrosis.

10. A composition for balancing amino acids to beneficial levels, comprising a beta-glucan produced by Aureobasidium pullulans N-163 (NITE BP-03377).

11. The composition according to claim 10, further comprising a beta-glucan produced by Aureobasidium pullulans AFO-202 (FERM BP-19327).

12. The composition according to claim 10, wherein the composition increases Tryptophan and/or decreases Isoleucine, Leucine, and/or Spermidine.

13. The composition according to claim 11, wherein the composition increases Ornithine and/or decreases Tryptophan and/or Phenylalanine.


SEQUENCE LISTING (18 sequences)

SEQ ID NO: 1 - DNA, 34 bp, Artificial Sequence
  Illumina adapter Read 1 3′ end side
ctgtcttctatacacatctccgagcccacgagac

SEQ ID NO: 2 - DNA, 33 bp, Artificial Sequence
  Illumina adapter Read 2 3′ end side
ctgtcttctatacacatctgacgctgccgacga

SEQ ID NO: 3 - DNA, 19 bp, Artificial Sequence
  36B4 forward primer
ttccaggctttgggcatca

SEQ ID NO: 4 - DNA, 24 bp, Artificial Sequence
  36B4 reverse primer
atgttcagcatgttcagcagtgtg

SEQ ID NO: 5 - DNA, 19 bp, Artificial Sequence
  TNF-alpha forward primer
tatggcccagaccctcaca

SEQ ID NO: 6 - DNA, 24 bp, Artificial Sequence
  TNF-alpha reverse primer
ggagtagacaaggtacaacccatc

SEQ ID NO: 7 - DNA, 19 bp, Artificial Sequence
  MCP-1 forward primer
gcatccacgtgttggctca

SEQ ID NO: 8 - DNA, 23 bp, Artificial Sequence
  MCP-1 reverse primer
ctccagcctactcattgggatca

SEQ ID NO: 9 - DNA, 22 bp, Artificial Sequence
  Alpha-SMA forward primer
aagagcatccgacactgctgac

SEQ ID NO: 10 - DNA, 24 bp, Artificial Sequence
  Alpha-SMA reverse primer
agcacagcctgaatagccacatac

SEQ ID NO: 11 - DNA, 20 bp, Artificial Sequence
  TIMP-1 forward primer
tgagccctgctcagcaaaga

SEQ ID NO: 12 - DNA, 21 bp, Artificial Sequence
  TIMP-1 reverse primer
gaggacctgatccgtccacaa

SEQ ID NO: 13 - DNA, 21 bp, Artificial Sequence
  PPAR alpha forward primer
aagtgcctgtctgtcgggatg

SEQ ID NO: 14 - DNA, 25 bp, Artificial Sequence
  PPAR alpha reverse primer
ccagagatttgaggtctgcagtttc

SEQ ID NO: 15 - DNA, 25 bp, Artificial Sequence
  TGF-beta forward primer
gtgtggagcaacatgtggaactcta

SEQ ID NO: 16 - DNA, 20 bp, Artificial Sequence
  TGF-beta reverse primer
ttggttcagccactgccgta

SEQ ID NO: 17 - DNA, 21 bp, Artificial Sequence
  IL-6 forward primer
caacgatgatgcacttgcaga

SEQ ID NO: 18 - DNA, 25 bp, Artificial Sequence
  IL-6 reverse primer
ctccaggtagctatggtactccaga